SEQUENCE LISTING (1 sequence)

SEQ ID NO: 1 - protein, 5 aa, Artificial Sequence
  Synthetic polypeptide
LKKGA

(19) United States
(12) Patent Application Publication
(10) Pub. No.: US 2025/0188065 A1
(43) Pub. Date: Jun. 12, 2025

(54) LIGHT-CONTROLLED BIOORTHOGONAL TETRAZINE LIGATION

(71) Applicant: The Regents of the University Of California, Okland, CA (US)

(72) Inventors: Neal Devaraj, La Jolla, CA (US); Luping Liu, La Jolla, CA (US)

(21) Appl. No.: 18/026,330

(22) PCT Filed: Sep. 28, 2021
(86) PCT No.: PCT/US2021/052458

§ 371 Date: Mar. 14, 2023

Related U.S. Application Data
(60) Provisional application No. 63/085,709, filed on Sep. 30, 2020.

(51) Int. Cl. C07D 405/12; A61K 31/495; A61K 41/00; C07D 405/14; C07F 5/02

(52) U.S. Cl. CPC C07D 405/12; A61K 31/495; A61K 41/0042; C07D 405/14; C07F 5/027

(57) ABSTRACT

Disclosed herein, inter alia, is a light-controlled bioorthogonal tetrazine ligation.

### CROSS-REFERENCES TO RELATED APPLICATIONS

This application claims the benefit of U.S. Provisional Application No. 63/085,709, filed Sep. 30, 2020, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. DP2DK111801 awarded by the National Institutes of Health. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048537-636001WO_Sequence_Listing_ST25.TXT, created Sep. 7, 2021, 480 bytes, machine format IBM-PC, MS Windows operating system, is hereby incorporated by reference.

### BACKGROUND

Bioorthogonal inverse electron demand Diels-Alder reactions between tetrazines and strained dienophiles have found widespread use in applications that include in vivo imaging, drug delivery, metabolic labeling, and polymer modification. The ability to control chemistry using light as a stimulus is perhaps unparalleled with respect to its noninvasive nature and the degree to which spatial and temporal control can be achieved. Light-controlled ligation chemistry could be used to specifically pattern ligands onto biological targets, enable controlled manipulation of biomolecule function, or specifically trigger the release and delivery of bioactive reagents such as therapeutics. Despite this potential, methods to achieve light control over tetrazine ligations has been limited, particularly in the presence of live cells. Disclosed herein, inter alia, are solutions to these and other problems in the art.

### BRIEF SUMMARY

In an aspect is provided a compound (e.g., a photocaged dihydrotetrazine compound), or a pharmaceutically acceptable salt thereof, having the formula:

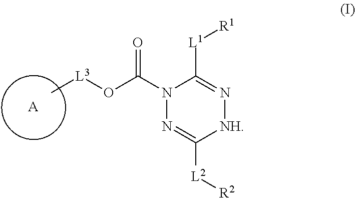

Ring A is a photolabel moiety. L¹ is a bond or covalent linker. L² is a bond or covalent linker. L³ is a bond, substituted or unsubstituted alkylene, or substituted or unsubstituted heteroalkylene.

R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety.

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety.

R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl.

X¹ and X² are independently —F, —Cl, —Br, or —I.

The symbols n1 and n2 are independently an integer from 0 to 4. The symbols m1, m2, v1, and v2 are independently 1 or 2.

In an aspect is provided a pharmaceutical composition including a compound described herein, or a pharmaceutically acceptable salt thereof, and a pharmaceutically acceptable excipient.

In an aspect is provided a method of making a tetrazine compound, said method comprising irradiating a photocaged dihydrotetrazine compound with light.

In an aspect is provided a method of treating a cancer in a subject in need thereof, the method including: (i) administering to the subject in need thereof a photocaged dihydrotetrazine compound or a compound described herein, or a pharmaceutically acceptable salt thereof; (ii) irradiating the photocaged dihydrotetrazine compound or the compound with light to form a tetrazine compound; and (iii) reacting the tetrazine compound with a dienophile covalently linked to a monovalent form of an anti-cancer agent, thereby releasing the anti-cancer agent.

In an aspect is provided a method of delivering a drug or a probe in a subject in need thereof, the method including: (i) administering to the subject in need thereof a photocaged dihydrotetrazine compound or a compound described herein, or a pharmaceutically acceptable salt thereof; (ii) irradiating the photocaged dihydrotetrazine compound or the compound with light to form a tetrazine compound; and (iii) reacting the tetrazine compound with a dienophile covalently linked to a monovalent form of the drug or a monovalent form of the probe, thereby releasing the drug or the probe.

### BRIEF DESCRIPTION OF THE DRAWINGS

FIGS. 1A-1B. Light controlled bioorthogonal tetrazine ligation in living cells. FIG. 1A: Light-triggered formation of tetrazine which reacted rapidly with dienophile trans-cyclooctene (TCO) bearing prodrugs or probes (polygon) in cell culture under ambient conditions to enable prodrug therapy, or spatial-temporal imaging in live cells. FIG. 1B: Synthetic route to photocaged dihydrotetrazine 1a.

FIGS. 2A-2B illustrate in situ formation of tetrazine in aqueous solution. FIG. 2A: Light-triggered formation of tetrazine 2a from photocaged dihydrotetrazine 1a. The reaction was carried out with the irradiation of photocaged dihydrotetrazine 1a (16 μM) by a LED light (405 nm) in PBS (containing 0.1% DMSO) with open air at 37° C. for 2 minutes. FIG. 2B: HPLC spectra of samples taken from the reaction mixture at different time points. Before irradiation by LED light (405 nm) (bottom spectrum) and after irradiation by LED light (405 nm) for 2 minutes (top spectrum).

FIGS. 3A-3C. FIG. 3A: Synthesis of tetrazine-containing peptide. Peptide sequence: LKKGA (SEQ ID NO:1). FIG. 3B: HPLC-MS spectrometry of photocaged-dihydrotetrazine-peptide 1c. Top graph: HPLC spectrum of 1c taken before irradiation. Bottom graph: Mass spectrum of 1c. Expected mass 973.4751 Da, found mass 973.4749 Da. FIG. 3C: HPLC-MS spectrometry of tetrazine-peptide 2c, which was obtained from photocaged-dihydrotetrazine-peptide 1c (10 μM) by a LED light (405 nm) in PBS (containing 0.2% DMSO and 0.2% DMF) with open air at 37° C. for 2 minutes. Top graph: HPLC spectrum of 2c taken after irradiation. Bottom graph: Mass spectrum of 2c. Expected mass 778.4220 Da, found mass 778.4234 Da.

FIGS. 4A-4G. Single-cell remodeling of HeLa S3 cell membranes by photoactivation of tetrazine ligation. FIG. 4A: Cartoon depicting live-cell photoactivation of tetrazine ligation on cellular membranes using photocaged dihydrotetrazine-diacylphospholipid 1d and a trans-cyclooctene modified dye (TCO-Dye). FIG. 4B: Photocaged dihydrotetrazine-diacylphospholipid 1d. FIG. 4C: Reaction scheme of compound 1d with TCO-AF488 to form compound 4a. FIG. 4D: Fluorescence live-cell labeling demonstrating single-cell photoactivation of tetrazine ligation on the cell membrane of a selected HeLa S3 cell. TCO-AF488 was used for tetrazine ligation. Fluorescence channel (AF488) shown on the left, and merged fluorescence and brightfield channels on the right. The area irradiated by the 405 nm laser is denoted by the white dashed circle. FIG. 4E: Reaction scheme of compound 1d with TCO-AF568 to form compound 4b. FIG. 4F: Spatiotemporal photoactivation of tetrazine ligation using TCO-AF568. Fluorescence channel (AF568) shown on the left, and merged fluorescence and brightfield channels on the right. The area irradiated by the 405 nm laser is denoted by the white dashed circle in the merged channel. FIG. 4G: Activation of four groups of cells at different locations inside a 0.75 mm by 0.75 mm square area. TCO-AF568 was used for the tetrazine ligation. Images taken from the merged fluorescence (AF568) and brightfield channels. The areas irradiated by the 405 nm laser are denoted by the white dashed circles. Scale bar: 50 μm.

FIGS. 5A-5B. Light-activated tetrazine prodrug therapy in Hep 3B cancer cells. FIG. 5A: Application of light-controlled tetrazine ligation to release the chemotherapeutic doxorubicin in the presence of living Hep 3B cancer cells. Dox, doxorubicin. FIG. 5B: Cell viability of Hep 3B cancer cells after treatments with photocaged dihydrotetrazine 1a (8 μM), TCO-Dox 3c (5.5 μM), side product 4c (5.5 μM), and Dox 5a (5.5 μM) with or without irradiation by LED light (405 nm, 18 W) for 2 minutes, followed by incubation at 37° C. for 24 hours. Error bars indicate standard error of mean (SEM), which are measured from 3 replicates. Statistically significant differences in cell viability between no treatment and other means are indicated: **P<0.01. ns, not significant.

FIG. 6. Examples of photocaged dihydrotetrazines. The functional alkyne group of photo-dihydrotetrazine 1a can be used to label probes such as amino acids (1b), peptides (1c), lipids (1d), proteins, or DNA.

FIG. 7. Examples of photocaged dihydrotetrazines: blue-light-cleavable ortho-nitrophenyl methyl photoprotecting dihydrotetrazine, 6-nitropiperonyl methyl photoprotecting dihydrotetrazine, diethylaminocoumarin photoprotecting dihydrotetrazine, green-light-cleavable BODIPY photoprotecting dihydrotetrazine.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

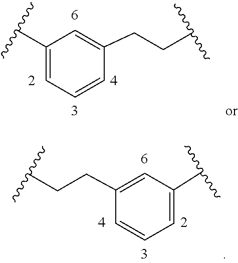 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, =O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₅ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁-C₈alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R¹⁰⁰. As a further example, RA may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1}, R^{L100.1}) may be further substituted with one or more second substituent groups (e.g., R^{1.2}, R^{2.2}, R^{3.2}, R^{4.2}, R^{5.2} . . . R^{100.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{1000.2}; R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

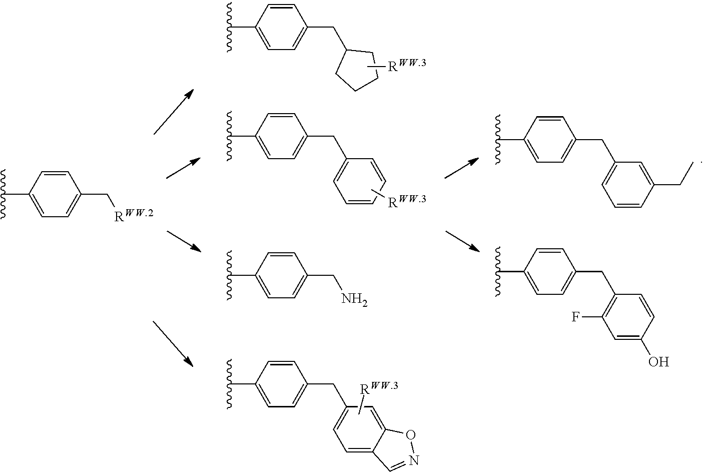

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered), R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2}, and R^{WW.3} is R^{100B.3}. R^{WW.1}, R^{WW.2} and R^{WW.3} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{L}WW 0.3-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

The term “bioconjugate reactive moiety” or “bioconjugate reactive group” refers to a chemical moiety which participates in a reaction to form bioconjugate linker (e.g., covalent linker) or the resulting association between atoms or molecules of bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound. A “derivative” is a compound derived from a chemical compound via a chemical reaction. A derivative of a compound described herein may refer to the compound described herein with the addition or removal of a substituent.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently. When a substituent or linker (e.g., an R group or an L linker) appears multiple times, each appearance of that substituent or linker can be different, i.e., each occurrence of the substituent or the linker may be independently a member of the Markush group for that variable, wherein each occurrence may be optionally different.

A “detectable agent” or “detectable moiety” or “probe” is an atom, molecule, substance, or composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition. A probe moiety is a monovalent probe or a probe capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Examples of detectable agents include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent moiety or fluorescent dye moiety. In embodiments, the detectable moiety is a fluorescein isothiocyanate moiety, tetramethylrhodamine-5-(and 6)-isothiocyanate moiety, Cy2 moiety, Cy3 moiety, Cy5 moiety, Cy7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable moiety is a Indo-1, Ca saturated moiety, Indo-1 Ca2+ moiety, Cascade Blue BSA pH 7.0 moiety, Cascade Blue moiety, LysoTracker Blue moiety, Alexa 405 moiety, LysoSensor Blue pH 5.0 moiety, LysoSensor Blue moiety, DyLight 405 moiety, DyLight 350 moiety, BFP (Blue Fluorescent Protein) moiety, Alexa 350 moiety, 7-Amino-4-methylcoumarin pH 7.0 moiety, Amino Coumarin moiety, AMCA conjugate moiety, Coumarin moiety, 7-Hydroxy-4-methylcoumarin moiety, 7-Hydroxy-4-methylcoumarin pH 9.0 moiety, 6,8-Difluoro-7-hydroxy-4-methylcoumarin pH 9.0 moiety, Hoechst 33342 moiety, Pacific Blue moiety, Hoechst 33258 moiety, Hoechst 33258-DNA moiety, Pacific Blue antibody conjugate pH 8.0 moiety, PO-PRO-1 moiety, PO-PRO-1-DNA moiety, POPO-1 moiety, POPO-1-DNA moiety, DAPI-DNA moiety, DAPI moiety, Marina Blue moiety, SYTOX Blue-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO-1-DNA moiety, BOPRO-1 moiety, BOBO-1-DNA moiety, SYTO 45-DNA moiety, evoglow-Pp1 moiety, evoglow-Bs1 moiety, evoglow-Bs2 moiety, Auramine O moiety, DiO moiety, LysoSensor Green pH 5.0 moiety, Cy 2 moiety, LysoSensor Green moiety, Fura-2, high Ca moiety, Fura-2 Ca2+sup> moiety, SYTO 13-DNA moiety, YO-PRO-1-DNA moiety, YOYO-1-DNA moiety, eGFP (Enhanced Green Fluorescent Protein) moiety, LysoTracker Green moiety, GFP (S65T) moiety, BODIPY FL, MeOH moiety, Sapphire moiety, BODIPY FL conjugate moiety, MitoTracker Green moiety, MitoTracker Green FM, MeOH moiety, Fluorescein 0.1 M NaOH moiety, Calcein pH 9.0 moiety, Fluorescein pH 9.0 moiety, Calcein moiety, Fura-2, no Ca moiety, Fluo-4 moiety, FDA moiety, DTAF moiety, Fluorescein moiety, CFDA moiety, FITC moiety, Alexa Fluor 488 hydrazide-water moiety, DyLight 488 moiety, 5-FAM pH 9.0 moiety, Alexa 488 moiety, Rhodamine 110 moiety, Rhodamine 110 pH 7.0 moiety, Acridine Orange moiety, BCECF pH 5.5 moiety, PicoGreendsDNA quantitation reagent moiety, SYBR Green I moiety, Rhodaminen Green pH 7.0 moiety, CyQUANT GR-DNA moiety, NeuroTrace 500/525, green fluorescent Nissl stain-RNA moiety, DansylCadaverine moiety, Fluoro-Emerald moiety, Nissl moiety, Fluorescein dextran pH 8.0 moiety, Rhodamine Green moiety, 5-(and-6)-Carboxy-2′,7′-dichlorofluorescein pH 9.0 moiety, DansylCadaverine, MeOH moiety, eYFP (Enhanced Yellow Fluorescent Protein) moiety, Oregon Green 488 moiety, Fluo-3 moiety, BCECF pH 9.0 moiety, SBFI-Na+ moiety, Fluo-3 Ca2+ moiety, Rhodamine 123 MeOH moiety, FlAsH moiety, Calcium Green-1 Ca2+ moiety, Magnesium Green moiety, DM-NERF pH 4.0 moiety, Calcium Green moiety, Citrine moiety, LysoSensor Yellow pH 9.0 moiety, TO-PRO-1-DNA moiety, Magnesium Green Mg2+ moiety, Sodium Green Na+ moiety, TOTO-1-DNA moiety, Oregon Green 514 moiety, Oregon Green 514 antibody conjugate pH 8.0 moiety, NBD-X moiety, DM-NERF pH 7.0 moiety, NBD-X, MeOH moiety, CI-NERF pH 6.0 moiety, Alexa 430 moiety, CI-NERF pH 2.5 moiety, Lucifer Yellow, CH moiety, LysoSensor Yellow pH 3.0 moiety, 6-TET, SE pH 9.0 moiety, Eosin antibody conjugate pH 8.0 moiety, Eosin moiety, 6-Carboxyrhodamine 6G pH 7.0 moiety, 6-Carboxyrhodamine 6G, hydrochloride moiety, Bodipy R6G SE moiety, BODIPY R6G MeOH moiety, 6 JOE moiety, Cascade Yellow moiety, mBanana moiety, Alexa 532 moiety, Erythrosin-5-isothiocyanate pH 9.0 moiety, 6-HEX, SE pH 9.0 moiety, mOrange moiety, mHoneydew moiety, Cy 3 moiety, Rhodamine B moiety, DiI moiety, 5-TAMRA-MeOH moiety, Alexa 555 moiety, DyLight 549 moiety, BODIPY TMR-X, SE moiety, BODIPY TMR-X MeOH moiety, PO-PRO-3-DNA moiety, PO-PRO-3 moiety, Rhodamine moiety, POPO-3 moiety, Alexa 546 moiety, Calcium Orange Ca2+ moiety, TRITC moiety, Calcium Orange moiety, Rhodaminephalloidin pH 7.0 moiety, MitoTracker Orange moiety, MitoTracker Orange MeOH moiety, Phycoerythrin moiety, Magnesium Orange moiety, R-Phycoerythrin pH 7.5 moiety, 5-TAMRA pH 7.0 moiety, 5-TAMRA moiety, Rhod-2 moiety, FM 1-43 moiety, Rhod-2 Ca2+ moiety, FM 1-43 lipid moiety, LOLO-1-DNA moiety, dTomato moiety, DsRed moiety, Dapoxyl (2-aminoethyl) sulfonamide moiety, Tetramethylrhodamine dextran pH 7.0 moiety, Fluor-Ruby moiety, Resorufin moiety, Resorufin pH 9.0 moiety, mTangerine moiety, LysoTracker Red moiety, Lissaminerhodamine moiety, Cy 3.5 moiety, Rhodamine Red-X antibody conjugate pH 8.0 moiety, Sulforhodamine 101 EtOH moiety, JC-1 pH 8.2 moiety, JC-1 moiety, mStrawberry moiety, MitoTracker Red moiety, MitoTracker Red, MeOH moiety, X-Rhod-1 Ca2+ moiety, Alexa 568 moiety, 5-ROX pH 7.0 moiety, 5-ROX (5-Carboxy-X-rhodamine, triethylammonium salt) moiety, BO-PRO-3-DNA moiety, BOPRO-3 moiety, BOBO-3-DNA moiety, Ethidium Bromide moiety, ReAsH moiety, Calcium Crimson moiety, Calcium Crimson Ca2+ moiety, mRFP moiety, mCherry moiety, HcRed moiety, DyLight 594 moiety, Ethidium homodimer-1-DNA moiety, Ethidiumhomodimer moiety, Propidium Iodide moiety, SYPRO Ruby moiety, Propidium Iodide-DNA moiety, Alexa 594 moiety, BODIPY TR-X, SE moiety, BODIPY TR-X, MeOH moiety, BODIPY TR-X phallacidin pH 7.0 moiety, Alexa Fluor 610 R-phycoerythrin streptavidin pH 7.2 moiety, YO-PRO-3-DNA moiety, Di-8 ANEPPS moiety, Di-8-ANEPPS-lipid moiety, YOYO-3-DNA moiety, Nile Red-lipid moiety, Nile Red moiety, DyLight 633 moiety, mPlum moiety, TO-PRO-3-DNA moiety, DDAO pH 9.0 moiety, Fura Red high Ca moiety, Allophycocyanin pH 7.5 moiety, APC (allophycocyanin) moiety, Nile Blue, EtOH moiety, TOTO-3-DNA moiety, Cy 5 moiety, BODIPY 650/665-X, MeOH moiety, Alexa Fluor 647 R-phycoerythrin streptavidin pH 7.2 moiety, DyLight 649 moiety, Alexa 647 moiety, Fura Red Ca2+ moiety, Atto 647 moiety, Fura Red, low Ca moiety, Carboxynaphthofluorescein pH 10.0 moiety, Alexa 660 moiety, Cy 5.5 moiety, Alexa 680 moiety, DyLight 680 moiety, Alexa 700 moiety, FM 4-64, 2% CHAPS moiety, or FM 4-64 moiety. In embodiments, the dectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-DIPHENYLOXAZOLE, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(and-6)-carboxy-2,7-dichlorofluorescein, 5-(N-hexadecanoyl)aminoeosin, 5-(N-hexadecanoyl)aminoeosin, 5-chloromethylfluorescein, 5-FAM, 5-ROX, 5-TAMRA, 5-TAMRA, 6,8-difluoro-7-hydroxy-4-methylcoumarin, 6,8-difluoro-7-hydroxy-4-methylcoumarin, 6-carboxyrhodamine 6G, 6-HEX, 6-JOE, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 8-Benzyloxy-5,7-diphenylquinoline, 9,10-Bis(Phenylethynyl)Anthracene, 9,10-Diphenylanthracene, 9-METHYLCARBAZOLE, (CS)2Ir(μ-Cl)2Ir(CS)2, AAA, Acridine Orange, Acridine Orange, Acridine Yellow, Acridine Yellow, Adams Apple Red 680, Adirondack Green 520, Alexa Fluor 350, Alexa Fluor 405, Alexa Fluor 430, Alexa Fluor 430, Alexa Fluor 480, Alexa Fluor 488, Alexa Fluor 488, Alexa Fluor 488 hydrazide, Alexa Fluor 500, Alexa Fluor 514, Alexa Fluor 532, Alexa Fluor 546, Alexa Fluor 546, Alexa Fluor 555, Alexa Fluor 555, Alexa Fluor 568, Alexa Fluor 594, Alexa Fluor 594, Alexa Fluor 594, Alexa Fluor 610, Alexa Fluor 610-R-PE, Alexa Fluor 633, Alexa Fluor 635, Alexa Fluor 647, Alexa Fluor 647, Alexa Fluor 647-R-PE, Alexa Fluor 660, Alexa Fluor 680, Alexa Fluor 680-APC, Alexa Fluor 680-R-PE, Alexa Fluor 700, Alexa Fluor 750, Alexa Fluor 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO 390, ATTO 425, ATTO 430LS, ATTO 465, ATTO 488, ATTO 490LS, ATTO 495, ATTO 514, ATTO 520, ATTO 532, ATTO 550, ATTO 565, ATTO 590, ATTO 594, ATTO 610, ATTO 620, ATTO 633, ATTO 635, ATTO 647, ATTO 647N, ATTO 655, ATTO 665, ATTO 680, ATTO 700, ATTO 725, ATTO 740, ATTO Oxa12, ATTO Rho3B, ATTO Rho6G, ATTO Rho11, ATTO Rho12, ATTO Rho13, ATTO Rho14, ATTO Rho101, ATTO Thio12, Auramine O, Azami Green, Azami Green monomeric, B-phycoerythrin, BCECF, BCECF, Bex1, Biphenyl, Birch Yellow 580, Blue-green algae, BO-PRO-1, BO-PRO-3, BOBO-1, BOBO-3, BODIPY 630 650-X, BODIPY 650/665-X, BODIPY FL, BODIPY FL, BODIPY R6G, BODIPY TMR-X, BODIPY TR-X, BODIPY TR-X Ph 7.0, BODIPY TR-X phallacidin, BODIPY-DiMe, BODIPY-Phenyl, BODIPY-TMSCC, C3-Indocyanine, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue, Cascade Yellow, Catskill Green 540, CBQCA, CellMask Orange, CellTrace BODIPY TR methyl ester, CellTrace calcein violet, CellTrace™ Far Red, CellTracker Blue, CellTracker Red CMTPX, CellTracker Violet BMQC, CF405M, CF405S, CF488A, CF543, CF555, CFP, CFSE, CF™ 350, CF™ 485, Chlorophyll A, Chlorophyll B, Chromeo 488, Chromeo 494, Chromeo 505, Chromeo 546, Chromeo 642, Citrine, Citrine, ClOH butoxy aza-BODIPY, ClOH C12 aza-BODIPY, CM-H2DCFDA, Coumarin 1, Coumarin 6, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Cumarin153, Cy2, Cy3, Cy3, Cy3.5, Cy3B, Cy3B, Cy3Cy5 ET, Cy5, Cy5, Cy5.5, Cy7, Cyanine3 NHS ester, Cyanine5 carboxylic acid, Cyanine5 NHS ester, Cyclotella meneghiniana Kutzing, CypHer5, CypHer5 pH 9.15, CyQUANT GR, CyTrak Orange, Dabcyl SE, DAF-FM, DAMC (Weiss), dansyl cadaverine, Dansyl Glycine (Dioxane), DAPI, DAPI, DAPI, DAPI, DAPI (DMSO), DAPI (H2O), Dapoxyl (2-aminoethyl)sulfonamide, DCI, DCM, DCM, DCM (acetonitrile), DCM (MeOH), DDAO, Deep Purple, di-8-ANEPPS, DiA, Dichlorotris(1,10-phenanthroline) ruthenium(II), DiClOH C12 aza-BODIPY, DiClOHbutoxy aza-BODIPY, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5, DsRed, DsRed, DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549P1, DY-549P1, DY-554, DY-555, DY-557, DY-557, DY-590, DY-590, DY-615, DY-630, DY-631, DY-633, DY-635, DY-636, DY-647, DY-649P1, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight 488, DyLight 549, DyLight 594, DyLight 633, DyLight 649, DyLight 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Eosin, Ethyl Nile Blue A, Ethyl-p-Dimethylaminobenzoate, Ethyl-p-Dimethylaminobenzoate, Eu2O3 nanoparticles, Eu (Soini), Eu(tta)3DEADIT, EvaGreen, EVOblue-30, EYFP, FAD, FITC, FITC, FlAsH (Adams), Flash Red EX, FlAsH-CCPGCC, FlAsH-CCXXCC, Fluo-3, Fluo-4, Fluo-5F, Fluorescein, Fluorescein 0.1 NaOH, Fluorescein-Dibase, fluoro-emerald, Fluorol 5G, FluoSpheres blue, FluoSpheres crimson, FluoSpheres dark red, FluoSpheres orange, FluoSpheres red, FluoSpheres yellow-green, FM4-64 in CTC, FM4-64 in SDS, FM 1-43, FM 4-64, Fort Orange 600, Fura Red, Fura Red Ca free, fura-2, Fura-2 Ca free, Gadodiamide, Gd-Dtpa-Bma, Gadodiamide, Gd-Dtpa-Bma, GelGreen™, GelRed™, H9-40, HcRedl, Hemo Red 720, HiLyte Fluor 488, HiLyte Fluor 555, HiLyte Fluor 647, HiLyte Fluor 680, HiLyte Fluor 750, HiLyte Plus 555, HiLyte Plus 647, HiLyte Plus 750, HmGFP, Hoechst 33258, Hoechst 33342, Hoechst-33258, Hoechst-33258, Hops Yellow 560, HPTS, HPTS, HPTS, HPTS, HPTS, indo-1, Indo-1 Ca free, Ir(Cn)2(acac), Ir(Cs)2(acac), IR-775 chloride, IR-806, Ir-OEP-CO—Cl, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye® 800CW Alkyne, IRDye® 800CW Azide, IRDye® 800CW Carboxylate, IRDye® 800CW DBCO, IRDye® 800CW Maleimide, IRDye® 800CW NHS Ester, IRDye® 800RS, IRDye® 800RS Carboxylate, IRDye® 800RS NHS Ester, IRDye® QC-1 Carboxylate, IRDye® QC-1 NHS Ester, Isochrysis galbana—Parke, JC-1, JC-1, JOJO-1, Jonamac Red Evitag T2, Kaede Green, Kaede Red, kusabira orange, Lake Placid 490, LDS 751, Lissamine Rhodamine (Weiss), LOLO-1, lucifer yellow CH, Lucifer Yellow CH, lucifer yellow CH, Lucifer Yellow CH Dilitium salt, Lumio Green, Lumio Red, Lumogen F Orange, Lumogen Red F300, Lumogen Red F300, LysoSensor Blue DND-192, LysoSensor Green DND-153, LysoSensor Green DND-153, LysoSensor Yellow/Blue DND-160 pH 3, LysoSensor YellowBlue DND-160, LysoTracker Blue DND-22, LysoTracker Blue DND-22, LysoTracker Green DND-26, LysoTracker Red DND-99, LysoTracker Yellow HCK-123, Macoun Red Evitag T2, Macrolex Fluorescence Red G, Macrolex Fluorescence Yellow 10GN, Macrolex Fluorescence Yellow 10GN, Magnesium Green, Magnesium Octaethylporphyrin, Magnesium Orange, Magnesium Phthalocyanine, Magnesium Phthalocyanine, Magnesium Tetramesitylporphyrin, Magnesium Tetraphenylporphyrin, malachite green isothiocyanate, Maple Red-Orange 620, Marina Blue, mBanana, mBBr, mCherry, Merocyanine 540, Methyl green, Methyl green, Methyl green, Methylene Blue, Methylene Blue, mHoneyDew, MitoTracker Deep Red 633, MitoTracker Green FM, MitoTracker Orange CMTMRos, MitoTracker Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), NADH, Naphthalene, Naphthalene, Naphthofluorescein, Naphthofluorescein, NBD-X, NeuroTrace 500525, Nilblau perchlorate, nile blue, Nile Blue, Nile Blue (EtOH), nile red, Nile Red, Nile Red, Nile red, Nileblue A, NIR1, NIR2, NIR3, NIR4, NIR820, Octaethylporphyrin, OH butoxy aza-BODIPY, OHC12 aza-BODIPY, Orange Fluorescent Protein, Oregon Green 488, Oregon Green 488 DHPE, Oregon Green 514, Oxazin1, Oxazin 750, Oxazine 1, Oxazine 170, P4-3, P-Quaterphenyl, P-Terphenyl, PA-GFP (post-activation), PA-GFP (pre-activation), Pacific Orange, Palladium(II) meso-tetraphenyl-tetrabenzoporphyrin, PdOEPK, PdTFPP, PerCP-Cy5.5, Perylene, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylenediimid, Perylne Green pH-Probe 740-5.5, Phenol, Phenylalanine, pHrodo, succinimidyl ester, Phthalocyanine, PicoGreen dsDNA quantitation reagent, Pinacyanol-Iodide, Piroxicam, Platinum(II) tetraphenyltetrabenzoporphyrin, Plum Purple, PO-PRO-1, PO-PRO-3, POPO-1, POPO-3, POPOP, Porphin, PPO, Proflavin, PromoFluor-350, PromoFluor-405, PromoFluor-415, PromoFluor-488, PromoFluor-488 Premium, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, propidium iodide, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot 525, QDot 545, QDot 565, Qdot 585, Qdot 605, Qdot 625, Qdot 655, Qdot 705, Qdot 800, QpyMe2, QSY 7, QSY 7, QSY 9, QSY 21, QSY 35, quinine, Quinine Sulfate, Quinine sulfate, R-phycoerythrin, R-phycoerythrin, ReAsH-CCPGCC, ReAsH-CCXXCC, Red Beads (Weiss), Redmond Red, Resorufin, resorufin, rhod-2, Rhodamin 700 perchlorate, rhodamine, Rhodamine 6G, Rhodamine 6G, Rhodamine 101, rhodamine 110, Rhodamine 123, rhodamine 123, Rhodamine B, Rhodamine B, Rhodamine Green, Rhodamine pH-Probe 585-7.0, Rhodamine pH-Probe 585-7.5, Rhodamine phalloidin, Rhodamine Red-X, Rhodamine Red-X, Rhodamine Tag pH-Probe 585-7.0, Rhodol Green, Riboflavin, Rose Bengal, Sapphire, SBFI, SBFI Zero Na, Scenedesmus sp., SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, Spectrum Aqua, Spectrum Blue, Spectrum FRed, Spectrum Gold, Spectrum Green, Spectrum Orange, Spectrum Red, Squarylium dye III, Stains All, Stilben derivate, Stilbene, Styryl8 perchlorate, Sulfo-Cyanine3 carboxylic acid, Sulfo-Cyanine3 carboxylic acid, Sulfo-Cyanine3 NHS ester, Sulfo-Cyanine5 carboxylic acid, Sulforhodamine 101, sulforhodamine 101, Sulforhodamine B, Sulforhodamine G, Suncoast Yellow, SuperGlo BFP, SuperGlo GFP, Surf Green EX, SYBR Gold nucleic acid gel stain, SYBR Green I, SYPRO Ruby, SYTO 9, SYTO 11, SYTO 13, SYTO 16, SYTO 17, SYTO 45, SYTO 59, SYTO 60, SYTO 61, SYTO 62, SYTO 82, SYTO RNASelect, SYTO RNASelect, SYTOX Blue, SYTOX Green, SYTOX Orange, SYTOX Red, T-Sapphire, Tb (Soini), tCO, tdTomato, Terrylen, Terrylendiimid, testdye, Tetra-t-Butylazaporphine, Tetra-t-Butylnaphthalocyanine, Tetracen, Tetrakis(o-Aminophenyl)Porphyrin, Tetramesitylporphyrin, Tetramethylrhodamine, tetramethylrhodamine, Tetraphenylporphyrin, Tetraphenylporphyrin, Texas Red, Texas Red DHPE, Texas Red-X, ThiolTracker Violet, Thionin acetate, TMRE, TO-PRO-1, TO-PRO-3, Toluene, Topaz (Tsienl998), TOTO-1, TOTO-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC (Weiss), TRITC Dextran (Weiss), Tryptophan, Tyrosine, Vex1, Vybrant DyeCycle Green stain, Vybrant DyeCycle Orange stain, Vybrant DyeCycle Violet stain, WEGFP (post-activation), WellRED D2, WellRED D3, WellRED D4, WtGFP, WtGFP (Tsienl998), X-rhod-1, Yakima Yellow, YFP, YO-PRO-1, YO-PRO-3, YOYO-1, YoYo-1, YoYo-1 dsDNA, YoYo-1 ssDNA, YOYO-3, Zinc Octaethylporphyrin, Zinc Phthalocyanine, Zinc Tetramesitylporphyrin, Zinc Tetraphenylporphyrin, ZsGreen1, or ZsYellow1.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, propionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances. The compositions of the present invention can be delivered transdermally, by a topical route, or formulated as applicator sticks, solutions, suspensions, emulsions, gels, creams, ointments, pastes, jellies, paints, powders, and aerosols.

The term “drug” is used in accordance with its common meaning and refers to a substance which has a physiological effect (e.g., beneficial effect, is useful for treating a subject) when introduced into or to a subject (e.g., in or on the body of a subject or patient). A drug moiety is a radical of a drug.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomustine, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin Il (including recombinant interleukin II, or rlL.sub.2), interferon alfa-2a; interferon alfa-2b; interferon alfa-n1; interferon alfa-n3; interferon beta-1a; interferon gamma-1b; iproplatin; irnnotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol™ (i.e., paclitaxel), Taxotere™, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, lnanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “electrophilic” as used herein refers to a chemical group that is capable of accepting electron density. An “electrophilic substituent,” “electrophilic chemical moiety,” or “electrophic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “electron-withdrawing group” is used in accordance with its ordinary meaning in organic chemistry and refers to an atom or group that draws electrons from neighboring atoms (e.g., aryl or heteroaryl group), usually by resonance or inductive effects. Examples of electron-withdrawing groups include, but are not limited to, halogen, —NO₂, —SO₂CF₃, —SO₃H, —CN, —CF₃, —CCl₃, —CBr₃, —CI₃, —C(O)Cl, —C(O)Br, —C(O)I, —C(O)H, —C(O)OH, and —C(O)NH₂.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, substituted or unsubstituted piperazinyl, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups are allowed to contact such that the leaving groups are sufficiently proximal to react, interact or physically touch. In embodiments, the leaving groups is designed to facilitate the reaction. In embodiments, the leaving group is a substituent group.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-fluorenylmethyloxycarbonyl (FMOC), acetyl, benzoyl, benzyl, carbamate, p-methoxybenzyl ether (PMB), and tosyl (Ts). In embodiments, the protecting group is —PO₃H or —SO₃H. In embodiments, the protecting group is a substituent group.

The terms “polypeptide,” “peptide,” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide, or a cell is “recombinant” when it is artificial or engineered, or derived from or contains an artificial or engineered protein or nucleic acid (e.g., non-natural or not wild-type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease. In embodiments, treating is preventing. In embodiments, treating does not include preventing. In embodiments, the treating or treatment is not prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition). An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

The term “modulator” refers to a composition that increases or decreases the level of a target molecule or the function of a target molecule or the physical state of the target of the molecule relative to the absence of the composition.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer.

The term “associated” or “associated with” in the context of a substance or substance activity or function associated with a disease means that the disease is caused by (in whole or in part), or a symptom of the disease is caused by (in whole or in part) the substance or substance activity or function.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

A “therapeutic agent” as used herein refers to an agent (e.g., compound or composition described herein) that when administered to a subject will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms or the intended therapeutic effect, e.g., treatment or amelioration of an injury, disease, pathology or condition, or their symptoms including any objective or subjective parameter of treatment such as abatement; remission; diminishing of symptoms or making the injury, pathology or condition more tolerable to the patient; slowing in the rate of degeneration or decline; making the final point of degeneration less debilitating; or improving a patient's physical or mental well-being.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “tetrazine” is used in accordance with its ordinary meaning in organic chemistry and refers to an optionally substituted compound including a six-membered aromatic ring containing four nitrogen atoms with the molecular formula C₂H₂N₄. The name “tetrazine” is used in the nomenclature of derivatives of this compound. In embodiments, the tetrazine compound is a 1,2,4,5-tetrazine.

The term “dihydrotetrazine” is used in accordance with its ordinary meaning in organic chemistry and refers to a compound including a six-membered ring containing four nitrogen atoms with the molecular formula C₂H₄N₄. The name “dihydrotetrazine” is used in the nomenclature of derivatives of this compound. In embodiments, the dihydrotetrazine compound is a 1,4-dihydro-1,2,4,5-tetrazine.

The term “photocaged dihydrotetrazine” as used herein refers to a dihydrotetrazine compound covalently bonded via a linker to a photolabel moiety. In embodiments, the photolabel moiety is activated by light. In embodiments, the photolabel moiety is cleaved by light, thereby releasing a tetrazine.

The term “photolabel moiety” as used herein refers to a monovalent group capable of absorbing light. In embodiments, the light is visible light. In embodiments, the photolabel moiety is a monovalent form of a detectable agent as described herein. In embodiments, the photolabel moiety is monovalent form of BODIPY or a derivative thereof; a monovalent form of a coumarin or derivative thereof; a substituted nitrophenyl; or a substituted indolinyl. In embodiments, the photolabel moiety is not a nucleic acid (e.g., polynucleotide, oligonucleotide, DNA, or RNA) or fragment thereof.

### II. Compounds

In an aspect is provided a compound (e.g., a photocaged dihydrotetrazine compound), or a pharmaceutically acceptable salt thereof, having the formula:

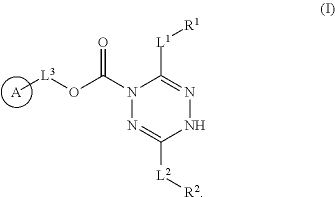

Ring A is a photolabel moiety.

L¹ is a bond or covalent linker.

L² is a bond or covalent linker.

L³ is a bond, substituted or unsubstituted alkylene (e.g., C₁-C₈, C₁-C₆, C₁-C₄, or C₁-C₂), or substituted or unsubstituted heteroalkylene (e.g., 2 to 8 membered, 2 to 6 membered, 4 to 6 membered, 2 to 3 membered, or 4 to 5 membered).

R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), or a biomolecular moiety.

R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R2D, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), substituted or unsubstituted heteroaryl (e.g., 5 to 10 membered, 5 to 9 membered, or 5 to 6 membered), or a biomolecular moiety.

R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered); R^{2A} and R^{2B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

X¹ and X² are independently —F, —Cl, —Br, or —I.

The symbols n1 and n2 are independently an integer from 0 to 4.

The symbols m1, m2, v1, and v2 are independently 1 or 2.

In embodiments, the photolabel moiety is activated by visible light. In embodiments, the visible light has a wavelength of from about 380 nm to about 700 nm. In embodiments, the visible light has a wavelength of from about 400 nm to about 600 nm. In embodiments, the visible light has a wavelength of from about 400 nm to about 530 nm. In embodiments, the visible light has a wavelength of about 380 nm. In embodiments, the visible light has a wavelength of about 390 nm. In embodiments, the visible light has a wavelength of about 400 nm. In embodiments, the visible light has a wavelength of about 405 nm. In embodiments, the visible light has a wavelength of about 415 nm. In embodiments, the visible light has a wavelength of about 425 nm. In embodiments, the visible light has a wavelength of about 435 nm. In embodiments, the visible light has a wavelength of about 445 nm. In embodiments, the visible light has a wavelength of about 450 nm. In embodiments, the visible light has a wavelength of about 460 nm. In embodiments, the visible light has a wavelength of about 470 nm. In embodiments, the visible light has a wavelength of about 480 nm. In embodiments, the visible light has a wavelength of about 490 nm. In embodiments, the visible light has a wavelength of about 500 nm. In embodiments, the visible light has a wavelength of about 510 nm. In embodiments, the visible light has a wavelength of about 520 nm. In embodiments, the visible light has a wavelength of about 525 nm. In embodiments, the visible light has a wavelength of about 530 nm. In embodiments, the visible light has a wavelength of about 540 nm. In embodiments, the visible light has a wavelength of about 550 nm. In embodiments, the visible light has a wavelength of about 560 nm. In embodiments, the visible light has a wavelength of about 570 nm. In embodiments, the visible light has a wavelength of about 580 nm. In embodiments, the visible light has a wavelength of about 590 nm. In embodiments, the visible light has a wavelength of about 600 nm. In embodiments, the visible light has a wavelength of about 625 nm. In embodiments, the visible light has a wavelength of about 650 nm. In embodiments, the visible light has a wavelength of about 675 nm. In embodiments, the visible light has a wavelength of about 700 nm.

In embodiments, the photolabel moiety is activated by visible light. In embodiments, the visible light has a wavelength of from 380 nm to 700 nm. In embodiments, the visible light has a wavelength of from 400 nm to 600 nm. In embodiments, the visible light has a wavelength of from 400 nm to 530 nm. In embodiments, the visible light has a wavelength of 380 nm. In embodiments, the visible light has a wavelength of 390 nm. In embodiments, the visible light has a wavelength of 400 nm. In embodiments, the visible light has a wavelength of 405 nm. In embodiments, the visible light has a wavelength of 415 nm. In embodiments, the visible light has a wavelength of 425 nm. In embodiments, the visible light has a wavelength of 435 nm. In embodiments, the visible light has a wavelength of 445 nm. In embodiments, the visible light has a wavelength of 450 nm. In embodiments, the visible light has a wavelength of 460 nm. In embodiments, the visible light has a wavelength of 470 nm. In embodiments, the visible light has a wavelength of 480 nm. In embodiments, the visible light has a wavelength of 490 nm. In embodiments, the visible light has a wavelength of 500 nm. In embodiments, the visible light has a wavelength of 510 nm. In embodiments, the visible light has a wavelength of 520 nm. In embodiments, the visible light has a wavelength of 525 nm. In embodiments, the visible light has a wavelength of 530 nm. In embodiments, the visible light has a wavelength of 540 nm. In embodiments, the visible light has a wavelength of 550 nm. In embodiments, the visible light has a wavelength of 560 nm. In embodiments, the visible light has a wavelength of 570 nm. In embodiments, the visible light has a wavelength of 580 nm. In embodiments, the visible light has a wavelength of 590 nm. In embodiments, the visible light has a wavelength of 600 nm. In embodiments, the visible light has a wavelength of 625 nm. In embodiments, the visible light has a wavelength of 650 nm. In embodiments, the visible light has a wavelength of 675 nm. In embodiments, the visible light has a wavelength of 700 nm.

In embodiments, the photolabel moiety is a substituted or unsubstituted aryl or substituted or unsubstituted heteroaryl. In embodiments, the photolabel moiety is an aryl or heteroaryl substituted with an electron-withdrawing group. In embodiments, the electron-withdrawing group is halogen. In embodiments, the electron-withdrawing group is —F. In embodiments, the electron-withdrawing group is —Cl. In embodiments, the electron-withdrawing group is —Br. In embodiments, the electron-withdrawing group is —I. In embodiments, the electron-withdrawing group is —NO₂. In embodiments, the electron-withdrawing group is —OCH₃. In embodiments, the electron-withdrawing group is —CN.

In embodiments, the photolabel moiety is monovalent form of BODIPY or a derivative thereof. In embodiments, the photolabel moiety is a substituted nitrophenyl. In embodiments, the photolabel moiety is a monovalent form of a coumarin or derivative thereof. In embodiments, the photolabel moiety is a substituted chromen-2-onyl. In embodiments, the photolabel moiety is a substituted indolinyl.

In embodiments, the photolabel moiety is

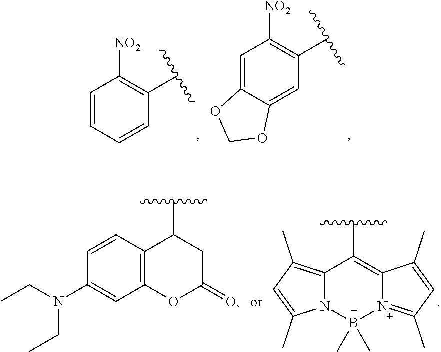

In embodiments, the photolabel moiety is

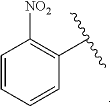 [SMILES: *c1ccccc1[N+](=O)[O-]]

In embodiments, the photolabel moiety is

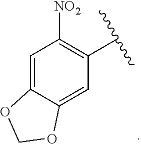 [SMILES: *c1cc2c(cc1[N+](=O)[O-])OCO2]

In embodiments, the photolabel moiety is

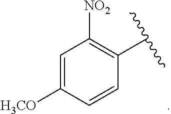 [SMILES: *c1ccc(OC)cc1[N+](=O)[O-]]

In embodiments, the photolabel moiety is

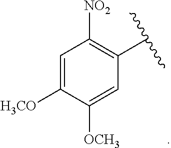 [SMILES: *c1cc(OC)c(OC)cc1[N+](=O)[O-]]

In embodiments, the photolabel moiety is

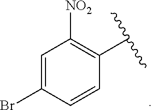 [SMILES: *c1ccc(Br)cc1[N+](=O)[O-]]

In embodiments, the photolabel moiety is

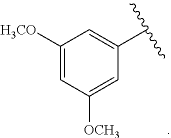 [SMILES: *c1cc(OC)cc(OC)c1]

In embodiments, the photolabel moiety is

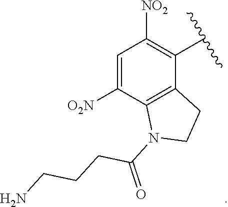 [SMILES: *c1c([N+](=O)[O-])cc([N+](=O)[O-])c2c1CCN2C(=O)CCCN]

In embodiments, the photolabel moiety is

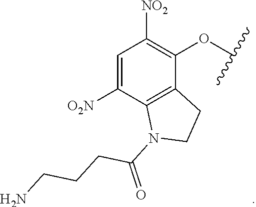 [SMILES: *Oc1c([N+](=O)[O-])cc([N+](=O)[O-])c2c1CCN2C(=O)CCCN]

In embodiments, the photolabel moiety is

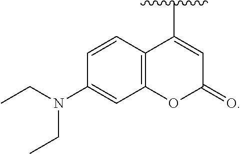 [SMILES: *c1cc(=O)oc2cc(N(CC)CC)ccc12]

In embodiments, the photolabel moiety is

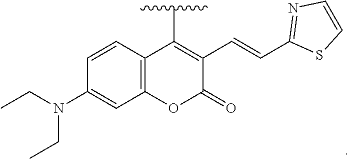 [SMILES: *c1c(/C=C/c2nccs2)c(=O)oc2cc(N(CC)CC)ccc12]

In embodiments, the photolabel moiety is

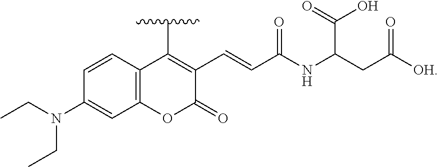 [SMILES: *c1c(/C=C/C(=O)NC(CC(=O)O)C(=O)O)c(=O)oc2cc(N(CC)CC)ccc12]

In embodiments, the photolabel moiety is

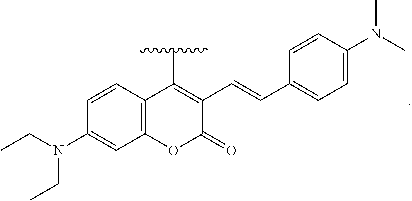 [SMILES: *c1c(/C=C/c2ccc(N(C)C)cc2)c(=O)oc2cc(N(CC)CC)ccc12]

In embodiments, the photolabel moiety is

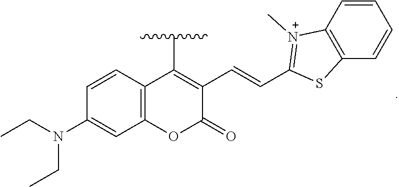 [SMILES: *c1c(/C=C/c2sc3ccccc3[n+]2C)c(=O)oc2cc(N(CC)CC)ccc12]

In embodiments, the photolabel moiety is

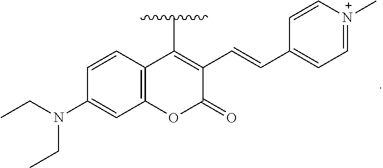 [SMILES: *c1c(/C=C/c2cc[n+](C)cc2)c(=O)oc2cc(N(CC)CC)ccc12]

In embodiments, the photolabel moiety is

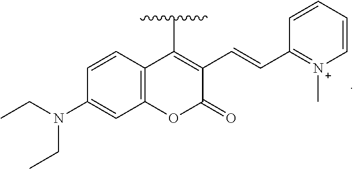 [SMILES: *c1c(/C=C/c2cccc[n+]2C)c(=O)oc2cc(N(CC)CC)ccc12]

In embodiments, the photolabel moiety is

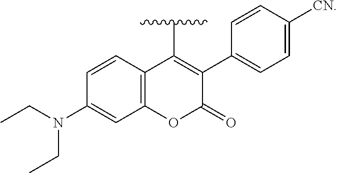 [SMILES: *c1c(-c2ccc(C#N)cc2)c(=O)oc2cc(N(CC)CC)ccc12]

In embodiments, the photolabel moiety is

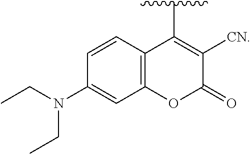 [SMILES: *c1c(C#N)c(=O)oc2cc(N(CC)CC)ccc12]

In embodiments, the photolabel moiety is

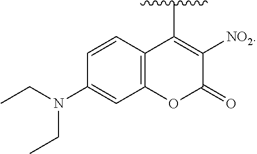 [SMILES: *c1c([N+](=O)[O-])c(=O)oc2cc(N(CC)CC)ccc12]

In embodiments, the photolabel moiety is

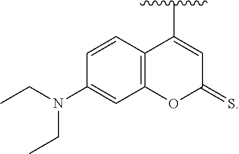 [SMILES: *c1cc(=S)oc2cc(N(CC)CC)ccc12]

In embodiments, the photolabel moiety is

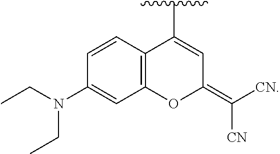 [SMILES: *C1=CC(=C(C#N)C#N)Oc2cc(N(CC)CC)ccc21]

In embodiments, the photolabel moiety is

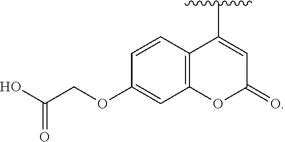 [SMILES: *c1cc(=O)oc2cc(OCC(=O)O)ccc12]

In embodiments, the photolabel moiety is

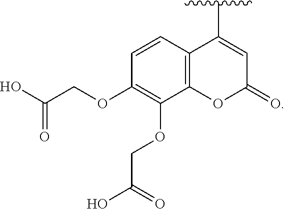 [SMILES: *c1cc(=O)oc2c(OCC(=O)O)c(OCC(=O)O)ccc12]

In embodiments, the photolabel moiety is

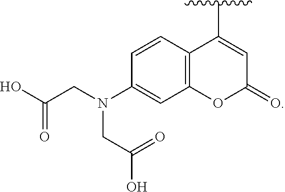 [SMILES: *c1cc(=O)oc2cc(N(CC(=O)O)CC(=O)O)ccc12]

In embodiments, the photolabel moiety is

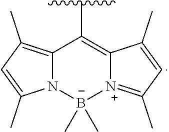 [SMILES: *C1=C2C(C)=CC(C)=[N+]2[B-](C)(C)n2c(C)cc(C)c21]

In embodiments, the photolabel moiety is

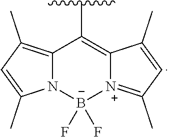 [SMILES: *C1=C2C(C)=CC(C)=[N+]2[B-](F)(F)n2c(C)cc(C)c21]

In embodiments, the photolabel moiety is

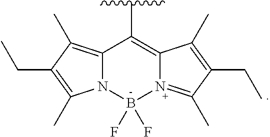

In embodiments, the photolabel moiety is

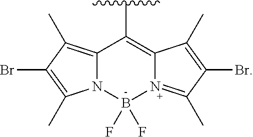

In embodiments, the photolabel moiety is

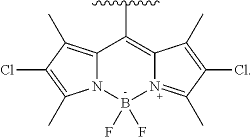

In embodiments, the photolabel moiety is

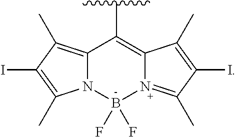

In embodiments, the photolabel moiety is

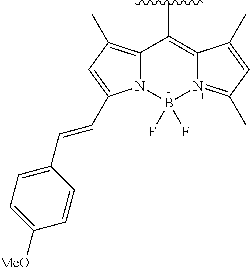

In embodiments, the photolabel moiety is

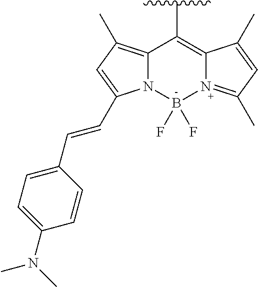

In embodiments, the photolabel moiety is

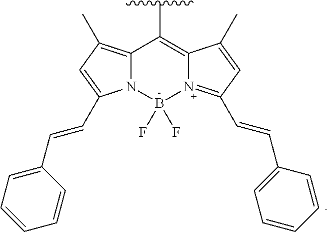

In embodiments, the photolabel moiety is

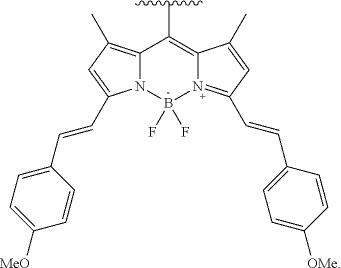

In embodiments, the photolabel moiety is

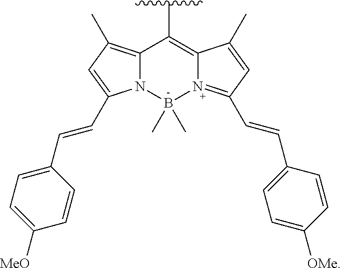

In embodiments, the photolabel moiety is

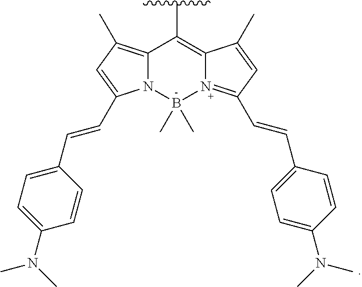

In embodiments, the photolabel moiety is

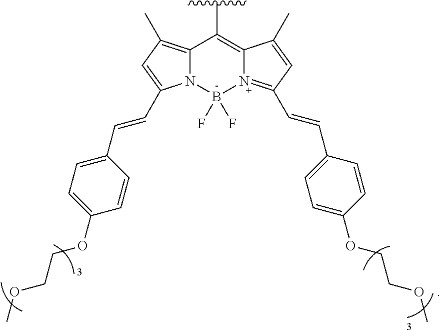

In embodiments, the photolabel moiety is

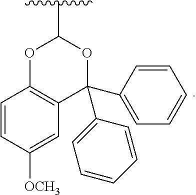 [SMILES: *C1Oc2ccc(OC)cc2C(c2ccccc2)(c2ccccc2)O1]

In embodiments, the photolabel moiety is

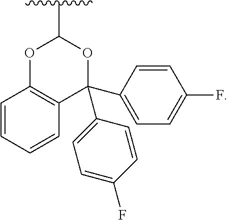 [SMILES: *C1Oc2ccccc2C(c2ccc(F)cc2)(c2ccc(F)cc2)O1]

In embodiments, the photolabel moiety is

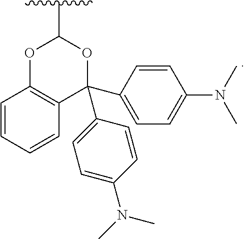 [SMILES: *C1Oc2ccccc2C(c2ccc(N(C)C)cc2)(c2ccc(N(C)C)cc2)O1]

In embodiments, the photolabel moiety is

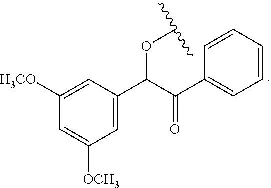 [SMILES: *OC(C(=O)c1ccccc1)c1cc(OC)cc(OC)c1]

In embodiments, the photolabel moiety is

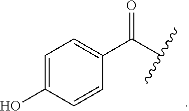 [SMILES: *C(=O)c1ccc(O)cc1]

In embodiments, the photolabel moiety is

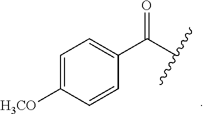 [SMILES: *C(=O)c1ccc(OC)cc1]

In embodiments, the photolabel moiety is

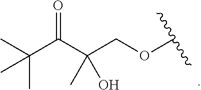 [SMILES: *OCC(C)(O)C(=O)C(C)(C)C]

In embodiments, the photolabel moiety is

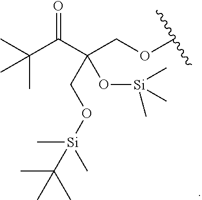 [SMILES: *OCC(CO[Si](C)(C)C(C)(C)C)(O[Si](C)(C)C)C(=O)C(C)(C)C]

In embodiments, the photolabel moiety is

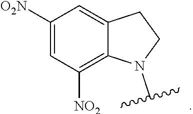 [SMILES: *N1CCc2cc([N+](=O)[O-])cc([N+](=O)[O-])c21]

In embodiments, the photolabel moiety is

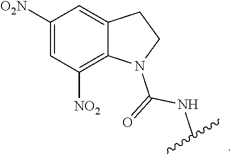 [SMILES: *NC(=O)N1CCc2cc([N+](=O)[O-])cc([N+](=O)[O-])c21]

In embodiments, the photolabel moiety is

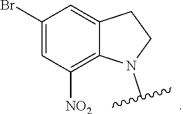 [SMILES: *N1CCc2cc(Br)cc([N+](=O)[O-])c21]

In embodiments, the photolabel moiety is

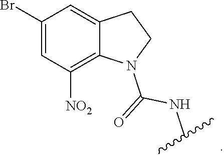 [SMILES: *NC(=O)N1CCc2cc(Br)cc([N+](=O)[O-])c21]

In embodiments, the photolabel moiety is

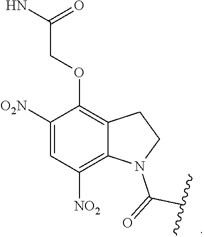 [SMILES: *C(=O)N1CCc2c(OCC(N)=O)c([N+](=O)[O-])cc([N+](=O)[O-])c21]

In embodiments, the photolabel moiety is

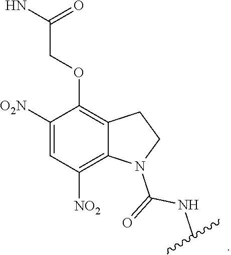 [SMILES: *NC(=O)N1CCc2c(OCC(N)=O)c([N+](=O)[O-])cc([N+](=O)[O-])c21]

In embodiments, the photolabel moeity is

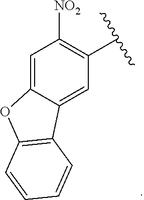 [SMILES: *c1cc2c(cc1[N+](=O)[O-])oc1ccccc12]

In embodiments, the photolabel moiety is

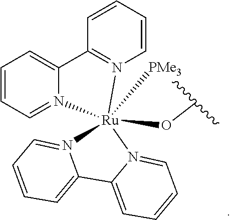

In embodiments, the photolabel moiety is

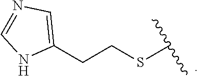 [SMILES: *SCCc1cnc[nH]1]

In embodiments, the photolabel moiety is a moiety described in Bojtár et al., Org. Lett. 2019, 21, 9410-9414, which is herein incorporated by reference for all purposes. In embodiments, the photolabel moiety is a moiety described in Bojtár et al., Org. Lett. 2019, 21, 9410-9414; Sitkowska et al., J Org. Chem. 2018, 83, 1819-1827; Peterson et al., J Am. Chem. Soc. 2018, 140, 7343-7346; Hansen et al., Chem. Soc. Rev. 2015, 44, 3358-3377; or Liu et al., Acc. Chem. Res. 2014, 47, 45-55; which are herein incorporated by reference in their entirety and for all purposes.

In embodiments, L¹ is -L¹⁰¹-L¹⁰²-L¹⁰³-L¹⁰⁴-L¹⁰⁵-.

L¹⁰¹, L¹⁰², L¹⁰³, L¹⁰⁴, and L¹⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, a substituted L¹⁰¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L¹⁰¹ is a bond. In embodiments, L¹⁰¹ is substituted or unsubstituted C₁-C₄ alkylene. In embodiments, L¹⁰¹ is unsubstituted C₁-C₄ alkylene. In embodiments, L¹⁰¹ is unsubstituted methylene. In embodiments, L¹⁰¹ is unsubstituted ethylene. In embodiments, L¹⁰¹ is unsubstituted propylene. In embodiments, L¹⁰¹ is unsubstituted n-propylene. In embodiments, L¹⁰¹ is unsubstituted isopropylene. In embodiments, L¹⁰¹ is unsubstituted butylene. In embodiments, L¹⁰¹ is unsubstituted n-butylene. In embodiments, L¹⁰¹ is unsubstituted isobutylene. In embodiments, L¹⁰¹ is unsubstituted tert-butylene. In embodiments, L¹⁰¹ is a bioconjugate linker.

In embodiments, a substituted L¹⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L¹⁰² is a bond. In embodiments, L¹⁰² is substituted or unsubstituted C₁-C₄ alkylene. In embodiments, L¹⁰² is unsubstituted C₁-C₄ alkylene. In embodiments, L¹⁰² is unsubstituted methylene. In embodiments, L¹⁰² is unsubstituted ethylene. In embodiments, L¹⁰² is unsubstituted propylene. In embodiments, L¹⁰² is unsubstituted n-propylene. In embodiments, L¹⁰² is unsubstituted isopropylene. In embodiments, L¹⁰² is unsubstituted butylene. In embodiments, L¹⁰² is unsubstituted n-butylene. In embodiments, L¹⁰² is unsubstituted isobutylene. In embodiments, L¹⁰² is unsubstituted tert-butylene. In embodiments, L¹⁰² is substituted or unsubstituted 5 to 6 membered heteroarylene. In embodiments, L¹⁰² is unsubstituted 5 to 6 membered heteroarylene. In embodiments, L¹⁰² is unsubstituted triazolylene. In embodiments, L¹⁰² is

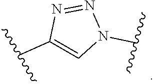 [SMILES: *c1cn(*)nn1]

In embodiments, L¹⁰² is a bioconjugate linker.

In embodiments, a substituted L¹⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L¹⁰³ is a bond. In embodiments, L¹⁰³ is substituted or unsubstituted C₁-C₄ alkylene. In embodiments, L¹⁰³ is unsubstituted C₁-C₄ alkylene. In embodiments, L¹⁰³ is unsubstituted methylene. In embodiments, L¹⁰³ is unsubstituted ethylene. In embodiments, L¹⁰³ is unsubstituted propylene. In embodiments, L¹⁰³ is unsubstituted n-propylene. In embodiments, L¹⁰³ is unsubstituted isopropylene. In embodiments, L¹⁰³ is unsubstituted butylene. In embodiments, L¹⁰³ is unsubstituted n-butylene. In embodiments, L¹⁰³ is unsubstituted isobutylene. In embodiments, L¹⁰³ is unsubstituted tert-butylene. In embodiments, L¹⁰³ is a bioconjugate linker.

In embodiments, a substituted L¹⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L¹⁰⁴ is a bond. In embodiments, L¹⁰⁴ is —C(O)NH—. In embodiments, L¹⁰⁴ is substituted or unsubstituted C₁-C₄ alkylene. In embodiments, L¹⁰⁴ is unsubstituted C₁-C₄ alkylene. In embodiments, L¹⁰⁴ is unsubstituted methylene. In embodiments, L¹⁰⁴ is unsubstituted ethylene. In embodiments, L¹⁰⁴ is unsubstituted propylene. In embodiments, L¹⁰⁴ is unsubstituted n-propylene. In embodiments, L¹⁰⁴ is unsubstituted isopropylene. In embodiments, L¹⁰⁴ is unsubstituted butylene. In embodiments, L¹⁰⁴ is unsubstituted n-butylene. In embodiments, L¹⁰⁴ is unsubstituted isobutylene. In embodiments, L¹⁰⁴ is unsubstituted tert-butylene. In embodiments, L¹⁰⁴ is substituted or unsubstituted 2 to 8 membered heteroalkylene. In embodiments, L¹⁰⁴ is

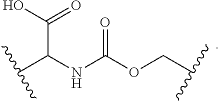 [SMILES: *COC(=O)NC(*)C(=O)O]

In embodiments, L¹⁰⁴ is a bioconjugate linker.

In embodiments, a substituted L¹⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L¹⁰⁵ is a bond. In embodiments, L¹⁰⁵ is substituted or unsubstituted C₁-C₄ alkylene. In embodiments, L¹⁰⁵ is unsubstituted C₁-C₄ alkylene. In embodiments, L¹⁰⁵ is unsubstituted methylene. In embodiments, L¹⁰⁵ is unsubstituted ethylene. In embodiments, L¹⁰⁵ is unsubstituted propylene. In embodiments, L¹⁰⁵ is unsubstituted n-propylene. In embodiments, L¹⁰⁵ is unsubstituted isopropylene. In embodiments, L¹⁰⁵ is unsubstituted butylene. In embodiments, L¹⁰⁵ is unsubstituted n-butylene. In embodiments, L¹⁰⁵ is unsubstituted isobutylene. In embodiments, L¹⁰⁵ is unsubstituted tert-butylene. In embodiments, L¹⁰⁵ is a bioconjugate linker.

In embodiments, L¹⁰¹, L¹⁰², L¹⁰³, L¹⁰⁴, and L¹⁰⁵ are a bond. In embodiments, L¹⁰¹ is a substituted or unsubstituted alkylene; L¹⁰² is a substituted or unsubstituted heteroarylene; L¹⁰³ is a substituted or unsubstituted alkylene; and L¹⁰⁴ and L¹⁰⁵ are a bond. In embodiments, L¹⁰¹ is a substituted or unsubstituted alkylene; L¹⁰² is a substituted or unsubstituted heteroarylene; L¹⁰³ is a substituted or unsubstituted alkylene; and L¹⁰⁴ is —C(O)NH—; and L¹⁰⁵ is a substituted or unsubstituted alkylene.

In embodiments, L¹ is

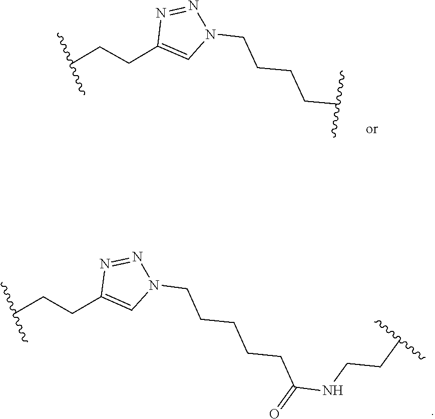 [SMILES: *CCCCn1cc(CC*)nn1 ; *CCNC(=O)CCCCCn1cc(CC*)nn1]

In embodiments, L¹ is

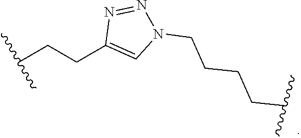 [SMILES: *CCCCn1cc(CC*)nn1]

In embodiments, L¹ is

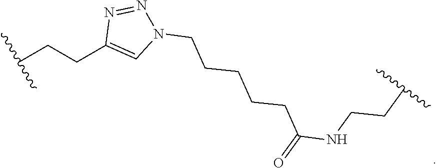 [SMILES: *CCNC(=O)CCCCCn1cc(CC*)nn1]

In embodiments, L² is -L²⁰¹-L²⁰²-L²⁰³-L²⁰⁴-L²⁰⁵-.

L²⁰¹, L²⁰², L²⁰³, L²⁰⁴, and L²⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, a substituted L²⁰¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰¹ is substituted with a plurality of groups selected from substituent groups, size-limited substituent groups, and lower substituent groups; each substituent group, size-limited substituent group, and/or lower substituent group may optionally be different. In embodiments, when L²⁰¹ is substituted, it is substituted with at least one substituent group. In embodiments, when L²⁰¹ is substituted, it is substituted with at least one size-limited substituent group. In embodiments, when L²⁰¹ is substituted, it is substituted with at least one lower substituent group.

In embodiments, L²⁰¹ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L²⁰¹ is a bond. In embodiments, L²⁰¹ is substituted or unsubstituted C₁-C₄ alkylene. In embodiments, L²⁰¹ is unsubstituted C₁-C₄ alkylene. In embodiments, L²⁰¹ is unsubstituted methylene. In embodiments, L²⁰¹ is unsubstituted ethylene. In embodiments, L²⁰¹ is unsubstituted propylene. In embodiments, L²⁰¹ is unsubstituted n-propylene. In embodiments, L²⁰¹ is unsubstituted isopropylene. In embodiments, L²⁰¹ is unsubstituted butylene. In embodiments, L²⁰¹ is unsubstituted n-butylene. In embodiments, L²⁰¹ is unsubstituted isobutylene. In embodiments, L²⁰¹ is unsubstituted tert-butylene. In embodiments, L²⁰¹ is a bioconjugate linker.

In embodiments, a substituted L²⁰² (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰² is substituted with a plurality of groups selected from substituent groups, size-limited substituent groups, and lower substituent groups; each substituent group, size-limited substituent group, and/or lower substituent group may optionally be different. In embodiments, when L²⁰² is substituted, it is substituted with at least one substituent group. In embodiments, when L²⁰² is substituted, it is substituted with at least one size-limited substituent group. In embodiments, when L²⁰² is substituted, it is substituted with at least one lower substituent group.

In embodiments, L²⁰² is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L²⁰² is a bond. In embodiments, L²⁰² is substituted or unsubstituted C₁-C₄ alkylene. In embodiments, L²⁰² is unsubstituted C₁-C₄ alkylene. In embodiments, L²⁰² is unsubstituted methylene. In embodiments, L²⁰² is unsubstituted ethylene. In embodiments, L²⁰² is unsubstituted propylene. In embodiments, L²⁰² is unsubstituted n-propylene. In embodiments, L²⁰² is unsubstituted isopropylene. In embodiments, L²⁰² is unsubstituted butylene. In embodiments, L²⁰² is unsubstituted n-butylene. In embodiments, L²⁰² is unsubstituted isobutylene. In embodiments, L²⁰² is unsubstituted tert-butylene. In embodiments, L²⁰² is substituted or unsubstituted 5 to 6 membered heteroarylene. In embodiments, L²⁰² is unsubstituted 5 to 6 membered heteroarylene. In embodiments, L²⁰² is unsubstituted triazolylene. In embodiments, L²⁰² is

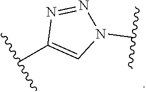 [SMILES: *c1cn(*)nn1]

In embodiments, L²⁰² is a bioconjugate linker.

In embodiments, a substituted L²⁰³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰³ is substituted with a plurality of groups selected from substituent groups, size-limited substituent groups, and lower substituent groups; each substituent group, size-limited substituent group, and/or lower substituent group may optionally be different. In embodiments, when L²⁰³ is substituted, it is substituted with at least one substituent group. In embodiments, when L²⁰³ is substituted, it is substituted with at least one size-limited substituent group. In embodiments, when L²⁰³ is substituted, it is substituted with at least one lower substituent group.

In embodiments, L²⁰³ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L²⁰³ is a bond. In embodiments, L²⁰³ is substituted or unsubstituted C₁-C₄ alkylene. In embodiments, L²⁰³ is unsubstituted C₁-C₄ alkylene. In embodiments, L²⁰³ is unsubstituted methylene. In embodiments, L²⁰³ is unsubstituted ethylene. In embodiments, L²⁰³ is unsubstituted propylene. In embodiments, L²⁰³ is unsubstituted n-propylene. In embodiments, L²⁰³ is unsubstituted isopropylene. In embodiments, L²⁰³ is unsubstituted butylene. In embodiments, L²⁰³ is unsubstituted n-butylene. In embodiments, L²⁰³ is unsubstituted isobutylene. In embodiments, L²⁰³ is unsubstituted tert-butylene. In embodiments, L²⁰³ is a bioconjugate linker.

In embodiments, a substituted L²⁰⁴ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰⁴ is substituted with a plurality of groups selected from substituent groups, size-limited substituent groups, and lower substituent groups; each substituent group, size-limited substituent group, and/or lower substituent group may optionally be different. In embodiments, when L²⁰⁴ is substituted, it is substituted with at least one substituent group. In embodiments, when L²⁰⁴ is substituted, it is substituted with at least one size-limited substituent group. In embodiments, when L²⁰⁴ is substituted, it is substituted with at least one lower substituent group.

In embodiments, L²⁰⁴ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L²⁰⁴ is a bond. In embodiments, L²⁰⁴ is —C(O)NH—. In embodiments, L²⁰⁴ is substituted or unsubstituted C₁-C₄ alkylene. In embodiments, L²⁰⁴ is unsubstituted C₁-C₄ alkylene. In embodiments, L²⁰⁴ is unsubstituted methylene. In embodiments, L²⁰⁴ is unsubstituted ethylene. In embodiments, L²⁰⁴ is unsubstituted propylene. In embodiments, L²⁰⁴ is unsubstituted n-propylene. In embodiments, L²⁰⁴ is unsubstituted isopropylene. In embodiments, L²⁰⁴ is unsubstituted butylene. In embodiments, L²⁰⁴ is unsubstituted n-butylene. In embodiments, L²⁰⁴ is unsubstituted isobutylene. In embodiments, L²⁰⁴ is unsubstituted tert-butylene. In embodiments, L²⁰⁴ is substituted or unsubstituted 2 to 8 membered heteroalkylene. In embodiments, L²⁰⁴ is

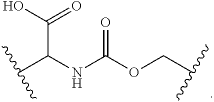 [SMILES: *COC(=O)NC(*)C(=O)O]

In embodiments, L²⁰⁴ is a bioconjugate linker.

In embodiments, a substituted L²⁰⁵ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L²⁰⁵ is substituted with a plurality of groups selected from substituent groups, size-limited substituent groups, and lower substituent groups; each substituent group, size-limited substituent group, and/or lower substituent group may optionally be different. In embodiments, when L²⁰⁵ is substituted, it is substituted with at least one substituent group. In embodiments, when L²⁰⁵ is substituted, it is substituted with at least one size-limited substituent group. In embodiments, when L²⁰⁵ is substituted, it is substituted with at least one lower substituent group.

In embodiments, L²⁰⁵ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene (e.g., C₁-C₈, C₁-C₆, C₁-C₄, or C₁-C₂), substituted or unsubstituted heteroalkylene (e.g., 2 to 8 membered, 2 to 6 membered, 4 to 6 membered, 2 to 3 membered, or 4 to 5 membered), substituted or unsubstituted cycloalkylene (e.g., C₃-C₈, C₃-C₆, C₄-C₆, or C₅-C₆), substituted or unsubstituted heterocycloalkylene (e.g., 3 to 8 membered, 3 to 6 membered, 4 to 6 membered, 4 to 5 membered, or 5 to 6 membered), substituted or unsubstituted arylene (e.g., C₆-C₁₀ or phenylene), substituted or unsubstituted heteroarylene (e.g., 5 to 10 membered, 5 to 9 membered, or 5 to 6 membered), or a bioconjugate linker.

In embodiments, L²⁰⁵ is a bond. In embodiments, L²⁰⁵ is substituted or unsubstituted C₁-C₄ alkylene. In embodiments, L²⁰⁵ is unsubstituted C₁-C₄ alkylene. In embodiments, L²⁰⁵ is unsubstituted methylene. In embodiments, L²⁰⁵ is unsubstituted ethylene. In embodiments, L²⁰⁵ is unsubstituted propylene. In embodiments, L²⁰⁵ is unsubstituted n-propylene. In embodiments, L²⁰⁵ is unsubstituted isopropylene. In embodiments, L²⁰⁵ is unsubstituted butylene. In embodiments, L²⁰⁵ is unsubstituted n-butylene. In embodiments, L²⁰⁵ is unsubstituted isobutylene. In embodiments, L²⁰⁵ is unsubstituted tert-butylene. In embodiments, L²⁰⁵ is a bioconjugate linker.

In embodiments, L²⁰¹, L²⁰², L²⁰³, L²⁰⁴, and L²⁰⁵ are a bond.

In embodiments, only one of R¹ and R² is a biomolecular moiety.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R_{1A} is substituted with a plurality of groups selected from substituent groups, size-limited substituent groups, and lower substituent groups; each substituent group, size-limited substituent group, and/or lower substituent group may optionally be different. In embodiments, when R_{1A} is substituted, it is substituted with at least one substituent group. In embodiments, when R_{1A} is substituted, it is substituted with at least one size-limited substituent group. In embodiments, when R_{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{IC} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is unsubstituted C₁-C₆ alkyl. In embodiments, R¹ is unsubstituted C₂-C₆ alkynyl. In embodiments, R¹ is

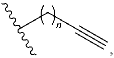

wherein n is an integer from 0 to 6. In embodiments, R¹ is

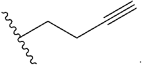 [SMILES: *CCC#C]

In embodiments, R¹ is substituted or unsubstituted cycloalkyl. In embodiments, R¹ is substituted or unsubstituted C₃-C₈ cycloalkyl. In embodiments, R¹ is unsubstituted fluorenyl. In embodiments, R¹ is unsubstituted 9-fluorenyl. In embodiments, R¹ is unsubstituted aryl or unsubstituted heteraryl. In embodiments, R¹ is unsubstituted phenyl or unsubstituted 5 to 6 membered heteraryl. In embodiments, R¹ is unsubstituted phenyl. In embodiments, R¹ is unsubstituted 5 to 6 membered heteraryl. In embodiments, R¹ is unsubstituted pyridyl. In embodiments, R¹ is unsubstituted 2-pyridyl. In embodiments, R¹ is unsubstituted 3-pyridyl. In embodiments, R¹ is unsubstituted 4-pyridyl. In embodiments, R¹ is a biomolecular moiety. In embodiments, R¹ (e.g., the biomolecular moiety) is a peptide moiety. In embodiments, R¹ (e.g., the biomolecular moiety) is a peptide moiety including from 1 to 10 amino acid residues. In embodiments, R¹ (e.g., the biomolecular moiety) is a peptide moiety including 1 amino acid residue. In embodiments, R¹ (e.g., the biomolecular moiety) is a peptide moiety including 2 amino acid residues. In embodiments, R¹ (e.g., the biomolecular moiety) is a peptide moiety including 3 amino acid residues. In embodiments, R¹ (e.g., the biomolecular moiety) is a peptide moiety including 4 amino acid residues. In embodiments, R¹ (e.g., the biomolecular moiety) is a peptide moiety including 5 amino acid residues. In embodiments, R¹(e.g., the biomolecular moiety) is a peptide moiety including 6 amino acid residues. In embodiments, R¹(e.g., the biomolecular moiety) is a peptide moiety including 7 amino acid residues. In embodiments, R¹ (e.g., the biomolecular moiety) is a peptide moiety including 8 amino acid residues. In embodiments, R¹ (e.g., the biomolecular moiety) is a peptide moiety including 9 amino acid residues. In embodiments, R¹(e.g., the biomolecular moiety) is a peptide moiety including 10 amino acid residues. In embodiments, R¹ is a peptide moiety having the sequence LKKGA (SEQ ID NO 1). In embodiments, R¹ is

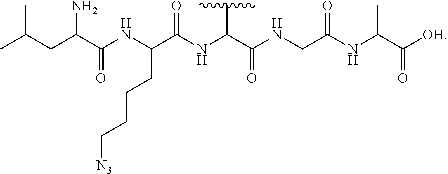 [SMILES: *C(NC(=O)C(CCCCN=[N+]=[N-])NC(=O)C(N)CC(C)C)C(=O)NCC(=O)NC(C)C(=O)O]

In embodiments, R¹ is —OP(O)(OR^{1C})(OR^{1D}); R^{1C} and R^{1D} are as described herein, including in embodiments. In embodiments, R^{1C} is hydrogen. In embodiments, R^{1C} is substituted alkyl. In embodiments, R^{1C} is substituted heteroalkyl. In embodiments, R^{1D} is hydrogen. In embodiments, R^{1D} is substituted alkyl. In embodiments, R^{1D} is substituted heteroalkyl. In embodiments, R¹ is a phosphate moiety. In embodiments, R¹ is

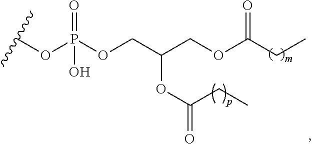

wherein m and p are independently an integer from 0 to 20. In embodiments, R¹ is

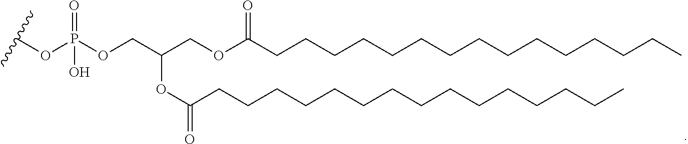 [SMILES: *OP(=O)(O)OCC(COC(=O)CCCCCCCCCCCCCCC)OC(=O)CCCCCCCCCCCCCCC]

In embodiments, n is 0. In embodiments, n is 1. In embodiments, n is 2. In embodiments, n is 3. In embodiments, n is 4. In embodiments, n is 5. In embodiments, n is 6.

In embodiments, m is 0. In embodiments, m is 1. In embodiments, m is 2. In embodiments, m is 3. In embodiments, m is 4. In embodiments, m is 5. In embodiments, m is 6. In embodiments, m is 7. In embodiments, m is 8. In embodiments, m is 9. In embodiments, m is 10. In embodiments, m is 11. In embodiments, m is 12. In embodiments, m is 13. In embodiments, m is 14. In embodiments, m is 15. In embodiments, m is 16. In embodiments, m is 17. In embodiments, m is 18. In embodiments, m is 19. In embodiments, m is 20.

In embodiments, p is 0. In embodiments, p is 1. In embodiments, p is 2. In embodiments, p is 3. In embodiments, p is 4. In embodiments, p is 5. In embodiments, p is 6. In embodiments, p is 7. In embodiments, p is 8. In embodiments, p is 9. In embodiments, p is 10. In embodiments, p is 11. In embodiments, p is 12. In embodiments, p is 13. In embodiments, p is 14. In embodiments, p is 15. In embodiments, p is 16. In embodiments, p is 17. In embodiments, p is 18. In embodiments, p is 19. In embodiments, p is 20.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R^{2B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, R² is substituted or unsubstituted phenyl or substituted or unsubstituted 5 to 6 membered heteraryl. In embodiments, R² is substituted phenyl. In embodiments, R² is phenyl substituted with halogen. In embodiments, R² is phenyl substituted with —CF₃. In embodiments, R² is phenyl substituted with —CHF₂. In embodiments, R² is phenyl substituted with —CH₂F. In embodiments, R² is phenyl substituted with —OH. In embodiments, R² is phenyl substituted with unsubstituted C₁-C₄ alkyl. In embodiments, R² is phenyl substituted with —O-(unsubstituted C₁-C₄ alkyl). In embodiments, R² is unsubstituted phenyl. In embodiments, R² is unsubstituted pyridyl. In embodiments, R² is unsubstituted 2-pyridyl. In embodiments, R² is unsubstituted 3-pyridyl. In embodiments, R² is unsubstituted 4-pyridyl. In embodiments, R² is

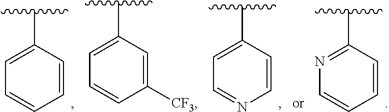

In embodiments, R² is

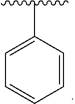 [SMILES: *c1ccccc1]

In embodiments, R² is

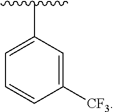 [SMILES: *c1cccc(C(F)(F)F)c1]

In embodiments, R² is

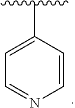 [SMILES: *c1ccncc1]

In embodiments, R² is

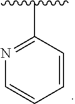 [SMILES: *c1ccccn1]

In embodiments, a substituted L³ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L³ is substituted with a plurality of groups selected from substituent groups, size-limited substituent groups, and lower substituent groups; each substituent group, size-limited substituent group, and/or lower substituent group may optionally be different. In embodiments, when L³ is substituted, it is substituted with at least one substituent group. In embodiments, when L³ is substituted, it is substituted with at least one size-limited substituent group. In embodiments, when L³ is substituted, it is substituted with at least one lower substituent group.

In embodiments, L³ is a bond, substituted or unsubstituted C₁-C₄ alkylene, or substituted or unsubstituted 2 to 6 membered heteroalkylene. In embodiments, L³ is a bond or substituted or unsubstituted C₁-C₄ alkylene. In embodiments, L³ is a bond. In embodiments, L³ is substituted or unsubstituted C₁-C₄ alkylene. In embodiments, L³ is unsubstituted C₁-C₄ alkylene. In embodiments, L³ is unsubstituted methylene. In embodiments, L³ is unsubstituted ethylene. In embodiments, L³ is unsubstituted propylene. In embodiments, L³ is unsubstituted n-propylene. In embodiments, L³ is unsubstituted isopropylene. In embodiments, L³ is unsubstituted butylene. In embodiments, L³ is unsubstituted n-butylene. In embodiments, L³ is unsubstituted isobutylene. In embodiments, L³ is unsubstituted tert-butylene. In embodiments, L³ is

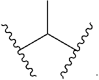 [SMILES: *C(*)C]

In embodiments, L is a substituted or unsubstituted 2 to 6 membered heteroalkylene.

In embodiments, the compound (e.g., photocaged dihydrotetrazine compound) is

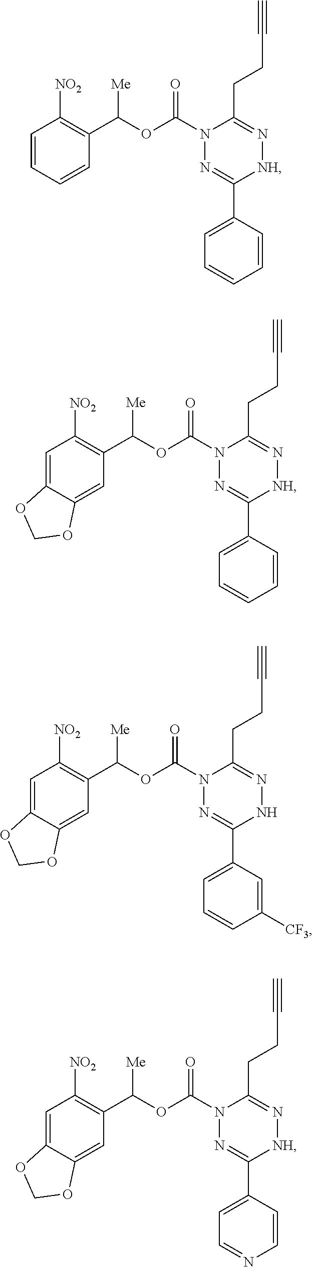

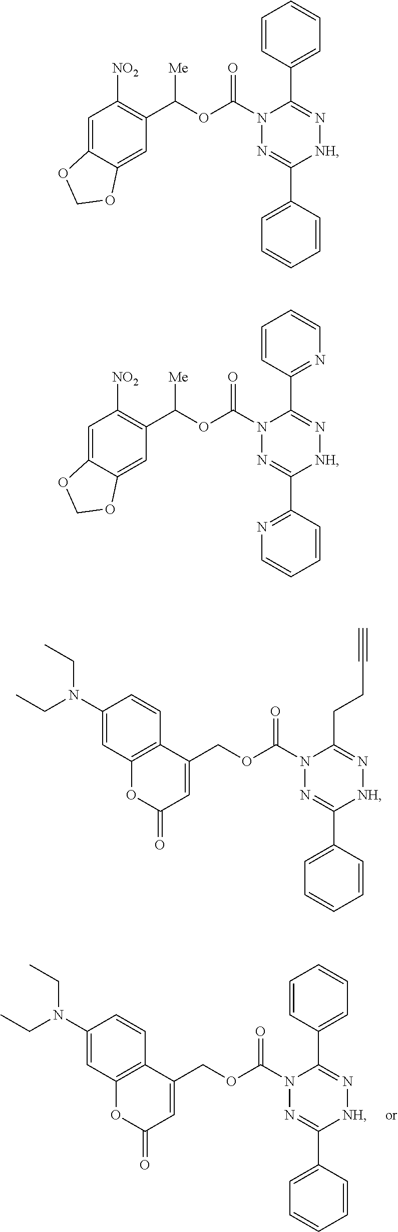

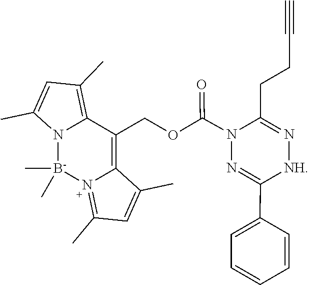

In embodiments, the compound (e.g., photocaged dihydrotetrazine compound) is

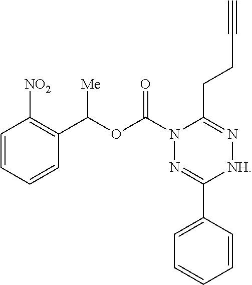 [SMILES: C#CCCC1=NNC(c2ccccc2)=NN1C(=O)OC(C)c1ccccc1[N+](=O)[O-]]

In embodiments, the compound (e.g., photocaged dihydrotetrazine compound) is

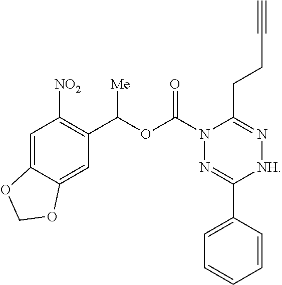 [SMILES: C#CCCC1=NNC(c2ccccc2)=NN1C(=O)OC(C)c1cc2c(cc1[N+](=O)[O-])OCO2]

In embodiments, the compound (e.g., photocaged dihydrotetrazine compound) is

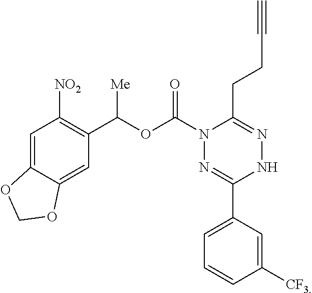 [SMILES: C#CCCC1=NNC(c2cccc(C(F)(F)F)c2)=NN1C(=O)OC(C)c1cc2c(cc1[N+](=O)[O-])OCO2]

In embodiments, the compound (e.g., photocaged dihydrotetrazine compound) is

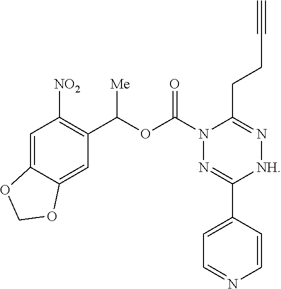 [SMILES: C#CCCC1=NNC(c2ccncc2)=NN1C(=O)OC(C)c1cc2c(cc1[N+](=O)[O-])OCO2]

In embodiments, the compound (e.g., photocaged dihydrotetrazine compound) is

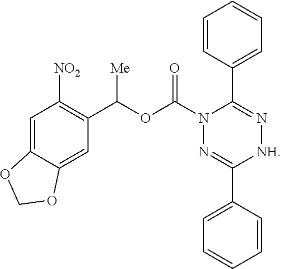 [SMILES: CC(OC(=O)N1N=C(c2ccccc2)NN=C1c1ccccc1)c1cc2c(cc1[N+](=O)[O-])OCO2]

In embodiments, the compound (e.g., photocaged dihydrotetrazine compound) is

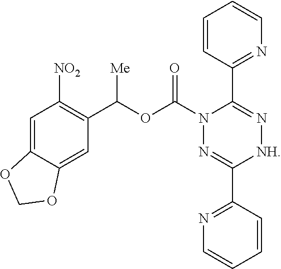 [SMILES: CC(OC(=O)N1N=C(c2ccccn2)NN=C1c1ccccn1)c1cc2c(cc1[N+](=O)[O-])OCO2]

In embodiments, the compound (e.g., photocaged dihydrotetrazine compound) is

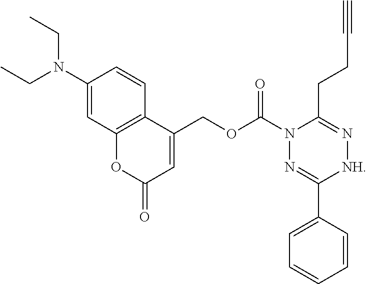 [SMILES: C#CCCC1=NNC(c2ccccc2)=NN1C(=O)OCc1cc(=O)oc2cc(N(CC)CC)ccc12]

In embodiments, the compound (e.g., photocaged dihydrotetrazine compound) is

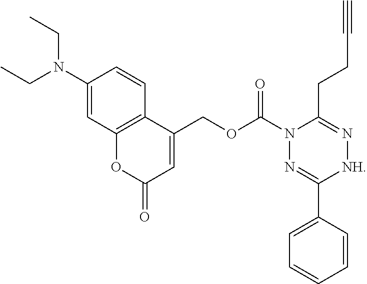 [SMILES: C#CCCC1=NNC(c2ccccc2)=NN1C(=O)OCc1cc(=O)oc2cc(N(CC)CC)ccc12]

In embodiments, the compound (e.g., photocaged dihydrotetrazine compound) is

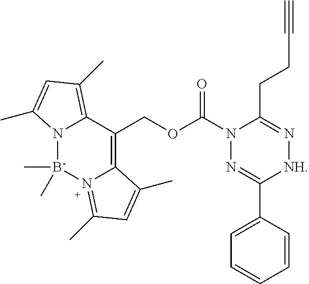

In embodiments, the compound (e.g., photocaged dihydrotetrazine compound) is

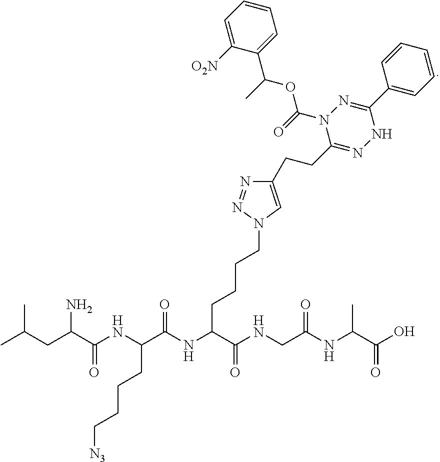 [SMILES: CC(C)CC(N)C(=O)NC(CCCCN=[N+]=[N-])C(=O)NC(CCCCn1cc(CCC2=NNC(c3ccccc3)=NN2C(=O)OC(C)c2ccccc2[N+](=O)[O-])nn1)C(=O)NCC(=O)NC(C)C(=O)O ; "."]

In embodiments, the compound (e.g., photocaged dihydrotetrazine compound) is

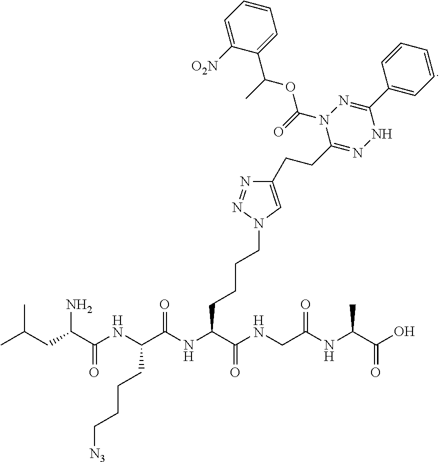 [SMILES: CC(C)C[C@H](N)C(=O)N[C@@H](CCCCN=[N+]=[N-])C(=O)N[C@@H](CCCCn1cc(CCC2=NNC(c3ccccc3)=NN2C(=O)OC(C)c2ccccc2[N+](=O)[O-])nn1)C(=O)NCC(=O)N[C@@H](C)C(=O)O ; "."]

In embodiments, the compound (e.g., photocaged dihydrotetrazine compound) is

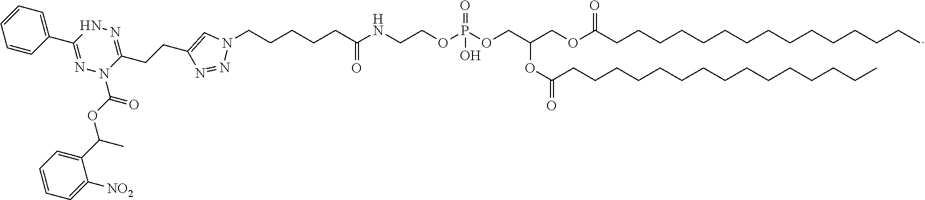 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC ; "."]

In embodiments, the compound (e.g., photocaged dihydrotetrazine compound) is

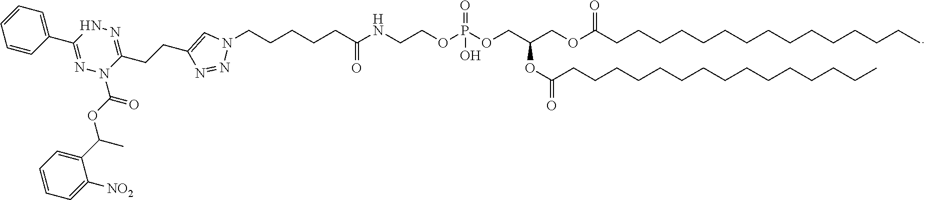 [SMILES: CCCCCCCCCCCCCCCC(=O)OC[C@H](COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC ; "."]

In embodiments, the compound (e.g., photocaged dihydrotetrazine compound) is

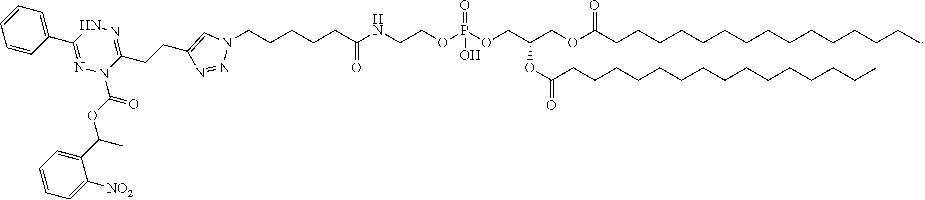 [SMILES: CCCCCCCCCCCCCCCC(=O)OC[C@@H](COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC ; "."]

In embodiments, the compound (e.g., photocaged dihydrotetrazine compound) is

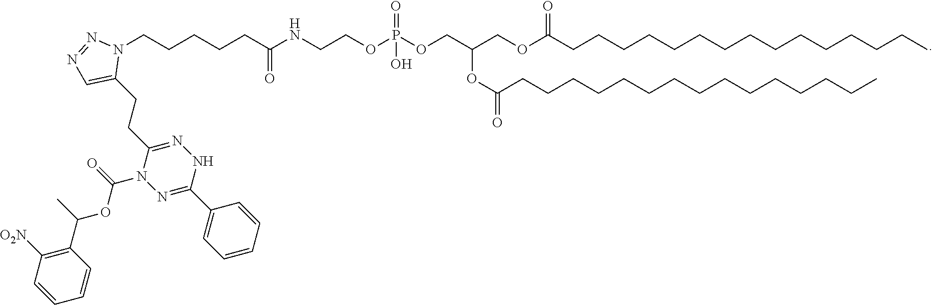 [SMILES: CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC ; "."]

In embodiments, the compound (e.g., photocaged dihydrotetrazine compound) is

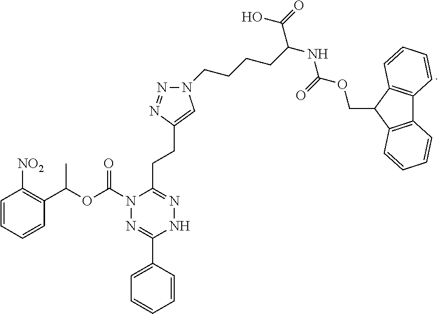 [SMILES: CC(OC(=O)N1N=C(c2ccccc2)NN=C1CCc1cn(CCCCC(NC(=O)OCC2c3ccccc3-c3ccccc32)C(=O)O)nn1)c1ccccc1[N+](=O)[O-] ; "."]

In embodiments, the compound (e.g., photocaged dihydrotetrazine compound) is

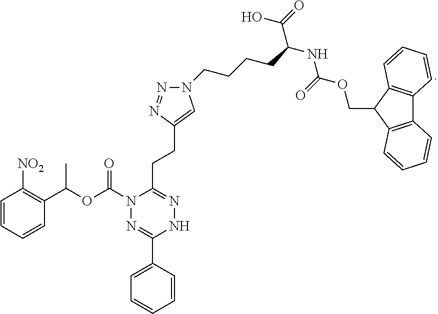 [SMILES: CC(OC(=O)N1N=C(c2ccccc2)NN=C1CCc1cn(CCCC[C@H](NC(=O)OCC2c3ccccc3-c3ccccc32)C(=O)O)nn1)c1ccccc1[N+](=O)[O-] ; "."]

In an aspect is provided a photocaged dihydrotetrazine compound. In embodiments, the photocaged tetrazine compound has the formula:

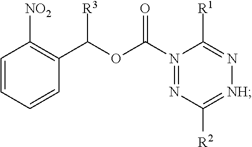

wherein R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R³ is hydrogen or substituted or unsubstituted alkyl.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R³ is hydrogen or substituted or unsubstituted alkyl (e.g., C₁-C₈, C₁-C₆, C₁-C₄, or C₁-C₂). In embodiments, R³ is hydrogen or substituted or unsubstituted C₁-C₄ alkyl. In embodiments, R³ is hydrogen. In embodiments, R³ is substituted or unsubstituted C₁-C₄ alkyl. In embodiments, R³ is unsubstituted C₁-C₄ alkyl. In embodiments, R³ is unsubstituted methyl. In embodiments, R³ is unsubstituted ethyl. In embodiments, R³ is unsubstituted propyl. In embodiments, R³ is unsubstituted n-propyl. In embodiments, R³ is unsubstituted isopropyl. In embodiments, R³ is unsubstituted butyl. In embodiments, R³ is unsubstituted n-butyl. In embodiments, R³ is unsubstituted tert-butyl.

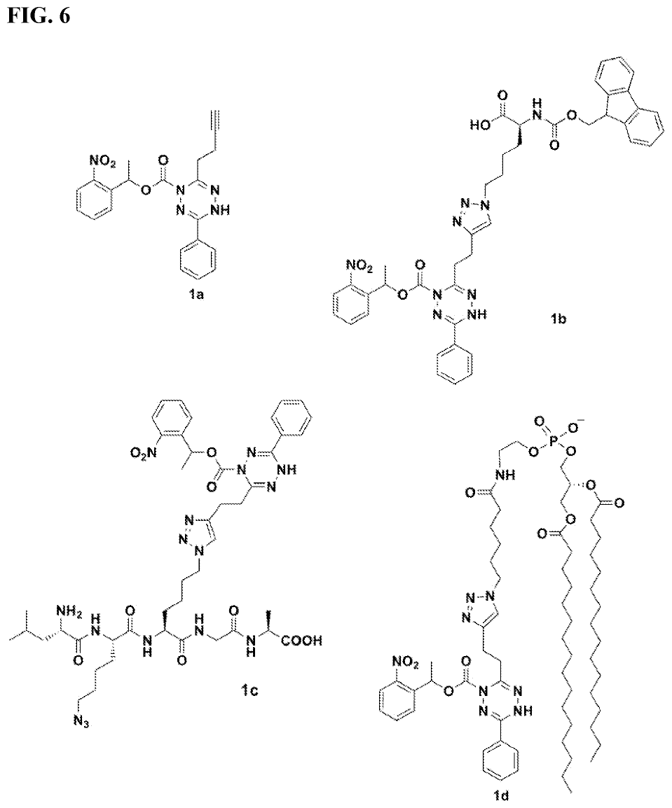

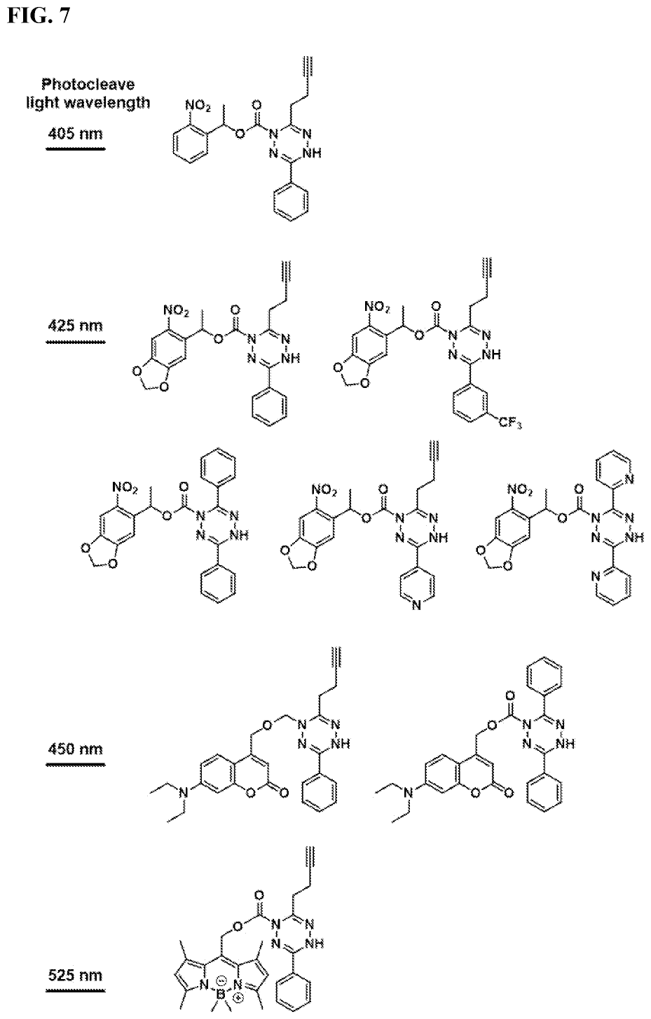

In embodiments, the photocaged dihydrotetrazine compound is a compound shown in FIG. 6. In embodiments, the photocaged dihydrotetrazine compound is a compound shown in FIG. 7.

In embodiments, the photocaged dihydrotetrazine compound is

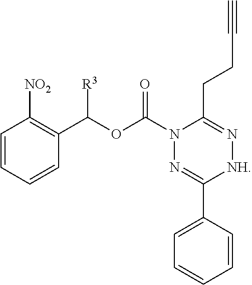

R³ is as described herein, including in embodiments.

In an aspect is provided an activated tetrazine compound. In embodiments, the activated tetrazine compound has the formula:

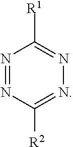

R¹ and R² are as described herein, including in embodiments.

In embodiments, the activated tetrazine compound has the formula:

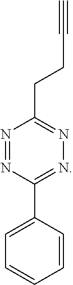 [SMILES: C#CCCc1nnc(-c2ccccc2)nn1]

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}, R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2} and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.22}, and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2B}, R^{2B.1}, R^{2B.2}, and R^{2B.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A.1}, R^{2A.2}, and R^{2A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2A.1}, R^{2A.2}, and R^{2A.3}, respectively.

In embodiments, when R^{2A} and R^{2B} substituents bonded to the same nitrogen atom are optionally joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B.1}, R^{2B.2}, and R^{2B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{2B.1}, R^{2B.2}, and R^{2B}3, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2C}, R^{2C.1}, R^{2C.2}, and R^{2C.3}, respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{2D}, R^{2D.1}, R^{2D.2}, and R^{2D.3}, respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R³, R^{3.1}, R^{3.2}, and R^{3.3}, respectively.

In embodiments, when L³ is substituted, L³ is substituted with one or more first substituent groups denoted by R^{L3.1} as explained in the definitions section above in the description of “first substituent group(s)”. In embodiments, when an R^{L3.1} substituent group is substituted, the R^{L3.1} substituent group is substituted with one or more second substituent groups denoted by R^{L3.2} as explained in the definitions section above in the description of “first substituent group(s)”. In embodiments, when an R^{L3.2} substituent group is substituted, the R^{L3.2} substituent group is substituted with one or more third substituent groups denoted by R^{L3.3} as explained in the definitions section above in the description of “first substituent group(s)”. In the above embodiments, L³, R^{L3.1}, R^{L3.2}, and R^{L3.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L³, R^{L3.1}, R^{L3.2}, and R^{L3.3}, respectively.

In embodiments, when L¹⁰¹ is substituted, L¹⁰¹ is substituted with one or more first substituent groups denoted by R^{L101.1} as explained in the definitions section above in the description of “first substituent group(s)”. In embodiments, when an R^{L101.1} substituent group is substituted, the R^{L101.1} substituent group is substituted with one or more second substituent groups denoted by R^{L101.2} as explained in the definitions section above in the description of “first substituent group(s)”. In embodiments, when an R^{L101.2} substituent group is substituted, the R^{L101.2} substituent group is substituted with one or more third substituent groups denoted by R^{L101.3} as explained in the definitions section above in the description of “first substituent group(s)”. In the above embodiments, L¹⁰¹, R^{L101.1}, R^{L101.2}, and R^{L101.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰¹, R^{L101.1}, R^{L101.2}, and R^{L101.3}, respectively.

In embodiments, when L¹⁰² is substituted, L¹⁰² is substituted with one or more first substituent groups denoted by R^{L102.1} as explained in the definitions section above in the description of “first substituent group(s)”. In embodiments, when an R^{L102.1} substituent group is substituted, the R^{L102.1} substituent group is substituted with one or more second substituent groups denoted by R^{L102.2} as explained in the definitions section above in the description of “first substituent group(s)”. In embodiments, when an R^{L102.2} substituent group is substituted, the R^{L102.2} substituent group is substituted with one or more third substituent groups denoted by R^{L102.3} as explained in the definitions section above in the description of “first substituent group(s)”. In the above embodiments, L¹⁰², R^{L102.1}, R^{L102.2}, and R^{L102.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰², R^{L102.1}, R^{L102.2}, and R^{L102.3}, respectively.

In embodiments, when L¹⁰³ is substituted, L¹⁰³ is substituted with one or more first substituent groups denoted by R^{L103.1} as explained in the definitions section above in the description of “first substituent group(s)”. In embodiments, when an R^{L103.1} substituent group is substituted, the R^{L103.1} substituent group is substituted with one or more second substituent groups denoted by R^{L103.2} as explained in the definitions section above in the description of “first substituent group(s)”. In embodiments, when an R^{L103.2} substituent group is substituted, the R^{L103.2} substituent group is substituted with one or more third substituent groups denoted by R^{L103.3} as explained in the definitions section above in the description of “first substituent group(s)”. In the above embodiments, L¹⁰³, R^{L103.1}, R^{L103.2}, and R^{L103.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰³, R^{L103.1}, R^{L103.2}, and R^{L103.3}, respectively.

In embodiments, when L¹⁰⁴ is substituted, L¹⁰⁴ is substituted with one or more first substituent groups denoted by R^{L104.1} as explained in the definitions section above in the description of “first substituent group(s)”. In embodiments, when an R^{L104.1} substituent group is substituted, the R^{L104.1} substituent group is substituted with one or more second substituent groups denoted by R^{L104.2} as explained in the definitions section above in the description of “first substituent group(s)”. In embodiments, when an R^{L104.2} substituent group is substituted, the R^{L104.2} substituent group is substituted with one or more third substituent groups denoted by R^{L104.3} as explained in the definitions section above in the description of “first substituent group(s)”. In the above embodiments, L¹⁰⁴, R^{L104.1}, R^{L104.2}, and R^{L104.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁴, R^{L104.1}, R^{L104.2}, and R^{L104.3}, respectively.

In embodiments, when L¹⁰⁵ is substituted, L¹⁰⁵ is substituted with one or more first substituent groups denoted by R^{L105.1} as explained in the definitions section above in the description of “first substituent group(s)”. In embodiments, when an R^{L105.1} substituent group is substituted, the R^{L105.1} substituent group is substituted with one or more second substituent groups denoted by R^{L105.2} as explained in the definitions section above in the description of “first substituent group(s)”. In embodiments, when an R^{L105.2} substituent group is substituted, the R^{L105.2} substituent group is substituted with one or more third substituent groups denoted by R^{L105.3} as explained in the definitions section above in the description of “first substituent group(s)”. In the above embodiments, L¹⁰⁵, R^{L105.1}, R^{L105.2}, and R^{L105.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L¹⁰⁵, R^{L105.1}, R^{L105.2}, and R^{L105.3}, respectively.

In embodiments, when L²⁰¹ is substituted, L²⁰¹ is substituted with one or more first substituent groups denoted by R^{L201.1} as explained in the definitions section above in the description of “first substituent group(s)”. In embodiments, when an R^{L201.1} substituent group is substituted, the R^{L201.1} substituent group is substituted with one or more second substituent groups denoted by R^{L201.2} as explained in the definitions section above in the description of “first substituent group(s)”. In embodiments, when an R^{L201.2} substituent group is substituted, the R^{L201.2} substituent group is substituted with one or more third substituent groups denoted by R^{L201.3} as explained in the definitions section above in the description of “first substituent group(s)”. In the above embodiments, L²⁰¹, R^{L201.1}, R^{L201.2}, and R^{L201.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰¹, R^{L201.1}, R^{L201.2}, and R^{L201.3}, respectively.

In embodiments, when L²⁰² is substituted, L²⁰² is substituted with one or more first substituent groups denoted by R^{L202.1} as explained in the definitions section above in the description of “first substituent group(s)”. In embodiments, when an R^{L202.1} substituent group is substituted, the R^{L202.1} substituent group is substituted with one or more second substituent groups denoted by R^{L202.2} as explained in the definitions section above in the description of “first substituent group(s)”. In embodiments, when an R^{L202.2} substituent group is substituted, the R^{L202.2} substituent group is substituted with one or more third substituent groups denoted by R^{L202.3} as explained in the definitions section above in the description of “first substituent group(s)”. In the above embodiments, L²⁰², R^{L202.1}, R^{L202.2}, and R^{L202.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰², R^{L202.1}, R^{L202.2}, and R^{L202.3}, respectively.

In embodiments, when L²⁰³ is substituted, L²⁰³ is substituted with one or more first substituent groups denoted by R^{L203.1} as explained in the definitions section above in the description of “first substituent group(s)”. In embodiments, when an R^{L203.1} substituent group is substituted, the R^{L203.1} substituent group is substituted with one or more second substituent groups denoted by R^{L203.2} as explained in the definitions section above in the description of “first substituent group(s)”. In embodiments, when an R^{L203.2} substituent group is substituted, the R^{L203.2} substituent group is substituted with one or more third substituent groups denoted by R^{L203.3} as explained in the definitions section above in the description of “first substituent group(s)”. In the above embodiments, L²⁰³, R^{L203.1}, R^{L203.2}, and R^{L203.3} have values corresponding to the values of L^{WW}, R^{LWW.1}, R^{LWW.2} and R^{LWW.3}, respectively, as explained in the definitions section above in the description of “first substituent group(s)”, wherein L^{WW}, R^{LWW.1}, R^{LWW.2}, and R^{LWW.3} are L²⁰³, R^{L203.1}, R^{L203.2}, and R^{L203.3}, respectively.

In embodiments, when L²⁰⁴ is substituted, L²⁰⁴ is substituted with one or more first substituent groups denoted by R^{L204.1} as explained in the definitions section above in the description of “first substituent group(s)”. In embodiments, when an R^{L204.1} substituent group is substituted, the R^{L204.1} substituent group is substituted with one or more second substituent groups denoted by R^{L204.2} as explained in the definitions section above in the description of “first substituent group(s)”. In embodiments, when an R^{L204.2} substituent group is substituted, the R^{L204.2} substituent group is substituted with one or more third substituent groups denoted by R^{L204.3} as explained in the definitions section above in the description of “first substituent group(s)”. In the above embodiments, L²⁰⁴, R^{L204.1}, R^{L204.2}, and R^{L204.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰⁴, R^{L204.1}, R^{L204.2}, and R^{L204.3}, respectively.

In embodiments, when L²⁰⁵ is substituted, L²⁰⁵ is substituted with one or more first substituent groups denoted by R^{L205.1} as explained in the definitions section above in the description of “first substituent group(s)”. In embodiments, when an R^{L205.1} substituent group is substituted, the R^{L205.1} substituent group is substituted with one or more second substituent groups denoted by R^{L205.2} as explained in the definitions section above in the description of “first substituent group(s)”. In embodiments, when an R^{L205.2} substituent group is substituted, the R^{L205.2} substituent group is substituted with one or more third substituent groups denoted by R^{L205.3} as explained in the definitions section above in the description of “first substituent group(s)”. In the above embodiments, L²⁰⁵, R^{L205.1}, R^{L205.2}, and R^{L205.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L²⁰⁵, R^{L205.1}, R^{L205.2}, and R^{L205.3}, respectively.

In embodiments, the compound is useful as a comparator compound. In embodiments, the comparator compound can be used to assess the activity of a test compound in an assay (e.g., an assay as described herein, for example in the examples section, figures, or tables).

In embodiments, the compound is a compound described herein (e.g., in the Compounds section, Examples Section, Methods Section, or in a claim, table, or figure).

### III. Pharmaceutical Compositions

In an aspect is provided a pharmaceutical composition including a compound described herein, or a pharmaceutically acceptable salt thereof, and a pharmaceutically acceptable excipient. In embodiments, the compound is a photocaged dihydrotetrazine compound.

In an aspect is provided a pharmaceutical composition including a compound described herein and a pharmaceutically acceptable excipient. In embodiments, the compound is a photocaged dihydrotetrazine compound.

In embodiments, the pharmaceutical composition includes an effective amount of the compound. In embodiments, the pharmaceutical composition includes a therapeutically effective amount of the compound.

In embodiments, the pharmaceutical composition includes a second agent (e.g., therapeutic agent). In embodiments, the pharmaceutical composition includes a second agent (e.g., therapeutic agent) in a therapeutically effective amount. In embodiments, the second agent is an anti-cancer agent.

### IV. Methods of Making

In an aspect is provided a method of making a tetrazine compound, said method comprising irradiating a photocaged dihydrotetrazine compound with light. In embodiments, the photocaged dihydrotetrazine compound is a compound of formula (I). In embodiments, the tetrazine compound is a compound of formula (II):

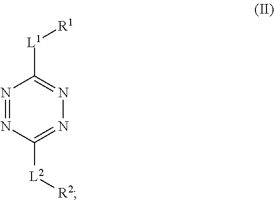

wherein L¹, R¹, L², and R² are as described herein, including in embodiments.

In embodiments, the photocaged dihydrotetrazine compound is

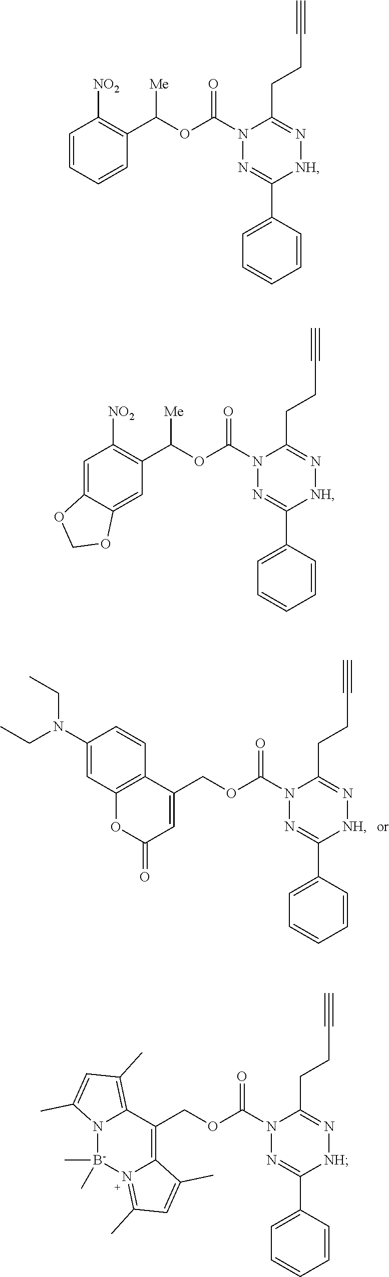

and the tetrazine compound is

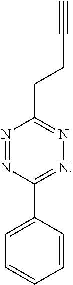 [SMILES: C#CCCc1nnc(-c2ccccc2)nn1]

In embodiments, the photocaged dihydrotetrazine compound is

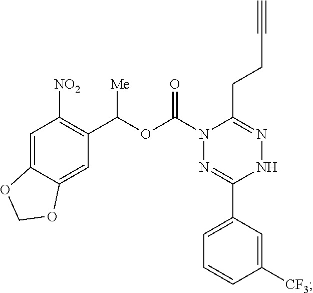 [SMILES: C#CCCC1=NNC(c2cccc(C(F)(F)F)c2)=NN1C(=O)OC(C)c1cc2c(cc1[N+](=O)[O-])OCO2]

and the tetrazine compound is

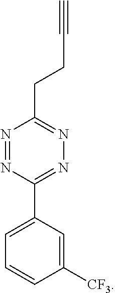 [SMILES: C#CCCc1nnc(-c2cccc(C(F)(F)F)c2)nn1]

In embodiments, the photocaged dihydrotetrazine compound is

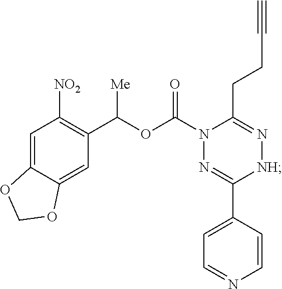 [SMILES: C#CCCC1=NNC(c2ccncc2)=NN1C(=O)OC(C)c1cc2c(cc1[N+](=O)[O-])OCO2]

and the tetrazine compound is

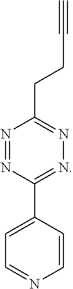 [SMILES: C#CCCc1nnc(-c2ccncc2)nn1]

In embodiments, the photocaged dihydrotetrazine compound is

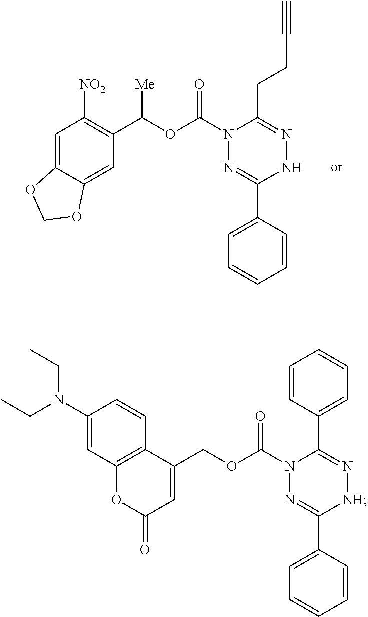 [SMILES: C#CCCC1=NNC(c2ccccc2)=NN1C(=O)OC(C)c1cc2c(cc1[N+](=O)[O-])OCO2 ; CCN(CC)c1ccc2c(COC(=O)N3N=C(c4ccccc4)NN=C3c3ccccc3)cc(=O)oc2c1]

and the tetrazine compound is

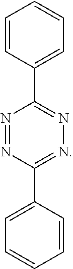 [SMILES: c1ccc(-c2nnc(-c3ccccc3)nn2)cc1]

In embodiments, the photocaged dihydrotetrazine compound is

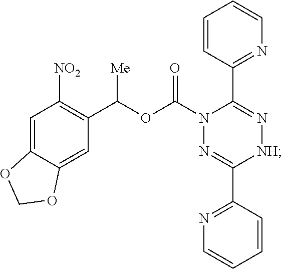 [SMILES: CC(OC(=O)N1N=C(c2ccccn2)NN=C1c1ccccn1)c1cc2c(cc1[N+](=O)[O-])OCO2]

and the tetrazine compound is

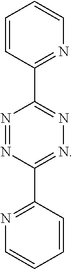 [SMILES: c1ccc(-c2nnc(-c3ccccn3)nn2)nc1]

In embodiments, the photocaged dihydrotetrazine compound is

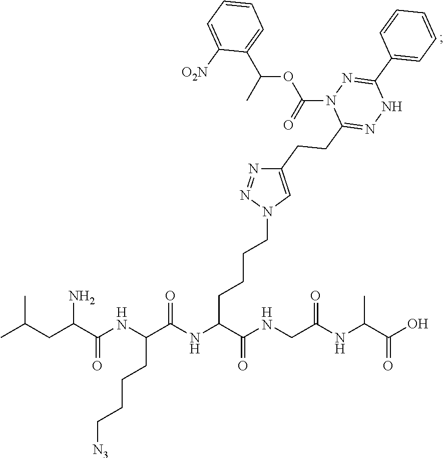 [SMILES: CC(C)CC(N)C(=O)NC(CCCCN=[N+]=[N-])C(=O)NC(CCCCn1cc(CCC2=NNC(c3ccccc3)=NN2C(=O)OC(C)c2ccccc2[N+](=O)[O-])nn1)C(=O)NCC(=O)NC(C)C(=O)O]

and the tetrazine compound is

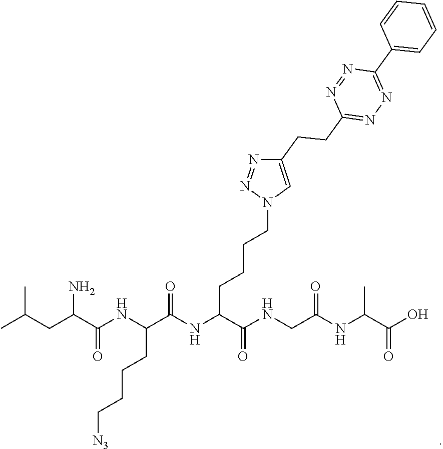 [SMILES: CC(C)CC(N)C(=O)NC(CCCCN=[N+]=[N-])C(=O)NC(CCCCn1cc(CCc2nnc(-c3ccccc3)nn2)nn1)C(=O)NCC(=O)NC(C)C(=O)O]

In embodiments, the photocaged dihydrotetrazine compound is

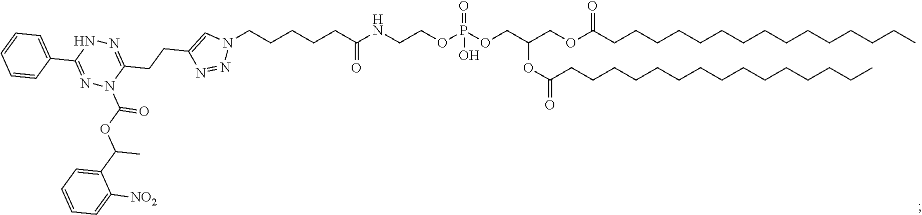 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC]

and the tetrazine compound is

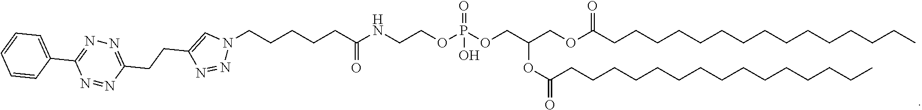 [SMILES: CCCCCCCCCCCCCCCC(=O)OCC(COP(=O)(O)OCCNC(=O)CCCCCn1cc(CCc2nnc(-c3ccccc3)nn2)nn1)OC(=O)CCCCCCCCCCCCCCC]

In embodiments, the photocaged dihydrotetrazine compound is

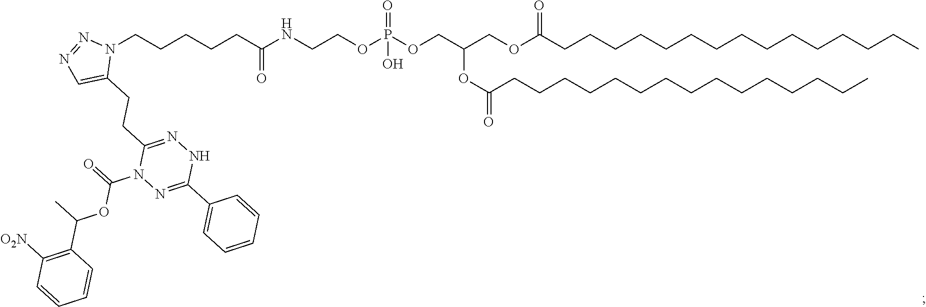 [SMILES: CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC]

and the tetrazine compound is

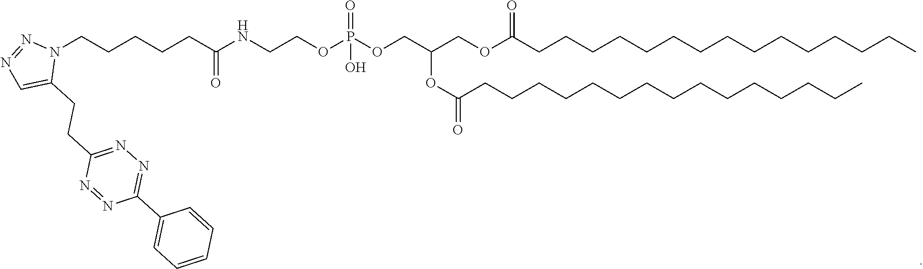 [SMILES: CCCCCCCCCCCCCCCC(=O)OCC(COP(=O)(O)OCCNC(=O)CCCCCn1nncc1CCc1nnc(-c2ccccc2)nn1)OC(=O)CCCCCCCCCCCCCCC]

In embodiments, the light is visible light. In embodiments, the light has a wavelength of from about 380 nm to about 700 nm. In embodiments, the light has a wavelength of from about 400 nm to about 600 nm. In embodiments, the light has a wavelength of from about 400 nm to about 530 nm. In embodiments, the light has a wavelength of about 380 nm. In embodiments, the light has a wavelength of about 390 nm. In embodiments, the light has a wavelength of about 400 nm. In embodiments, the light has a wavelength of about 405 nm. In embodiments, the light has a wavelength of about 415 nm. In embodiments, the light has a wavelength of about 425 nm. In embodiments, the light has a wavelength of about 435 nm. In embodiments, the light has a wavelength of about 445 nm. In embodiments, the light has a wavelength of about 450 nm. In embodiments, the light has a wavelength of about 460 nm. In embodiments, the light has a wavelength of about 470 nm. In embodiments, the light has a wavelength of about 480 nm. In embodiments, the light has a wavelength of about 490 nm. In embodiments, the light has a wavelength of about 500 nm. In embodiments, the light has a wavelength of about 510 nm. In embodiments, the light has a wavelength of about 520 nm. In embodiments, the light has a wavelength of about 525 nm. In embodiments, the light has a wavelength of about 530 nm. In embodiments, the light has a wavelength of about 540 nm. In embodiments, the light has a wavelength of about 550 nm. In embodiments, the light has a wavelength of about 560 nm. In embodiments, the light has a wavelength of about 570 nm. In embodiments, the light has a wavelength of about 580 nm. In embodiments, the light has a wavelength of about 590 nm. In embodiments, the light has a wavelength of about 600 nm. In embodiments, the light has a wavelength of about 625 nm. In embodiments, the light has a wavelength of about 650 nm. In embodiments, the light has a wavelength of about 675 nm. In embodiments, the light has a wavelength of about 700 nm.

In embodiments, the light is visible light. In embodiments, the light has a wavelength of from 380 nm to 700 nm. In embodiments, the light has a wavelength of from 400 nm to 600 nm. In embodiments, the light has a wavelength of from 400 nm to 530 nm. In embodiments, the light has a wavelength of 380 nm. In embodiments, the light has a wavelength of 390 nm. In embodiments, the light has a wavelength of 400 nm. In embodiments, the light has a wavelength of 405 nm. In embodiments, the light has a wavelength of 415 nm. In embodiments, the light has a wavelength of 425 nm. In embodiments, the light has a wavelength of 435 nm. In embodiments, the light has a wavelength of 445 nm. In embodiments, the light has a wavelength of 450 nm. In embodiments, the light has a wavelength of 460 nm. In embodiments, the light has a wavelength of 470 nm. In embodiments, the light has a wavelength of 480 nm. In embodiments, the light has a wavelength of 490 nm. In embodiments, the light has a wavelength of 500 nm. In embodiments, the light has a wavelength of 510 nm. In embodiments, the light has a wavelength of 520 nm. In embodiments, the light has a wavelength of 525 nm. In embodiments, the light has a wavelength of 530 nm. In embodiments, the light has a wavelength of 540 nm. In embodiments, the light has a wavelength of 550 nm. In embodiments, the light has a wavelength of 560 nm. In embodiments, the light has a wavelength of 570 nm. In embodiments, the light has a wavelength of 580 nm. In embodiments, the light has a wavelength of 590 nm. In embodiments, the light has a wavelength of 600 nm. In embodiments, the light has a wavelength of 625 nm. In embodiments, the light has a wavelength of 650 nm. In embodiments, the light has a wavelength of 675 nm. In embodiments, the light has a wavelength of 700 nm.

In embodiments, the method is performed in a cell. In embodiments, the cell is a mammalian cell. In embodiments, the cell is a human cell. In embodiments, the cell is a cancer cell. In embodiments, the cell is a human cancer cell. In embodiments, the cell is a cervical cancer cell. In embodiments, the cell is a human cervical cancer cell. In embodiments, the cell is a HeLa cancer cell. In embodiments, the cell is a HeLa S3 cancer cell. In embodiments, the cell is a liver cancer cell. In embodiments, the cell is a human liver cancer cell. In embodiments, the cell is a Hep 3B human liver cancer cell.

In embodiments, the method further includes reacting the tetrazine compound with a dienophile covalently linked to a monovalent form of a drug or a monovalent form of a probe. In embodiments, the dienophile covalently linked to a monovalent form of a drug or a monovalent form of a probe is a trans-cyclooctene covalently linked to a monovalent form of a drug or a monovalent form of a probe. In embodiments, the drug is an anti-cancer agent (e.g., as described herein). In embodiments, the anti-cancer agent is doxorubicin. In embodiments, the probe is a detectable agent (e.g., as described herein). In embodiments, the probe is an Alexa Fluor 488 dye. In embodiments, the probe is an Alexa Fluor 568 dye.

In embodiments, the dienophile covalently linked to a monovalent form of a drug is

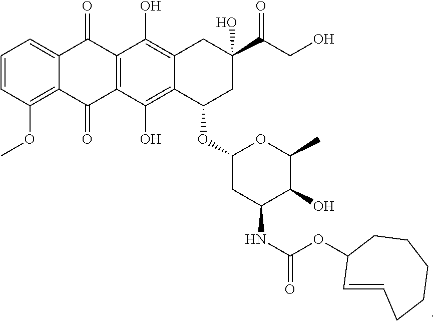 [SMILES: COc1cccc2c1C(=O)c1c(O)c3c(c(O)c1C2=O)C[C@@](O)(C(=O)CO)C[C@@H]3O[C@H]1C[C@H](NC(=O)OC2/C=C/CCCCC2)[C@H](O)[C@H](C)O1]

In embodiments, the dienophile covalently linked to a monovalent form of a probe is

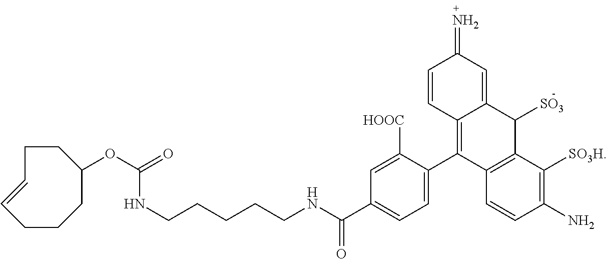

In embodiments, reacting the tetrazine compound with a dienophile covalently linked to a monovalent form of a probe forms a compound having the formula:

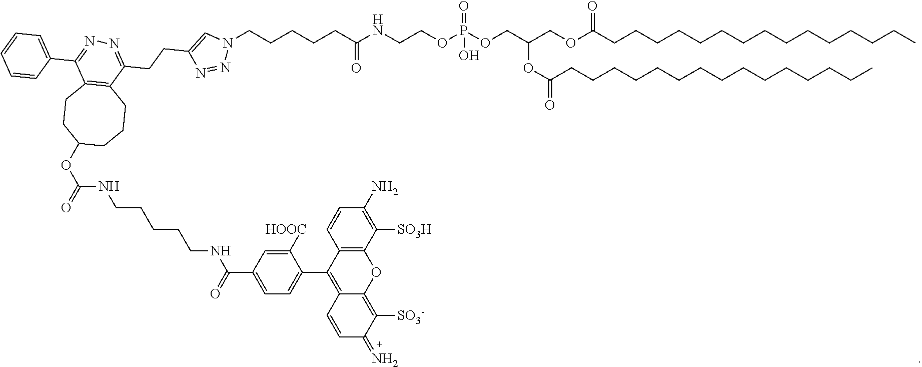

In embodiments, the dienophile covalently linked to a monovalent form of a probe is

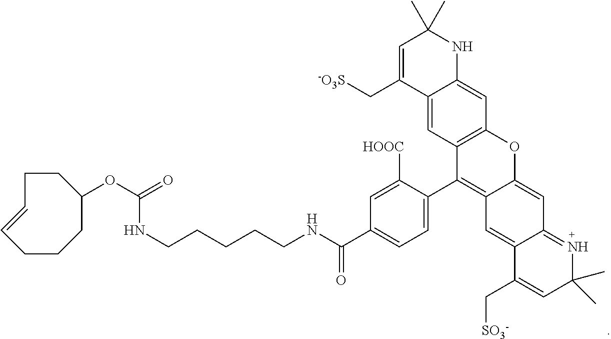

In embodiments, reacting the tetrazine compound with a dienophile covalently linked to a monovalent form of a probe forms a compound having the formula:

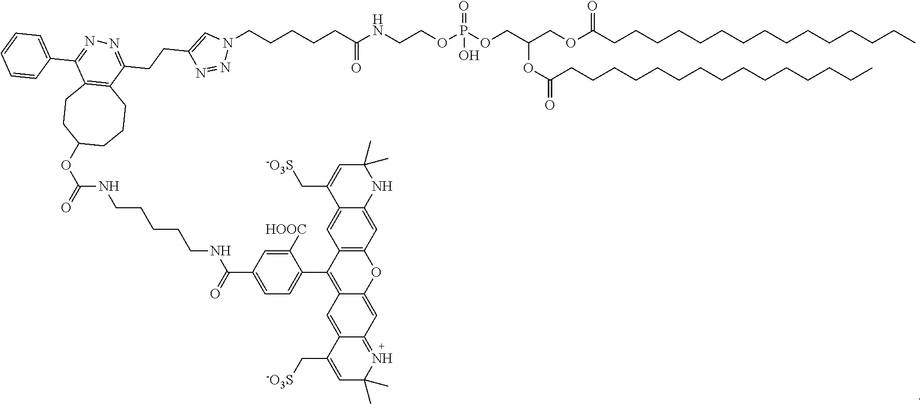

In an aspect is provided a method of making an activated tetrazine compound, said method comprising irradiating a photocaged dihydrotetrazine compound with light. In embodiments, the light is visible light. In embodiments, the visible light has a wavelength of 405 nm.

### V. Methods of Use

In an aspect is provided a method of treating a cancer in a subject in need thereof, the method including: (i) administering to the subject in need thereof a photocaged dihydrotetrazine compound or a compound described herein, or a pharmaceutically acceptable salt thereof; (ii) irradiating the photocaged dihydrotetrazine compound or the compound with light to form a tetrazine compound; and (iii) reacting the tetrazine compound with a dienophile covalently linked to a monovalent form of an anti-cancer agent, thereby releasing the anti-cancer agent.

In an aspect is provided a method of treating a cancer, the method including the steps: (i) irradiating a photocaged dihydrotetrazine compound (e.g., as described herein) with light to form an activated tetrazine (e.g., as described herein); and (ii) reacting the activated tetrazine compound with a dienophile covalently linked to a monovalent form of a drug, thereby releasing the drug.

In embodiments, the cancer is hepatocellular carcinoma. In embodiments, the cancer is cervical cancer. In embodiments, the cancer is kidney cancer. In embodiments, the cancer is skin cancer. In embodiments, the cancer is thyroid cancer, endocrine system cancer, brain cancer, breast cancer, cervix cancer, colon cancer, head and neck cancer, liver cancer, kidney cancer, lung cancer, non-small cell lung cancer, melanoma, mesothelioma, ovarian cancer, sarcoma, stomach cancer, uterus cancer, Medulloblastoma, colorectal cancer, pancreatic cancer, Hodgkin's Disease, Non-Hodgkin's Lymphoma, multiple myeloma, neuroblastoma, glioma, glioblastoma multiforme, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, colorectal cancer, papillary thyroid cancer, hepatocellular carcinoma, or prostate cancer.

In embodiments, the drug is an anti-cancer agent (e.g., as described herein). In embodiments, the drug is doxorubicin.

In embodiments, the light is visible light. In embodiments, the light has a wavelength of from about 380 nm to about 700 nm. In embodiments, the light has a wavelength of from about 400 nm to about 600 nm. In embodiments, the light has a wavelength of from about 400 nm to about 530 nm. In embodiments, the light has a wavelength of about 380 nm. In embodiments, the light has a wavelength of about 390 nm. In embodiments, the light has a wavelength of about 400 nm. In embodiments, the light has a wavelength of about 405 nm. In embodiments, the light has a wavelength of about 415 nm. In embodiments, the light has a wavelength of about 425 nm. In embodiments, the light has a wavelength of about 435 nm. In embodiments, the light has a wavelength of about 445 nm. In embodiments, the light has a wavelength of about 450 nm. In embodiments, the light has a wavelength of about 460 nm. In embodiments, the light has a wavelength of about 470 nm. In embodiments, the light has a wavelength of about 480 nm. In embodiments, the light has a wavelength of about 490 nm. In embodiments, the light has a wavelength of about 500 nm. In embodiments, the light has a wavelength of about 510 nm. In embodiments, the light has a wavelength of about 520 nm. In embodiments, the light has a wavelength of about 525 nm. In embodiments, the light has a wavelength of about 530 nm. In embodiments, the light has a wavelength of about 540 nm. In embodiments, the light has a wavelength of about 550 nm. In embodiments, the light has a wavelength of about 560 nm. In embodiments, the light has a wavelength of about 570 nm. In embodiments, the light has a wavelength of about 580 nm. In embodiments, the light has a wavelength of about 590 nm. In embodiments, the light has a wavelength of about 600 nm. In embodiments, the light has a wavelength of about 625 nm. In embodiments, the light has a wavelength of about 650 nm. In embodiments, the light has a wavelength of about 675 nm. In embodiments, the light has a wavelength of about 700 nm.

In embodiments, the light is visible light. In embodiments, the light has a wavelength of from 380 nm to 700 nm. In embodiments, the light has a wavelength of from 400 nm to 600 nm. In embodiments, the light has a wavelength of from 400 nm to 530 nm. In embodiments, the light has a wavelength of 380 nm. In embodiments, the light has a wavelength of 390 nm. In embodiments, the light has a wavelength of 400 nm. In embodiments, the light has a wavelength of 405 nm. In embodiments, the light has a wavelength of 415 nm. In embodiments, the light has a wavelength of 425 nm. In embodiments, the light has a wavelength of 435 nm. In embodiments, the light has a wavelength of 445 nm. In embodiments, the light has a wavelength of 450 nm. In embodiments, the light has a wavelength of 460 nm. In embodiments, the light has a wavelength of 470 nm. In embodiments, the light has a wavelength of 480 nm. In embodiments, the light has a wavelength of 490 nm. In embodiments, the light has a wavelength of 500 nm. In embodiments, the light has a wavelength of 510 nm. In embodiments, the light has a wavelength of 520 nm. In embodiments, the light has a wavelength of 525 nm. In embodiments, the light has a wavelength of 530 nm. In embodiments, the light has a wavelength of 540 nm. In embodiments, the light has a wavelength of 550 nm. In embodiments, the light has a wavelength of 560 nm. In embodiments, the light has a wavelength of 570 nm. In embodiments, the light has a wavelength of 580 nm. In embodiments, the light has a wavelength of 590 nm. In embodiments, the light has a wavelength of 600 nm. In embodiments, the light has a wavelength of 625 nm. In embodiments, the light has a wavelength of 650 nm. In embodiments, the light has a wavelength of 675 nm. In embodiments, the light has a wavelength of 700 nm.

In an aspect is provided a method of delivering a drug or a probe in a subject in need thereof, the method including: (i) administering to the subject in need thereof a photocaged dihydrotetrazine compound or a compound described herein, or a pharmaceutically acceptable salt thereof; (ii) irradiating the photocaged dihydrotetrazine compound or the compound with light to form a tetrazine compound; and (iii) reacting the tetrazine compound with a dienophile covalently linked to a monovalent form of the drug or a monovalent form of the probe, thereby releasing the drug or the probe.

In embodiments, the drug is an anti-cancer agent (e.g., as described herein). In embodiments, the anti-cancer agent is doxorubicin. In embodiments, the probe is a detectable agent (e.g., as described herein). In embodiments, the probe is an Alexa Fluor 488 dye. In embodiments, the probe is an Alexa Fluor 568 dye.

In an aspect is provided a method of delivering a drug or a probe, said method comprising the steps: (i) irradiating a photocaged dihydrotetrazine compound (e.g., as described herein) with light to form an activated tetrazine compound (e.g., as described herein); (ii) reacting the activated tetrazine compound with a dienophile covalently linked to a monovalent form of the drug or a monovalent form of the probe, thereby releasing the drug or the probe.

### VI. Embodiments

Embodiment P1. A method of making an activated tetrazine compound, said method comprising irradiating a photocaged dihydrotetrazine compound with light.

Embodiment P2. The method of embodiment P1, wherein the light is visible light.

Embodiment P3. A method of treating a cancer, said method comprising the steps:
(i) irradiating a photocaged dihydrotetrazine compound with light to form an activated tetrazine; and
(ii) reacting the activated tetrazine compound with a dienophile covalently linked to a monovalent form of a drug, thereby releasing the drug.

Embodiment P4. A method of delivering a drug or a probe, said method comprising the steps:
(i) irradiating a photocaged dihydrotetrazine compound with light to form an activated tetrazine compound;
(ii) reacting the activated tetrazine compound with a dienophile covalently linked to a monovalent form of the drug or a monovalent form of the probe, thereby releasing the drug or the probe.

### VII. Additional Embodiments

Embodiment 1. A method of making a tetrazine compound, said method comprising irradiating a photocaged dihydrotetrazine compound with light.

Embodiment 2. The method of embodiment 1, wherein the photocaged dihydrotetrazine compound has the formula:

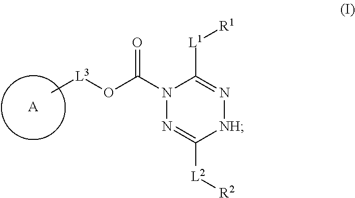

and
the tetrazine compound has the formula:

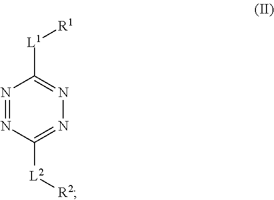

wherein
Ring A is a photolabel moiety;
L¹ is a bond or covalent linker;
L² is a bond or covalent linker;
L³ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X¹ and X² are independently —F, —Cl, —Br, or —I;
n1 and n2 are independently an integer from 0 to 4; and
m1, m2, v1, and v2 are independently 1 or 2.

Embodiment 3. The method of embodiment 2, wherein L³ is a bond or substituted or unsubstituted C₁-C₄ alkylene.

Embodiment 4. The method of embodiment 2, wherein L³ is unsubstituted C₁-C₄ alkylene.

Embodiment 5. The method of embodiment 2, wherein L³ is

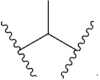 [SMILES: *C(*)C]

Embodiment 6. The method of embodiment 2, wherein L³ is unsubstituted methylene.

Embodiment 7. The method of one of embodiments 2 to 6, wherein the photolabel moiety is activated by visible light.

Embodiment 8. The method of one of embodiments 2 to 6, wherein the photolabel moiety is a substituted or unsubstituted aryl or substituted or unsubstituted heteroaryl.

Embodiment 9. The method of one of embodiments 2 to 6, wherein the photolabel moiety is

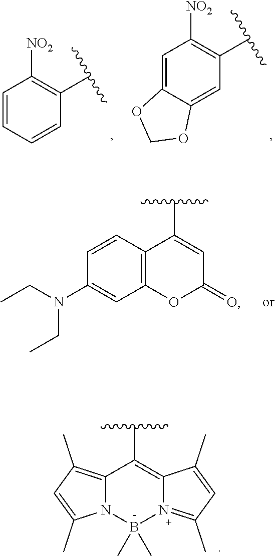

Embodiment 10. The method of one of embodiments 2 to 6, wherein L¹ is -L¹⁰¹-L¹⁰²-L¹⁰³-L¹⁰⁴-L¹⁰⁵-; and
L¹⁰¹, L¹⁰², L¹⁰³, L¹⁰⁴, and L¹⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

Embodiment 11. The method of embodiment 10, wherein L¹⁰¹, L¹⁰², L¹⁰³, L¹⁰⁴, and L¹⁰⁵ are a bond.

Embodiment 12. The method of embodiment 10, wherein
L¹⁰¹ is a substituted or unsubstituted alkylene;
L¹⁰² is a substituted or unsubstituted heteroarylene;
L¹⁰³ is a substituted or unsubstituted alkylene; and
L¹⁰⁴ and L¹⁰⁵ are a bond.

Embodiment 13. The method of embodiment 10, wherein
L¹⁰¹ is a substituted or unsubstituted alkylene;
L¹⁰² is a substituted or unsubstituted heteroarylene;
L¹⁰³ is a substituted or unsubstituted alkylene; and
L¹⁰⁴ is —C(O)NH—; and
L¹⁰⁵ is a substituted or unsubstituted alkylene.

Embodiment 14. The method of one of embodiments 2 to 9, wherein L¹ is

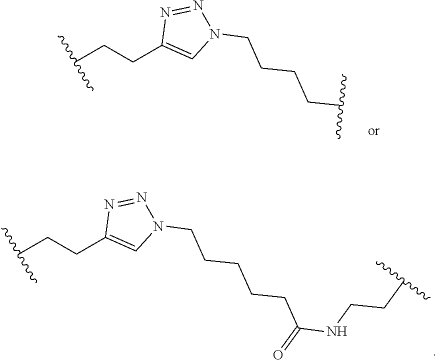 [SMILES: *CCCCn1cc(CC*)nn1 ; *CCNC(=O)CCCCCn1cc(CC*)nn1]

Embodiment 15. The method of one of embodiments 2 to 14, wherein L² is -L²⁰¹-L²⁰²-L²⁰³-L²⁰⁴-L²⁰⁵-; and
L²⁰¹, L²⁰², L²⁰³, L²⁰⁴, and L²⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

Embodiment 16. The method of embodiment 15, wherein L²⁰¹, L²⁰², L²⁰³, L²⁰⁴, and L²⁰⁵ are a bond.

Embodiment 17. The method of one of embodiments 2 to 16, wherein only one of R¹ and R² is a biomolecular moiety.

Embodiment 18. The method of one of embodiments 2 to 17, wherein R¹ is unsubstituted C₁-C₆ alkyl.

Embodiment 19. The method of embodiment 18, wherein R¹ is unsubstituted C₂-C₆ alkynyl.

Embodiment 20. The method of embodiment 19, wherein R¹ is.

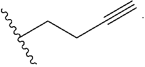 [SMILES: *CCC#C]

Embodiment 21. The method of one of embodiments 2 to 17, wherein R¹ is unsubstituted phenyl or unsubstituted 5 to 6 membered heteraryl.

Embodiment 22. The method of one of embodiments 2 to 17, wherein R¹ is unsubstituted phenyl.

Embodiment 23. The method of one of embodiments 2 to 17, wherein R¹ is unsubstituted pyridyl.

Embodiment 24. The method of one of embodiments 2 to 17, wherein R¹ is a biomolecular moiety.

Embodiment 25. The method of embodiment 24, wherein R¹ is a peptide moiety.

Embodiment 26. The method of embodiment 24, wherein R¹ is a peptide moiety having the sequence LKKGA (SEQ ID NO:1).

Embodiment 27. The method of embodiment 24, wherein R¹ is

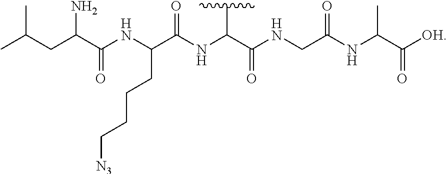 [SMILES: *C(NC(=O)C(CCCCN=[N+]=[N-])NC(=O)C(N)CC(C)C)C(=O)NCC(=O)NC(C)C(=O)O]

Embodiment 28. The method of embodiment 24, wherein R¹ is —OP(O)(OR^{1C})(OR^{1D}).

Embodiment 29. The method of embodiment 28, wherein R¹ is

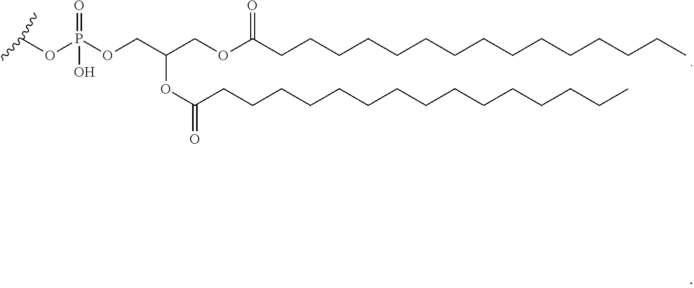 [SMILES: *OP(=O)(O)OCC(COC(=O)CCCCCCCCCCCCCCC)OC(=O)CCCCCCCCCCCCCCC]

Embodiment 30. The method of one of embodiments 2 to 29, wherein R² is substituted or unsubstituted phenyl or substituted or unsubstituted 5 to 6 membered heteraryl.

Embodiment 31. The method of embodiment 30, wherein R² is unsubstituted phenyl.

Embodiment 32. The method of embodiment 30, wherein R² is unsubstituted pyridyl.

Embodiment 33. The method of embodiment 30, wherein R² is

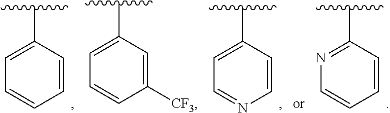

Embodiment 34. The method of embodiment 1, wherein the photocaged dihydrotetrazine compound is

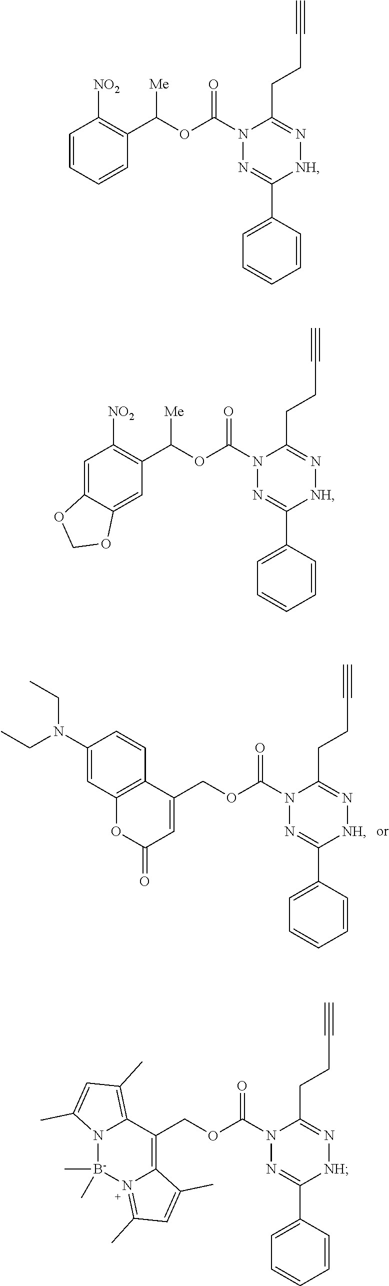

and the tetrazine compound is

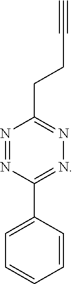 [SMILES: C#CCCc1nnc(-c2ccccc2)nn1]

Embodiment 35. The method of embodiment 1, wherein the photocaged dihydrotetrazine compound is

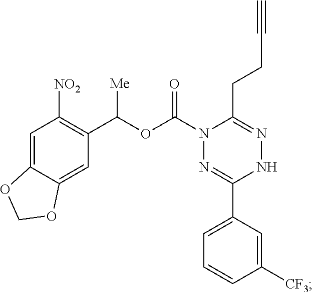 [SMILES: C#CCCC1=NNC(c2cccc(C(F)(F)F)c2)=NN1C(=O)OC(C)c1cc2c(cc1[N+](=O)[O-])OCO2]

and the tetrazine compound is

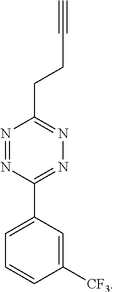 [SMILES: C#CCCc1nnc(-c2cccc(C(F)(F)F)c2)nn1]

Embodiment 36. The method of embodiment 1, wherein the photocaged dihydrotetrazine compound is

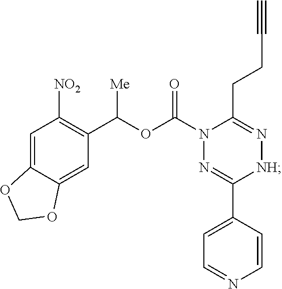 [SMILES: C#CCCC1=NNC(c2ccncc2)=NN1C(=O)OC(C)c1cc2c(cc1[N+](=O)[O-])OCO2]

and the tetrazine compound is

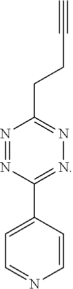 [SMILES: C#CCCc1nnc(-c2ccncc2)nn1]

Embodiment 37. The method of embodiment 1, wherein the photocaged dihydrotetrazine compound is

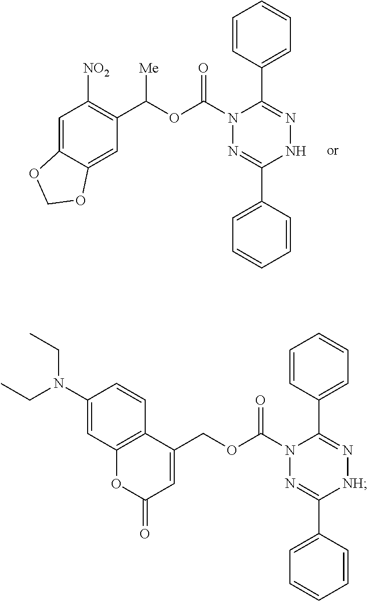 [SMILES: CC(OC(=O)N1N=C(c2ccccc2)NN=C1c1ccccc1)c1cc2c(cc1[N+](=O)[O-])OCO2 ; CCN(CC)c1ccc2c(COC(=O)N3N=C(c4ccccc4)NN=C3c3ccccc3)cc(=O)oc2c1]

and the tetrazine compound is

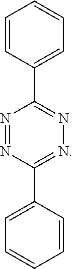 [SMILES: c1ccc(-c2nnc(-c3ccccc3)nn2)cc1]

Embodiment 38. The method of embodiment 1, wherein the photocaged dihydrotetrazine compound is

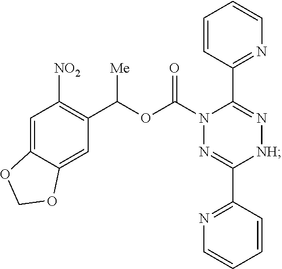 [SMILES: CC(OC(=O)N1N=C(c2ccccn2)NN=C1c1ccccn1)c1cc2c(cc1[N+](=O)[O-])OCO2]

and the tetrazine compound is

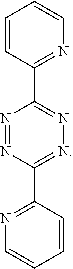 [SMILES: c1ccc(-c2nnc(-c3ccccn3)nn2)nc1]

Embodiment 39. The method of embodiment 1, wherein the photocaged dihydrotetrazine compound is

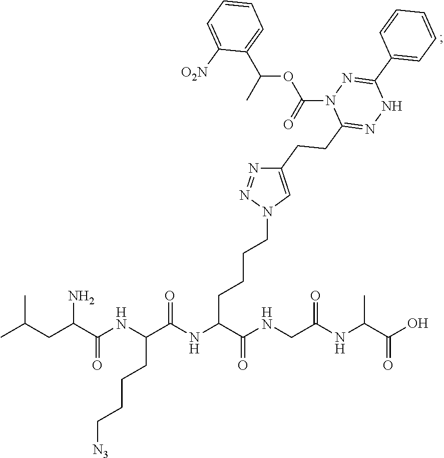 [SMILES: CC(C)CC(N)C(=O)NC(CCCCN=[N+]=[N-])C(=O)NC(CCCCn1cc(CCC2=NNC(c3ccccc3)=NN2C(=O)OC(C)c2ccccc2[N+](=O)[O-])nn1)C(=O)NCC(=O)NC(C)C(=O)O]

and the tetrazine compound is

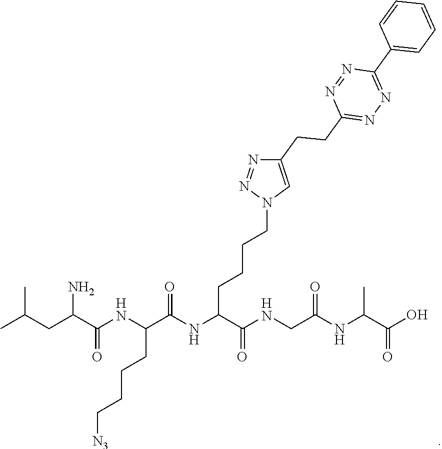 [SMILES: CC(C)CC(N)C(=O)NC(CCCCN=[N+]=[N-])C(=O)NC(CCCCn1cc(CCc2nnc(-c3ccccc3)nn2)nn1)C(=O)NCC(=O)NC(C)C(=O)O]

Embodiment 40. The method of embodiment 1, wherein the photocaged dihydrotetrazine compound is

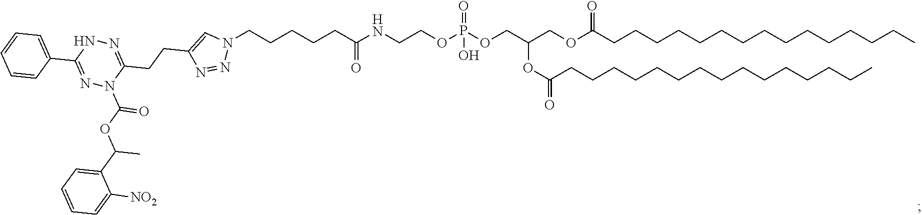 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC]

and the tetrazine compound is

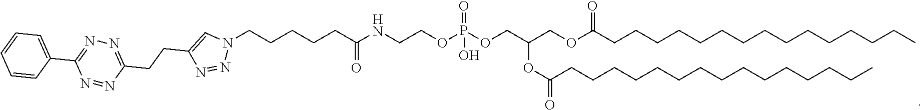 [SMILES: CCCCCCCCCCCCCCCC(=O)OCC(COP(=O)(O)OCCNC(=O)CCCCCn1cc(CCc2nnc(-c3ccccc3)nn2)nn1)OC(=O)CCCCCCCCCCCCCCC]

Embodiment 41. The method of embodiment 1, wherein the photocaged dihydrotetrazine compound is

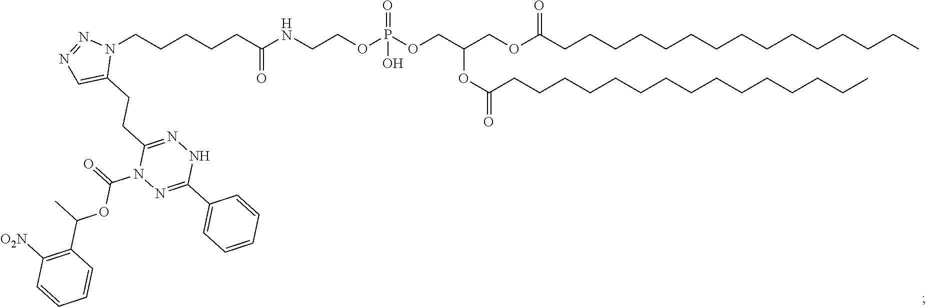 [SMILES: CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC]

and the tetrazine compound is

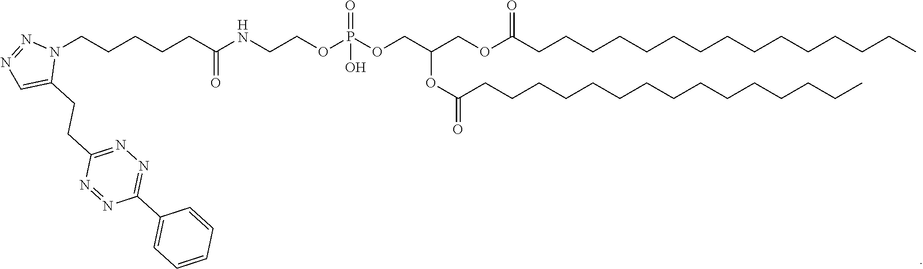 [SMILES: CCCCCCCCCCCCCCCC(=O)OCC(COP(=O)(O)OCCNC(=O)CCCCCn1nncc1CCc1nnc(-c2ccccc2)nn1)OC(=O)CCCCCCCCCCCCCCC]

Embodiment 42. The method of one of embodiments 1 to 41, wherein the light is visible light.

Embodiment 43. The method of embodiment 42, wherein the light has a wavelength of from about 380 nm to about 700 nm.

Embodiment 44. The method of embodiment 42, wherein the light has a wavelength of from about 400 nm to about 530 nm.

Embodiment 45. The method of embodiment 42, wherein the light has a wavelength of about 405 nm.

Embodiment 46. The method of embodiment 42, wherein the light has a wavelength of about 425 nm.

Embodiment 47. The method of embodiment 42, wherein the light has a wavelength of about 450 nm.

Embodiment 48. The method of embodiment 42, wherein the light has a wavelength of about 525 nm.

Embodiment 49. The method of one of embodiments 1 to 45, wherein the method is performed in a cell.

Embodiment 50. The method of one of embodiments 1 to 49, further comprising reacting the tetrazine compound with a dienophile covalently linked to a monovalent form of a drug or a monovalent form of a probe.

Embodiment 51. The method of embodiment 50, wherein the dienophile covalently linked to a monovalent form of a drug or a monovalent form of a probe is a trans-cyclooctene covalently linked to a monovalent form of a drug or a monovalent form of a probe.

Embodiment 52. The method of one of embodiments 50 to 51, wherein the drug is an anti-cancer agent.

Embodiment 53. The method of embodiment 52, wherein the anti-cancer agent is doxorubicin.

Embodiment 54. The method of one of embodiments 50 to 51, wherein the dienophile covalently linked to a monovalent form of a drug is

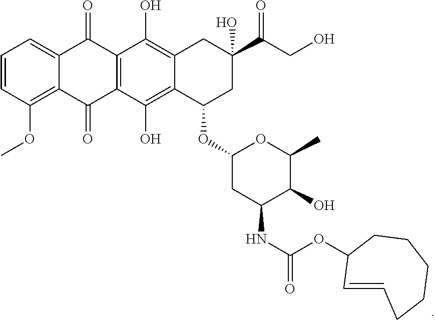 [SMILES: COc1cccc2c1C(=O)c1c(O)c3c(c(O)c1C2=O)C[C@@](O)(C(=O)CO)C[C@@H]3O[C@H]1C[C@H](NC(=O)OC2/C=C/CCCCC2)[C@H](O)[C@H](C)O1]

Embodiment 55. The method of one of embodiments 50 to 51, wherein the probe is an Alexa Fluor 488 dye.

Embodiment 56. The method of one of embodiments 50 to 51, wherein the dienophile covalently linked to a monovalent form of a probe is

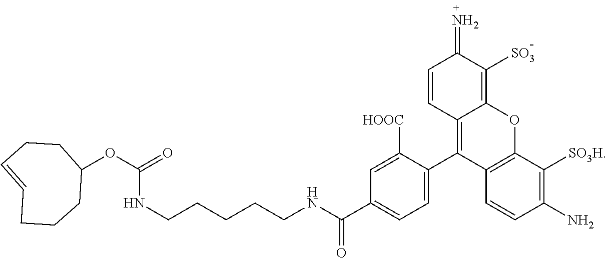

Embodiment 57. The method of one of embodiments 50 to 51, wherein the probe is an Alexa Fluor 568 dye.

Embodiment 58. A compound having the formula:

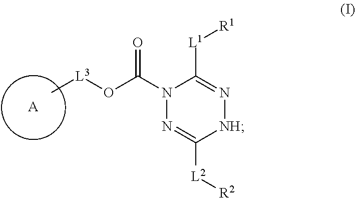

wherein
Ring A is a photolabel moiety;
L¹ is a bond or covalent linker;
L² is a bond or covalent linker;
L³ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X¹ and X² are independently —F, —Cl, —Br, or —I;
n1 and n2 are independently an integer from 0 to 4; and
m1, m2, v1, and v2 are independently 1 or 2.

Embodiment 59. The compound of embodiment 58, wherein L³ is a bond or substituted or unsubstituted C₁-C₄ alkylene.

Embodiment 60. The compound of embodiment 58, wherein L³ is unsubstituted C₁-C₄ alkylene.

Embodiment 61. The compound of embodiment 58, wherein L³ is

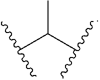 [SMILES: *C(*)C]

Embodiment 62. The compound of embodiment 58, wherein L³ is unsubstituted methylene.

Embodiment 63. The compound of one of embodiments 58 to 62, wherein the photolabel moiety is activated by visible light.

Embodiment 64. The compound of one of embodiments 58 to 62, wherein the photolabel moiety is a substituted or unsubstituted aryl or substituted or unsubstituted heteroaryl.

Embodiment 65. The compound of one of embodiments 58 to 62, wherein the photolabel moiety is

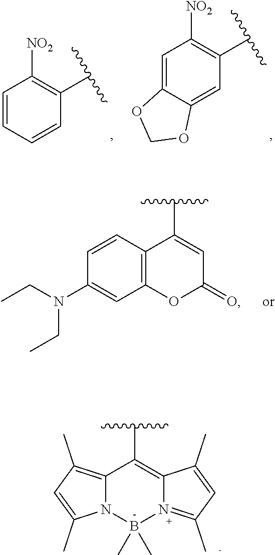

Embodiment 66. The compound of one of embodiments 58 to 65, wherein L¹ is -L¹⁰¹-L¹⁰²-L¹⁰³-L¹⁰⁴-L¹⁰⁵-; and
L¹⁰¹, L¹⁰², L¹⁰³, L¹⁰⁴, and L¹⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

Embodiment 67. The compound of embodiment 66, wherein L¹⁰¹, L¹⁰², L¹⁰³, L¹⁰⁴, and L¹⁰⁵ are a bond.

Embodiment 68. The compound of embodiment 66, wherein
L¹⁰¹ is a substituted or unsubstituted alkylene;
L¹⁰² is a substituted or unsubstituted heteroarylene;
L¹⁰³ is a substituted or unsubstituted alkylene; and
L¹⁰⁴ and L¹⁰⁵ are a bond.

Embodiment 69. The compound of embodiment 66, wherein
L¹⁰¹ is a substituted or unsubstituted alkylene;
L¹⁰² is a substituted or unsubstituted heteroarylene;
L¹⁰³ is a substituted or unsubstituted alkylene; and
L¹⁰⁴ is —C(O)NH—; and
L¹⁰⁵ is a substituted or unsubstituted alkylene.

Embodiment 70. The compound of one of embodiments 58 to 65, wherein L¹ is

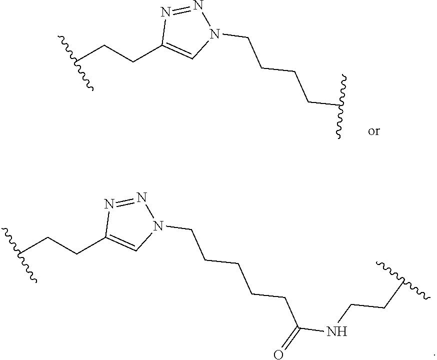 [SMILES: *CCCCn1cc(CC*)nn1 ; *CCNC(=O)CCCCCn1cc(CC*)nn1]

Embodiment 71. The compound of one of embodiments 58 to 70, wherein L² is -L²⁰¹-L²⁰²-L²⁰³-L²⁰⁴-L²⁰⁵-; and
L²⁰¹, L²⁰², L²⁰³, L²⁰⁴, and L²⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

Embodiment 72. The compound of embodiment 71, wherein L²⁰¹, L²⁰², L²⁰³, L²⁰⁴, and L²⁰⁵ are a bond.

Embodiment 73. The compound of one of embodiments 58 to 72, wherein only one of R¹ and R² is a biomolecular moiety.

Embodiment 74. The compound of one of embodiments 58 to 73, wherein R¹ is unsubstituted C₁-C₆ alkyl.

Embodiment 75. The compound of embodiment 74, wherein R¹ is unsubstituted C₂-C₆ alkynyl.

Embodiment 76. The compound of embodiment 75, wherein R¹ is

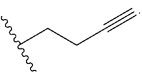 [SMILES: *CCC#C]

Embodiment 77. The compound of one of embodiments 58 to 73, wherein R¹ is unsubstituted phenyl or unsubstituted 5 to 6 membered heteraryl.

Embodiment 78. The compound of one of embodiments 58 to 73, wherein R¹ is unsubstituted phenyl.

Embodiment 79. The compound of one of embodiments 58 to 73, wherein R¹ is unsubstituted pyridyl.

Embodiment 80. The compound of one of embodiments 58 to 73, wherein R¹ is a biomolecular moiety.

Embodiment 81. The compound of embodiment 80, wherein R¹ is a peptide moiety.

Embodiment 82. The compound of embodiment 80, wherein R¹ is a peptide moiety having the sequence LKKGA (SEQ ID NO: 1).

Embodiment 83. The compound of embodiment 80, wherein R¹ is

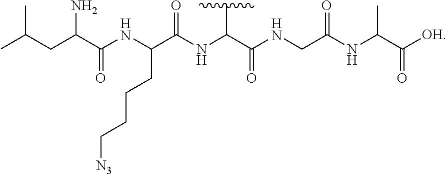 [SMILES: *C(NC(=O)C(CCCCN=[N+]=[N-])NC(=O)C(N)CC(C)C)C(=O)NCC(=O)NC(C)C(=O)O]

Embodiment 84. The compound of embodiment 80, wherein R¹ is —OP(O)(OR^{1C})(OR^{1D}).

Embodiment 85. The compound of embodiment 84, wherein R¹ is

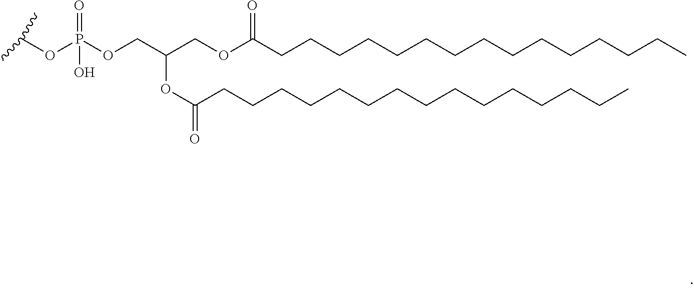 [SMILES: *OP(=O)(O)OCC(COC(=O)CCCCCCCCCCCCCCC)OC(=O)CCCCCCCCCCCCCCC]

Embodiment 86. The compound of one of embodiments 58 to 85, wherein R² is substituted or unsubstituted phenyl or substituted or unsubstituted 5 to 6 membered heteraryl.

Embodiment 87. The compound of embodiment 86, wherein R² is unsubstituted phenyl.

Embodiment 88. The compound of embodiment 86, wherein R² is unsubstituted pyridyl.

Embodiment 89. The compound of embodiment 86, wherein R² is

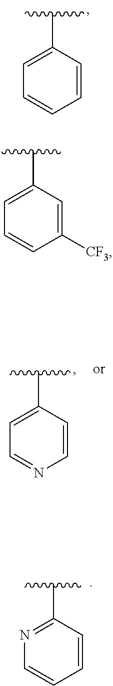

Embodiment 90. The compound of embodiment 58, having the formula:

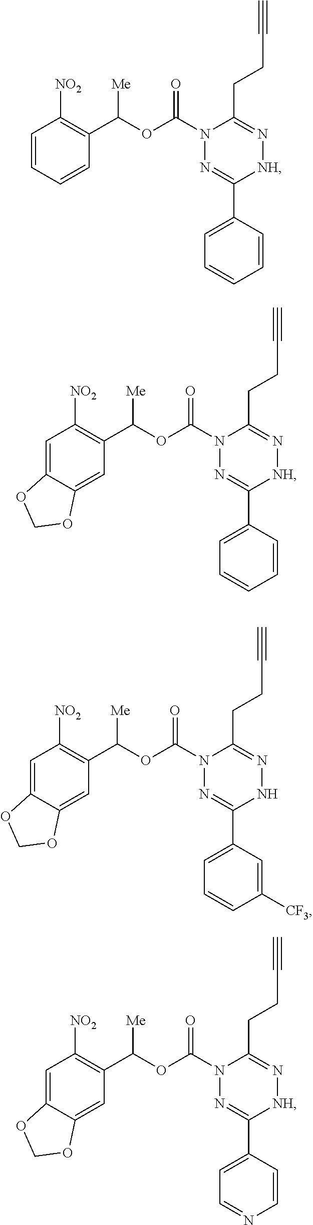

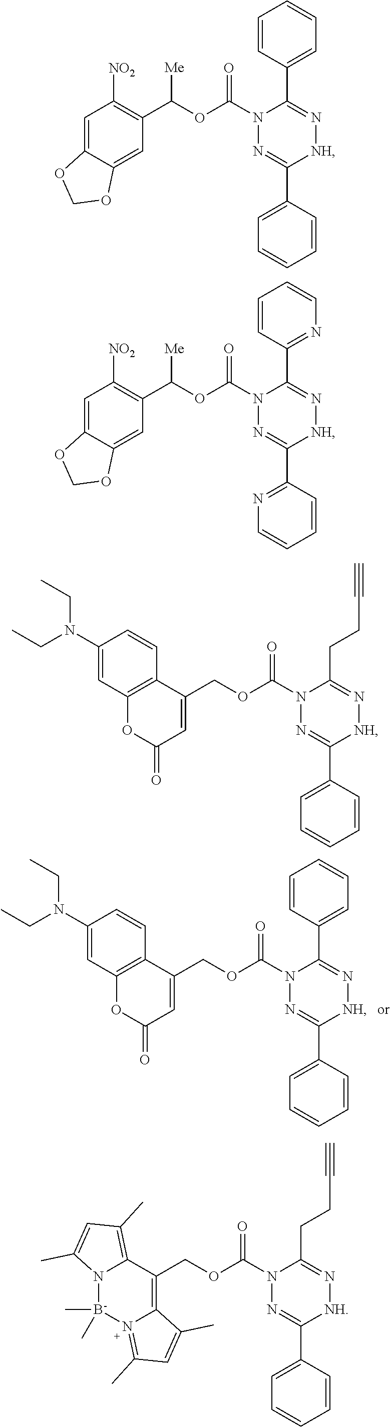

Embodiment 91. The compound of embodiment 58, having the formula:

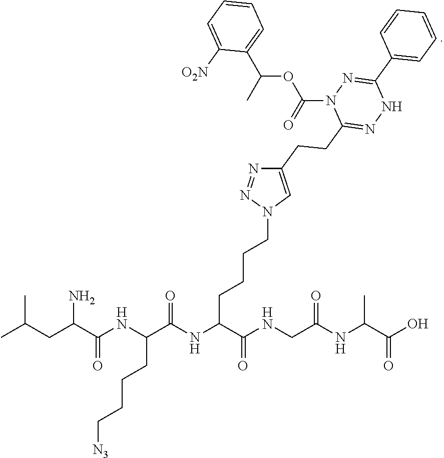 [SMILES: CC(C)CC(N)C(=O)NC(CCCCN=[N+]=[N-])C(=O)NC(CCCCn1cc(CCC2=NNC(c3ccccc3)=NN2C(=O)OC(C)c2ccccc2[N+](=O)[O-])nn1)C(=O)NCC(=O)NC(C)C(=O)O]

Embodiment 92. The compound of embodiment 58, having the formula:

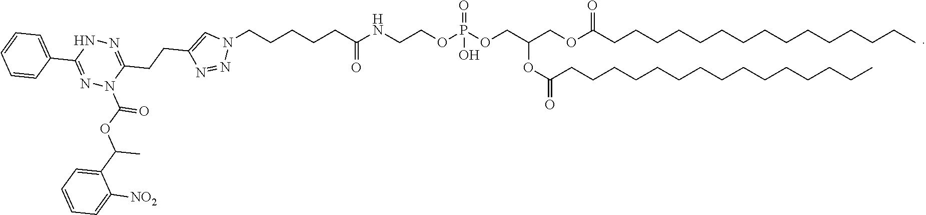 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC]

Embodiment 93. The compound of embodiment 58, having the formula:

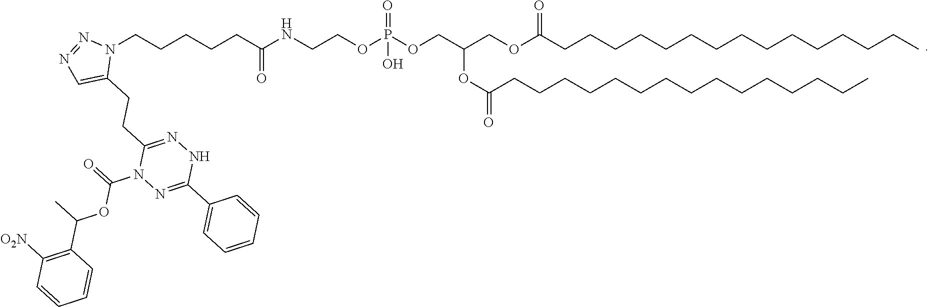 [SMILES: CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC]

Embodiment 94. A pharmaceutical composition comprising a compound of one of embodiments 58 to 93, or a pharmaceutically acceptable salt thereof, and a pharmaceutically acceptable excipient.

Embodiment 95. A method of treating a cancer in a subject in need thereof, said method comprising:
(i) administering to the subject in need thereof a photocaged dihydrotetrazine compound or a compound of one of embodiments 58 to 93, or a pharmaceutically acceptable salt thereof;
(ii) irradiating the photocaged dihydrotetrazine compound or the compound with light to form a tetrazine compound; and
(iii) reacting the tetrazine compound with a dienophile covalently linked to a monovalent form of an anti-cancer agent, thereby releasing the anti-cancer agent.

Embodiment 96. The method of embodiment 95, wherein the cancer is hepatocellular carcinoma.

Embodiment 97. The method of embodiment 95, wherein the cancer is cervical carcinoma.

Embodiment 98. The method of embodiment 95, wherein the cancer is kidney cancer.

Embodiment 99. The method of embodiment 95, wherein the cancer is skin cancer.

Embodiment 100. The method of one of embodiments 95 to 99, wherein the light is visible light.

Embodiment 101. The method of embodiment 100, wherein the light has a wavelength of from about 380 nm to about 700 nm.

Embodiment 102. The method of embodiment 100, wherein the light has a wavelength of from about 400 nm to about 530 nm.

Embodiment 103. The method of embodiment 100, wherein the light has a wavelength of about 405 nm.

Embodiment 104. The method of embodiment 100, wherein the light has a wavelength of about 425 nm.

Embodiment 105. The method of embodiment 100, wherein the light has a wavelength of about 450 nm.

Embodiment 106. The method of embodiment 100, wherein the light has a wavelength of about 525 nm.

Embodiment 107. A method of delivering a drug or a probe in a subject in need thereof, said method comprising:
(i) administering to the subject in need thereof a photocaged dihydrotetrazine compound or a compound of one of embodiments 58 to 93, or a pharmaceutically acceptable salt thereof;
(ii) irradiating the photocaged dihydrotetrazine compound or the compound with light to form a tetrazine compound; and
(iii) reacting the tetrazine compound with a dienophile covalently linked to a monovalent form of the drug or a monovalent form of the probe, thereby releasing the drug or the probe.

Embodiment 108. The method of embodiment 107, wherein the drug is an anti-cancer agent.

Embodiment 109. The method of embodiment 108, wherein the anti-cancer agent is doxorubicin.

Embodiment 110. The method of embodiment 107, wherein the probe is an Alexa Fluor 488 dye.

Embodiment 111. The method of embodiment 107, wherein the probe is an Alexa Fluor 568 dye.

Embodiment 112. The method of one of embodiments 107 to 111, wherein the light is visible light.

Embodiment 113. The method of embodiment 100, wherein the light has a wavelength of from about 380 nm to about 700 nm.

Embodiment 114. The method of embodiment 100, wherein the light has a wavelength of from about 400 nm to about 530 nm.

Embodiment 115. The method of embodiment 100, wherein the light has a wavelength of about 405 nm.

Embodiment 116. The method of embodiment 100, wherein the light has a wavelength of about 425 nm.

Embodiment 117. The method of embodiment 100, wherein the light has a wavelength of about 450 nm.

Embodiment 118. The method of embodiment 100, wherein the light has a wavelength of about 525 nm.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

The use of visible light stimuli to control tetrazine cycloadditions would enable spatiotemporal conjugation, even in the presence of live cells. However past approaches have relied on using ultraviolet light, which is toxic to cells, or achieve light response by using diffusible mediators, which limits spatiotemporal control. Using photocaged dihydrotetrazine, here we demonstrate, inter alia, a visible light-triggered redox activation of tetrazine, which enables spatiotemporal control over bioorthogonal cycloaddition reaction. Photocaged dihydrotetrazines could be quantitively transformed to active tetrazines in aqueous solution after irradiation with visible light (405 nm), leading to rapid cycloaddition reactions with dienophiles such as trans-cyclooctenes (TCO). Using this strategy, we were able to use light to reliably trigger tetrazine cycloadditions in live cells, without toxic side effects. We demonstrate live cell photopatterning with cellular spatial resolution as well as photo-triggered drug release. Given the generality of the approach, we believe light-triggered tetrazine activation will find use in applications where spatial or temporal control over bioconjugation is desired, particularly in the presence of live cells.

Bioorthogonal ligations encompass chemistries that can be used to couple biomolecules in the presence of biologically relevant functional groups (1). The most robust bioorthogonal reactions enable functionalization even in the presence of living systems. Among the numerous bioorthogonal chemistries described to date, tetrazine ligations have enjoyed growing popularity, and have been applied to several bioconjugation applications, including those in the presence of live cells and animals (2). Tetrazine bioorthogonal ligations are attractive for several reasons. The kinetics can be tuned by altering the tetrazine or dienophile, and extremely rapid kinetics (>1000 M⁻¹s⁻¹) are attainable using highly strained trans-cyclooctenes. Imaging applications benefit from the ability of tetrazines to quench fluorophores, making fluorogenic live cell imaging feasible. Finally, recent studies have demonstrated that dienophiles are capable of caging a wide variety of functional groups, which can be released after cycloaddition (3). These so called “click to release” strategies have found increasing application in drug delivery, enzymatic studies, and imaging. While spontaneous tetrazine ligations have already found a wide array of applications, there is growing interest in exploring stimuli responses tetrazine bioorthogonal chemistries (4, 5). These would involve reactants that are normally inert, but ligate in the presence of an appropriate stimuli.

Recent efforts toward engineering light-sensitive dienophiles such as caged cyclopropenes (6) and bicyclononynes (7) have enabled ultraviolet light-induced tetrazine ligations. However, the use of ultraviolet light limits non-toxic use in the presence of live cells, and the caged dienophiles described have relatively modest reaction rates. The fastest reacting and most popular dienophile, trans-cyclooctene, has not yet shown to be amenable to photocaging nor have caged “click to release” dienophiles that are capable of releasing cargo upon cycloaddition. Alternatively, light triggered tetrazine activation would circumvent these issues. Using visible light with a methylene blue photosensitizer, Fox and coworkers developed a light triggered oxidation of air-stable 1,4-dihydrotetrazines to tetrazines (8). However, methylene blue is well-known to be phototoxic to living systems, and the use of a diffusible reagent limits the achievable spatial resolution. Given the drawbacks of previous approaches it is unsurprising that light-responsive tetrazine ligations have been rarely explored in the presence of living cells.

To address these challenges, we pursued a fundamentally straightforward approach to achieve light-triggered tetrazine ligation. It is known that reduction products of tetrazines, dihydrotetrazines, are unreactive to dienophiles and, if sufficiently electron-rich, can spontaneously be oxidized in air to tetrazines. Taking advantage of these properties, we speculated that the secondary amines of dihydrotetrazines might be amenable to modification by visible light cleavable protecting groups. While prior work has not explored such modifications, there is a rich literature of using groups such as nitrophenyl derivatives to cage amines. The photocage would prevent the oxidation to tetrazine, rendering the compound inactive. Once exposed to visible blue light, the cage would be removed, releasing dihydrotetrazine which could spontaneously be air-oxidized to the reactive tetrazine. Subsequently, the tetrazine would be able to react via inverse Diels-Alder cycloaddition with a dienophile of one's choosing. By directly activating the tetrazine, spatial control would be optimized, and the responsiveness to visible light would enable the technique to be used in the presence of living systems.

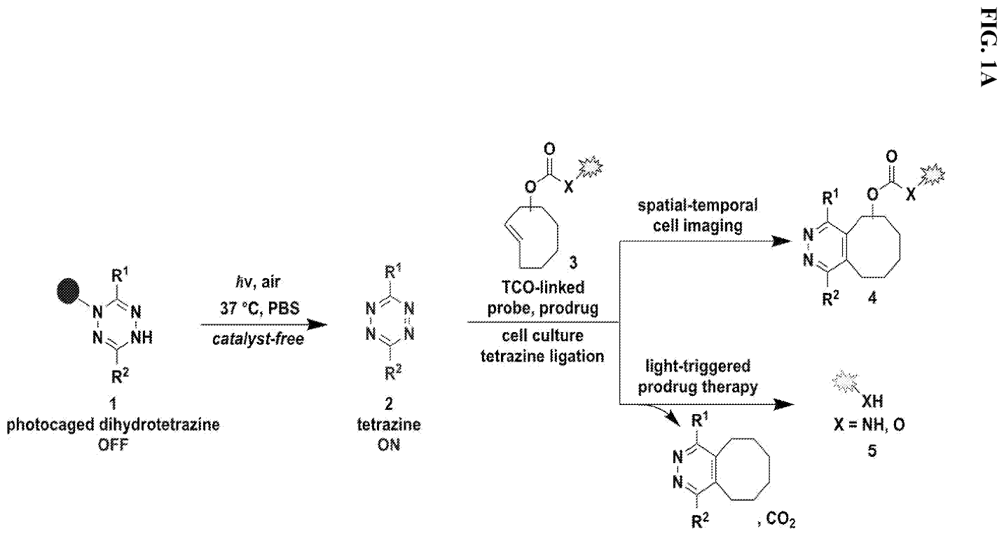

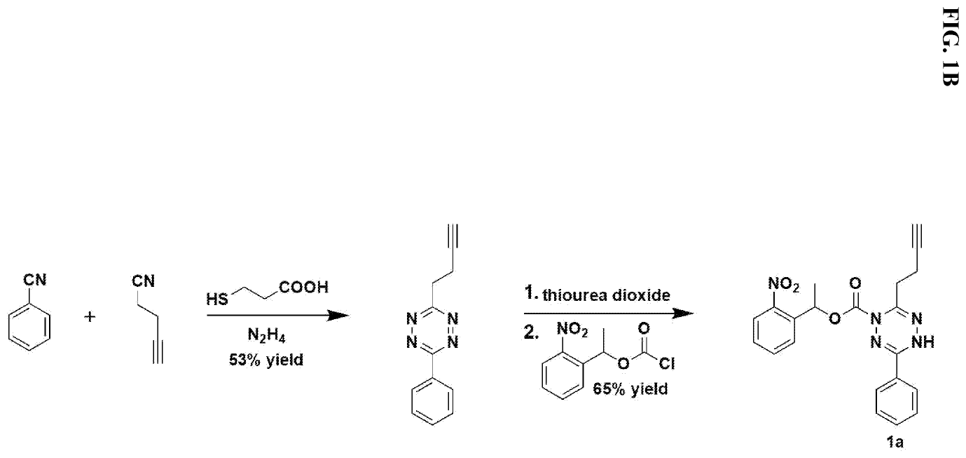

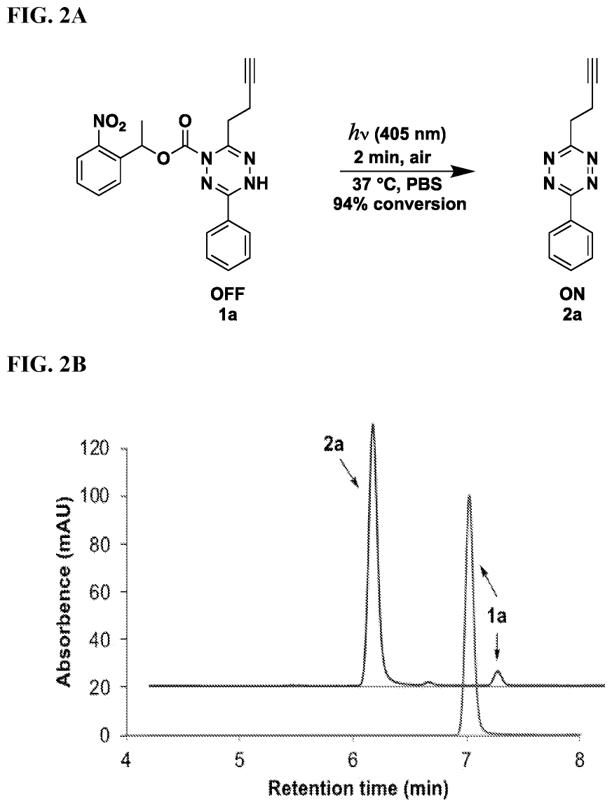

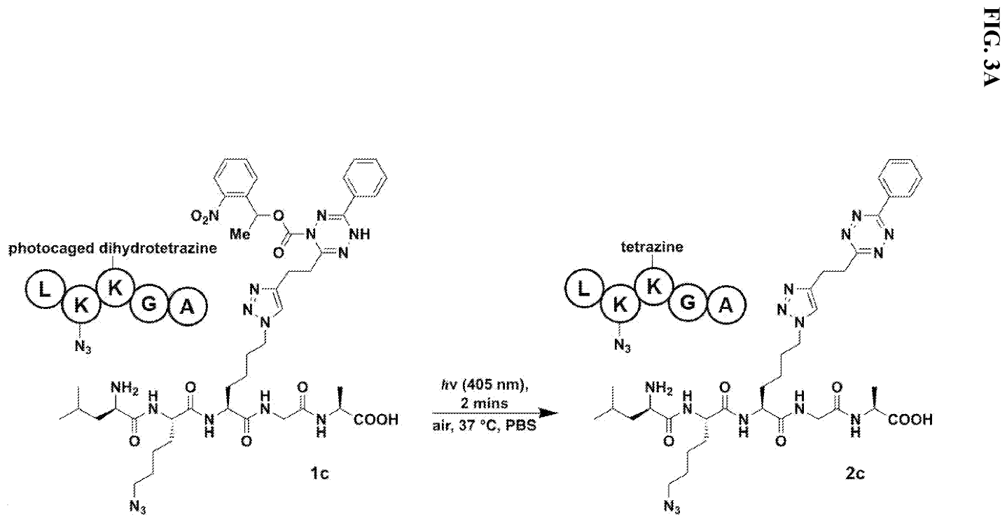

We initiated our studies by synthesizing photocaged dihydrotetrazines 1. Ideally, photocaged dihydrotetrazine 1 should be stable in aqueous solution and the uncaged dihydrotetrazine should rapidly air oxidize to tetrazine 2. With these criteria in mind, we chose 3-(but-3-yn-1-yl)-6-phenyl-1,4-dihydro-1,2,4,5-tetrazine as a model building block, which we reduced to the corresponding dihydrotetrazine. We decided to include a terminal alkyne so that the photocaged dihydrotetrazine could be conjugated to functional molecules by copper-catalyzed azide-alkyne cycloaddition. For the photocleavable protecting group, we decided to utilize 1-(2-nitrophenyl)ethyl carbamate, due to its sensitivity to visible light, and its previous use in cellular studies. By combining the dihydrotetrazine and the photocleavable groups, 1-(2-nitrophenyl)ethyl 6-(but-3-yn-1-yl)-3-phenyl-1,2,4,5-tetrazine-1(4H)-carboxylate photocaged dihydrotetrazine 1a was readily obtained and careful NMR study revealed the confirmed isomer in FIG. 1A. In the absence of irradiation, 1a is stable in aqueous solution at 37° C. with open air for over 4 days. Having photocaged dihydrotetrazines 1 in hand, we next characterized product formation after visible light triggered decaging (FIG. 2A). We found that the 1-(2-nitrophenyl)ethyl carbamate photocage is efficiently cleavable when 1a is irradiated by visible blue light (405 nm, LED) for 2 minutes in PBS (containing 0.1% DMSO) at 37° C. The resulting dihydrotetrazine intermediate is spontaneously oxidized by air, generating the desired 3-(but-3-yn-1-yl)-6-phenyl-1,2,4,5-tetrazine 2a. The HPLC spectra of samples taken from the reaction mixture at different time points revealed that photocaged dihydrotetrazine 1a was almost quantitatively decaged (94% conversion) after the 2 minutes of irradiation (FIG. 2B).

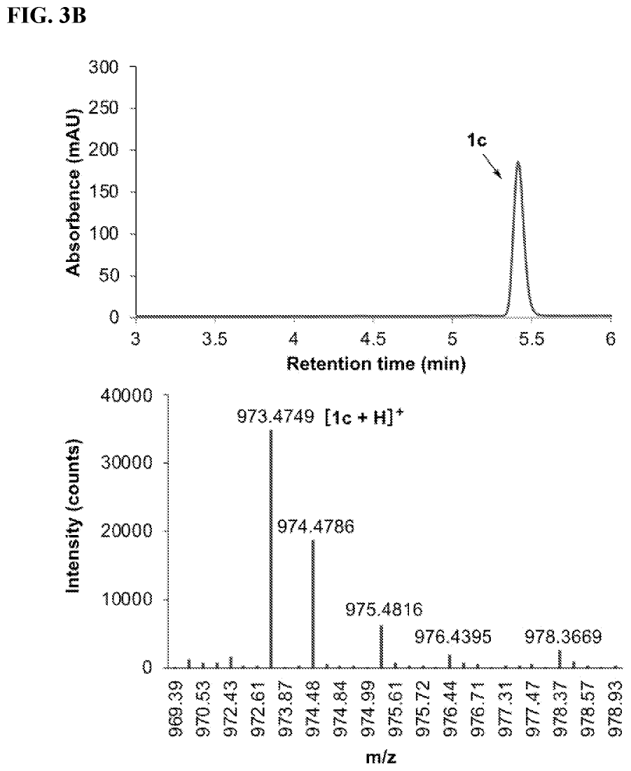

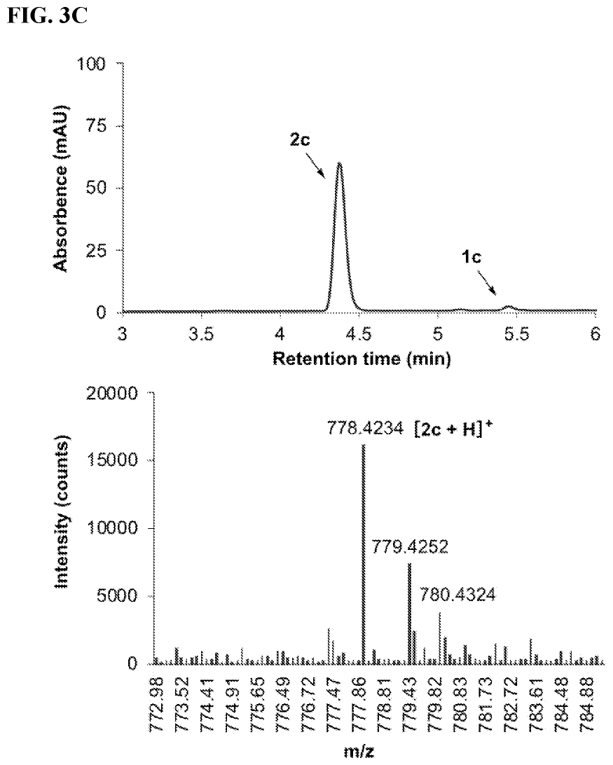

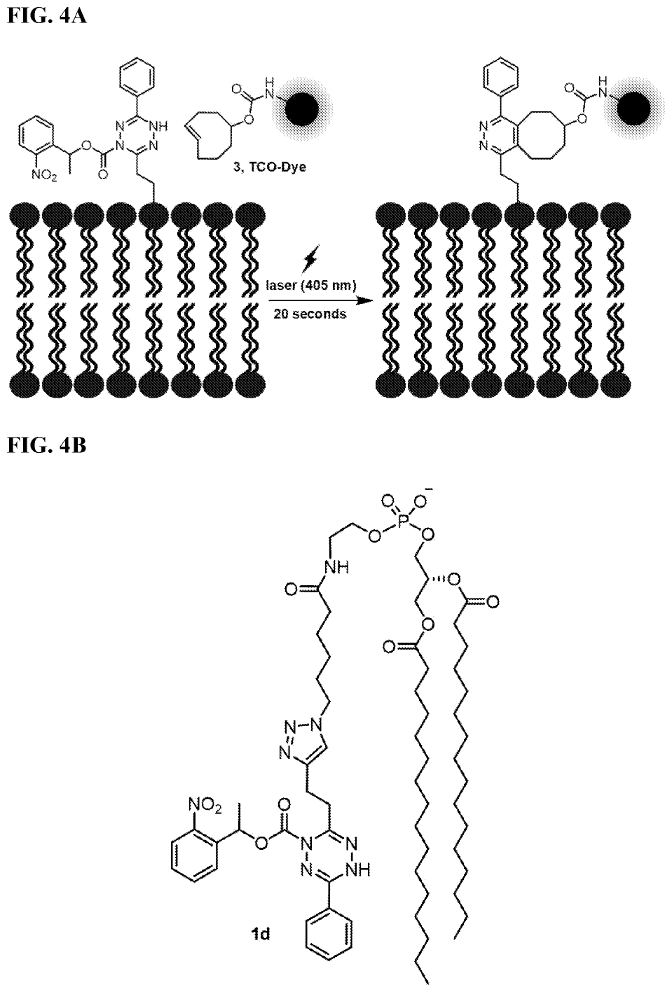

Although tetrazine ligation is a widely used bioorthogonal tool, tetrazine-containing amino acid has been rarely explored in the solid-phase peptide synthesis (SPPS) due to dramatic degradations of tetrazines under the protection conditions, such as 4-methylpiperidine/DMF solution. Markedly, photocaged dihydrotetrazine 1a proves to be stable in the presence of 4-methylpiperidine/DMF solution, so that we were able to employ photocaged dihydrotetrazine-containing amino acid in SPPS and obtained the desired tetrazine-containing peptide in the late stage. Here we have synthesized unnatural amino acid photocaged dihydrotetrazine-lysine 1b (12 mg, 15 μmol) and obtained a five-amino-acid peptide 1c through practical SPPS in 49% yield (FIG. 3A). After irradiation of dihydrotetrazine-peptide 1c (10 μM) for 2 minutes in PBS at 37° C., tetrazine-peptide 2c could been readily achieved, which contains two common bioorthogonal groups, including an azido group and a tetrazine group.

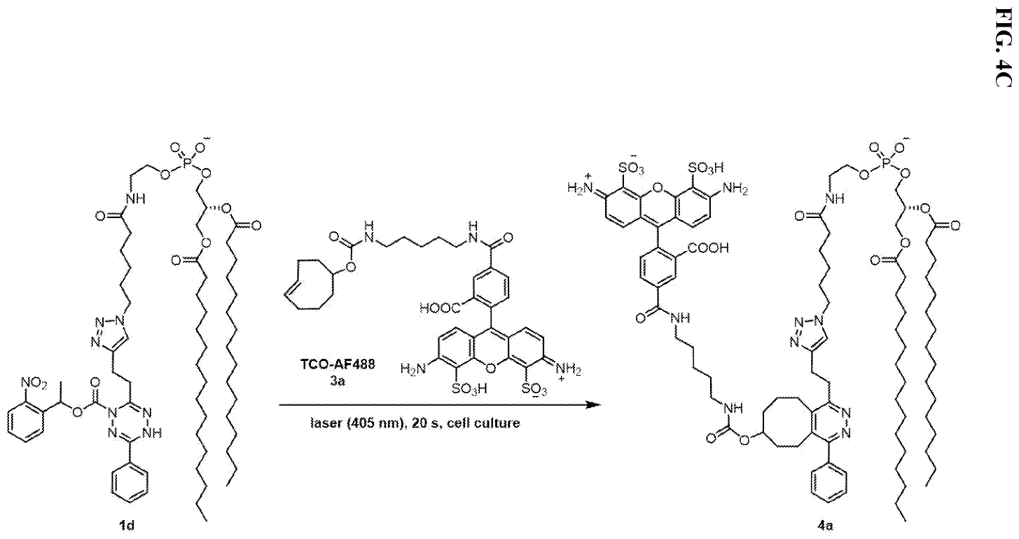

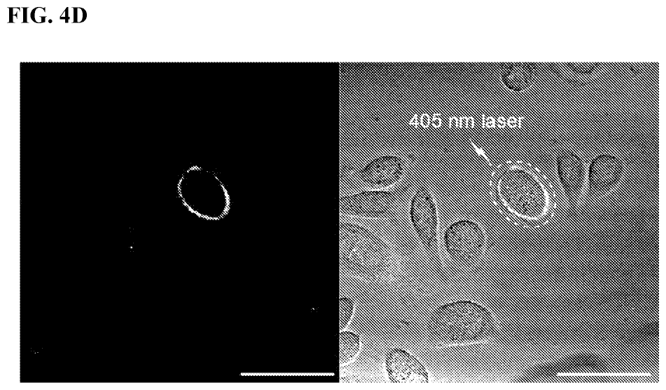

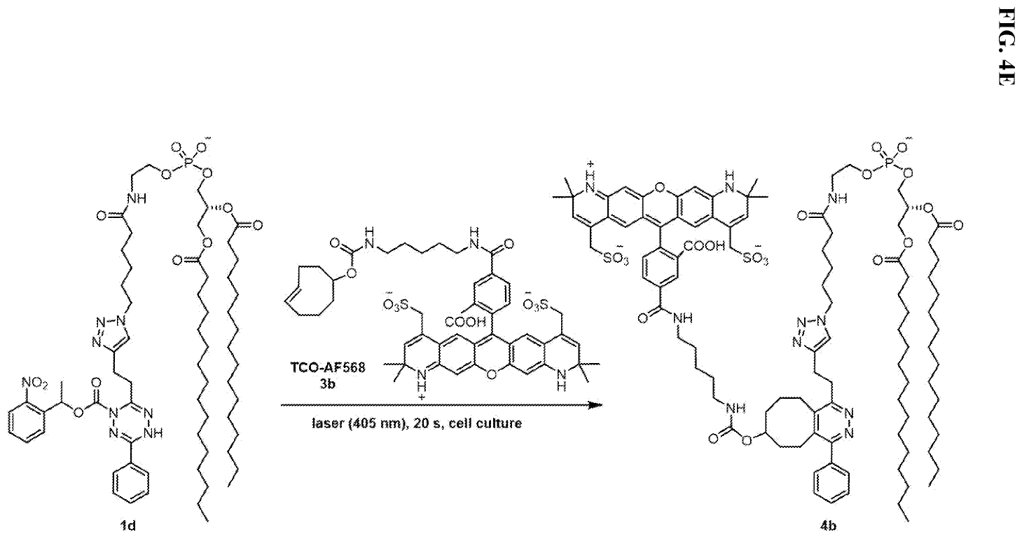

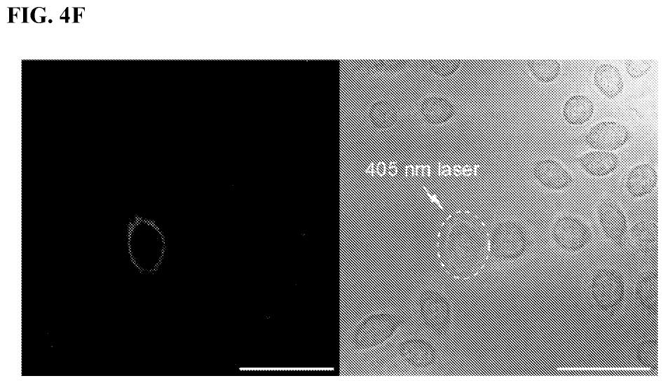

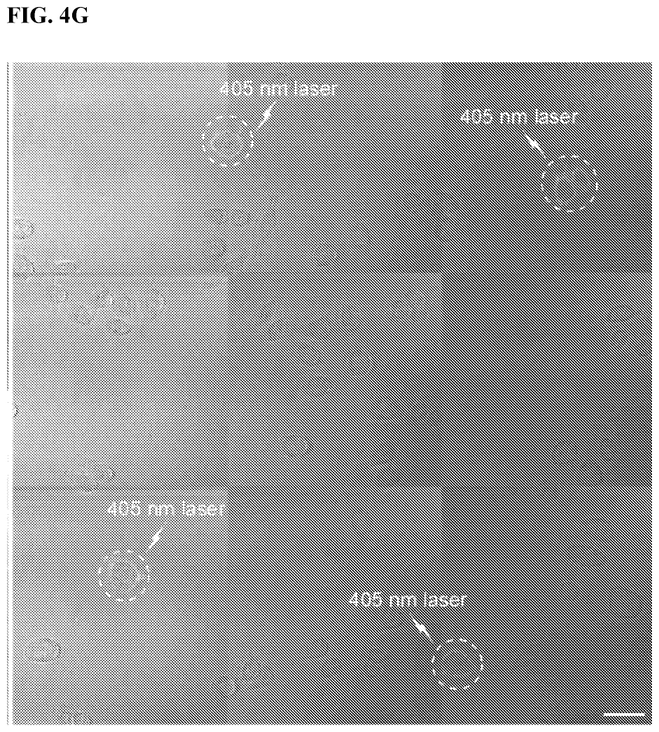

As discussed, the use of photocaged dihydrotetrazines that are responsive to visible light have two major advantages compared to approaches that rely on soluble photosensitizers. First, the uncaging is compatible with live cells since cages that are responsive to visible light can be installed, which avoids phototoxicity. In contrast, sensitizers like methylene blue are known to be highly phototoxic. Second, the spatial resolution of tetrazine activation is limited when using a diffusible photosensitizer for activation. The sensitizer has the ability to diffuse away from where light stimulation takes place and activate tetrazines not intended for reaction. In contrast, only photocaged dihydrotetrazines directly stimulated by the light source will be activated for ligation. With this in mind, we decided to explore whether are methodology could be utilized for light-controlled spatial patterning of live cells. To introduce photocaged dihydrotetrazines to cells, we labeled an azido-containing diacylphospholipid with 1a to synthesize photocaged dihydrotetrazine-diacylphospholipid 1d. This artificial phospholipid readily incorporated in live cell membranes. Upon irradiation with visible light (405 nm LED) 1d rapidly reacted with a water-soluble trans-cyclooctene modified Alexa Fluor 488 dye (TCO-AF488) 3a forming cycloaddition product 4a, as determined by LCMS. We therefore utilized fluorophore 3a for light triggered patterning of live cell membranes that had incorporated photocaged dihydrotetrazine-diacylphospholipid 1d. We utilized a 405 nm laser to spatially restrict tetrazine activation to specific cell populations, and confocal microscopy to visualize formation of the fluorescent product. Initially, HelaS3 cancer cells was incubated in 200 μL photocaged dihydrotetrazine-diacylphospholipid 1d (80 nM) in PBS (containing 0.1% DMSO) at 37° C. for 5 minutes. Then 200 μL of TCO-AF488 3a (10 nM) or TCO-AF568 3b (10 nM) in PBS (containing 0.1% DMSO) was added onto cells, and irradiated targeted cells by the laser (405 nm) of confocal microscopy for 20 seconds. After incubation at 37° C. for 5 minutes, cell media was exchanged with 200 μL DMEM complete media then use confocal microscopy for live cell imaging. Confocal fluorescence microscopy revealed specific fluorescence patterning of single cellular membrane under the excitation region (FIG. 4D). Using this turn on tetrazine ligation, we are also able to image the single cell membrane of living HelaS3 cancer cells by Alexa Fluor 568 dye (FIG. 4F).

An exciting emerging application of tetrazine ligations are the so called “click to release” strategies, which typically involve utilizing a dienophile to cage a functional group. Upon cycloaddition with tetrazine, tautomerization occurs leading the elimination of the functional group and release. In this way, the dienophile can be thought of as a protecting group, masking functional molecules until the tetrazine trigger occurs. Initial studies focused on the elimination of amines from trans-cyclooctenes, and applied the strategy to the release of doxorubicin, a common anti-cancer compound. Other applications include the controlling the delivery of drugs in vivo and the unmasking of active site function groups to control enzyme activity, which is accomplished by site-specific incorporation of masked lysine into the active site of enzymes. More recently, the technique has been extended to the release of alcohols and carboxylic acids. We speculated that combining light activated tetrazine formation with “click to release” strategies would be a powerful way to gain control over functional group release in living systems, in particular the controlled release of therapeutics.

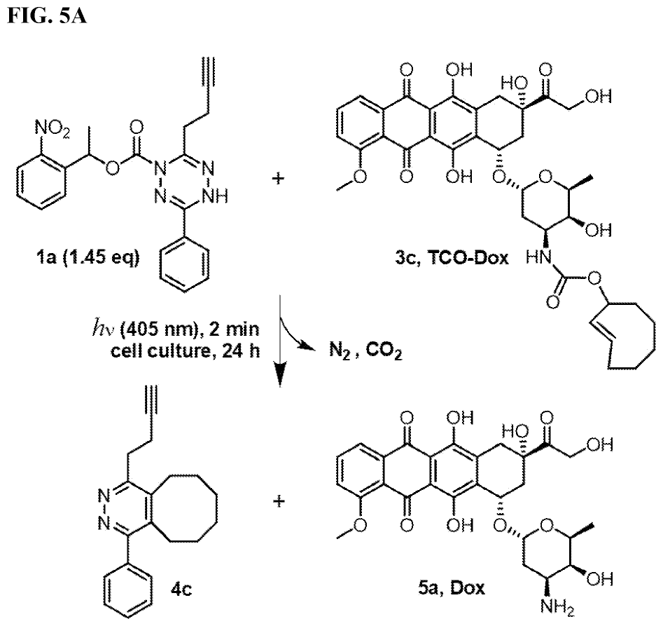

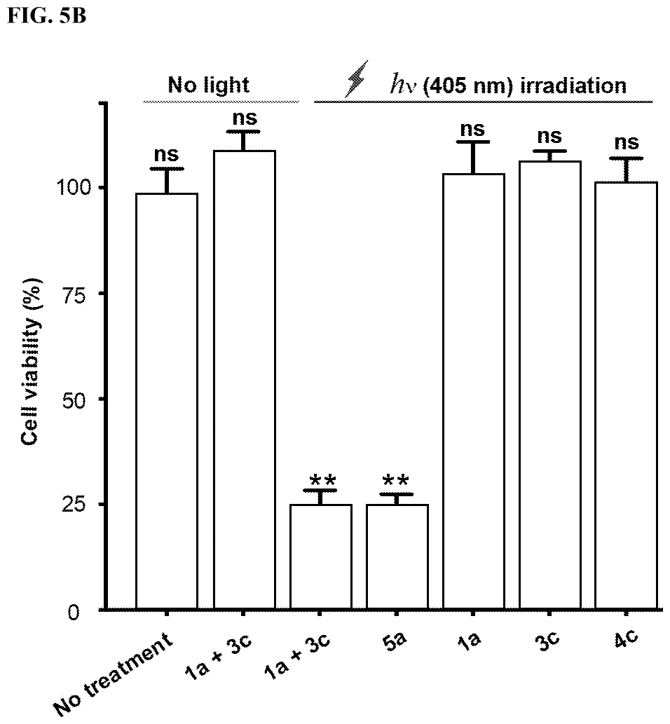

We therefore next explored coupling of light-triggered tetrazine ligation with click to release, to enable the light activated drug delivery. Tetrazine 2a is generated in situ after a light-activation of photocaged dihydrotetrazine 1a. After irradiation, tetrazine 2a can under cycloaddition with inactive dienophile-caged prodrugs, resulting in release of the active drug. As a prodrug, we synthesized trans-cyclooctene carbamate caged doxorubicin 3c (TCO-Dox), which liberates doxorubicin 6a (Dox) after undergoing cycloaddition with tetrazines. Doxorubicin is a well-known anti-cancer drug, and therefore we speculated that we would be able to use light to stimulate delivery to cancer cells through a dual activation process (FIG. 5A), resulting in apoptosis. Initially we tested the dual activation process in vitro. We irradiated a reaction mixture consisting of photocaged dihydrotetrazine 1a (8 μM) and TCO-Dox 3c (5.5 μM) by LED light (405 nm) for 2 minutes in PBS (containing 0.1% DMSO) at 37° C. This was followed by incubation at 37° C. for 24 hours. Samples from the reaction mixture were taken at different time points, and analysis by HPLC-MS showed that TCO-Dox 3c was fully consumed after 24 hours. In the absence of irradiation, the mixture of photocaged dihydrotetrazine 1a (8 μM) and TCO-Dox 3b (5.5 μM) showed no reaction.

Based on the positive dual activation results obtained from the in vitro experiments, we next tested if light-triggered tetrazine ligation enabled prodrug therapy could be carried out in the presence of live cancer cells (FIG. 5B). When the cancer cells were treated with a mixture of photocaged dihydrotetrazine 1a (8 μM) and TCO-Dox 3c (5.5 μM), no significant influence on cell viability was observed compared to control experiments with no treatment. However, upon irradiation by LED light (405 nm) for 2 minutes, followed by 24 incubation, significant cell death was observed. Loss of cell viability was similar to that observed when cells were directly treated with Dox 6a (5.5 μM) under the same conditions. Notably, there was no influence on cell viability when cells were treated with either photocaged dihydrotetrazine 1a (8 μM) or TCO-Dox 3c (5.5 μM) alone. Furthermore, no influence on cell viability was observed when the cells were treated by an external stimulus visible blue light (405 nm) for 2 minutes. These results demonstrate that photocaged dihydrotetrazine can be utilized for the light-triggered release of bioactive compounds, such as doxorubicin, in the presence of living cells.

We imagine future applications would enable optical control of drug release, perhaps a during image guided surgery or as a variant of photodynamic therapies. Combined with strategies that use dienophiles to cage enzyme function, the light-controlled formation of tetrazine would add spatiotemporal control over biomolecular activation. Additionally, the ability to use dienophiles to cage a broad range of functional groups, such as amines, alcohols, and carboxylic acids, offers a complementary strategy to achieve visible light-controlled release of functional groups in a general and modular fashion.

Here, we demonstrate, inter alia, that a light-triggered redox activation of dihydrotetrazine/tetrazine enabled bioorthogonal tetrazine ligations with a high spatiotemporal precision. We have applied this turn-on bioorthogonal ligation in prodrug therapy study and successfully achieved a light-controlled delivery of chemotherapy drug doxorubicin. Moreover, we were also able to manipulate the cell membrane with tetrazine-linked diacylphospholipids at designated space and time, which allows for spatial-temporal live cell imaging. We believe this novel tool holds significant promise in targeting drug delivery, biomolecular engineering, materials science, and beyond.

### REFERENCES FOR EXAMPLE 1

1. Bertozzi, C. R. A decade of bioorthogonal chemistry. Acc. Chem. Res. 44, 651-653 (2011). 2. Oliveira, M. B. L., Guo, Z., & Bernardes, G. J. L. Inverse electron demand Diels-Alder reactions in chemical biology. Chem. Soc. Rev. 46, 4895-4950 (2017). 3. Carlson, J. C. T., Mikula, H., & Weissleder, R. Unraveling tetrazine-triggered bioorthogonal elimination enables chemical tools for ultrafast release and universal cleavage. J. Am. Chem. Soc. 140, 3603-3612 (2018). 4. Tasdelen, M. A., & Yagci, Y. Light-induced click reactions. Angew. Chem. Int. Ed. 52, 5930-5938 (2013). 5. Mueller, J. O., Schmidt, F. G., Blinco, J. P., & Bamer-Kowollik C. Visible-light-induced click chemistry. Angew. Chem. Int. Ed. 54, 10284-10288 (2015). 6. Kumar, P., Jiang, T., Li, S., Zainul, O., & Laughlin, S. T. Caged cyclopropenes for controlling bioorthogonal reactivity. Org. Biomol. Chem. 16, 4081-4085 (2018). 7. Mayer, S. V., Murnauer, A., von Wrisberg, M., Jokisch, M., & Lang, K. Photo-induced and rapid labeling of tetrazine-bearing proteins via cyclopropenone-caged bicyclononynes. Angew. Chem. Int. Ed. 58, 15876-15882 (2019). 8. Zhang, H., Trout, W. S., Liu, S., Andrade, G. A., Hudson, D. A., Scinto, S. L., Dicker, K. T., Li, Y., Lazouski, N., Rosenthal, J., Thorpe, C., Jia, X., & Fox, J. M. Rapid bioorthogonal chemistry turn-on through enzymatic or long wavelength photocatalytic activation of tetrazine ligation. J Am. Chem. Soc. 138, 5978-5983 (2016).

### Example 2: Light-Activated Tetrazines Enable Precision Live-Cell Bioorthogonal Chemistry

Bioorthogonal cycloaddition reactions between tetrazines and strained dienophiles are widely used in protein, lipid, and glycan labeling due to their extremely rapid kinetics. While applications of tetrazine ligations are growing in academia and industry, it has so far been challenging to control this chemistry to achieve the high degrees of spatial and temporal precision necessary for modifying mammalian cells with single-cell resolution. Here we demonstrate, inter alia, visible light-activated formation of tetrazines from photocaged dihydrotetrazines, which enables live-cell spatiotemporal control of rapid biorthogonal cycloaddition reactions between tetrazines and dienophiles such as trans-cyclooctenes (TCOs). Photocaged dihydrotetrazines are stable in conditions that normally degrade tetrazines, enabling efficient early-stage incorporation of bioorthogonal handles into biomolecules such as peptides. Photocaged dihydrotetrazines allow the use of non-toxic visible light to trigger tetrazine ligations on living mammalian cells. By tagging reactive phospholipids with fluorophores, we demonstrate modification of HeLa cell membranes with single-cell spatial resolution. Finally, we show that photo-triggered therapy is possible by coupling tetrazine photoactivation with strategies that uncage prodrugs in response to tetrazine ligation, opening up new methods for photopharmacology and precision drug delivery using bioorthogonal chemistry.

Bioorthogonal ligations encompass coupling reactions that have considerable utility in living systems (1-3). Among the numerous bioorthogonal reactions described to date, rapid inverse electron demand Diels-Alder reactions between tetrazines and dienophiles have found widespread use in chemical biology and material science, since their introduction in 2008 (4-7). For example, tetrazine ligations have been used in whole animal proteome labeling (8), the capture of circulating tumor cells (9), tracking lipid modifications (10), and imaging glycosylation (11). In addition, dienophiles can cage a variety of functional groups, which are released after the cycloaddition reaction with Tetrazine (12, 13) and such “click to release” strategies have been exploited for tumor imaging (14), controlling enzyme activity (15), and drug delivery (16). Recently, tetrazine ligation-triggered drug delivery has entered human phase I clinical trials (17, 18). As applications expand, there is a growing need for methods that can precisely control the reaction in the presence of living cells. Due to its noninvasive nature, and the high degree of spatial and temporal resolution attainable, light has become the tool of choice for remote manipulation of biological systems. For instance, photoactivatable green-fluorescent protein (GFP) has enabled numerous studies that track protein movement or mark specific cells in a population (19). Similarly, light controllable tetrazine ligations would open up applications such as cell surface engineering with single-cell precision and timed drug release (20). Recent efforts toward engineering light-sensitive dienophiles such as caged cyclopropenes (21) and bicyclononynes (22) have enabled ultraviolet light-induced tetrazine ligations with relatively modest reaction rates. However, ultraviolet light is toxic to living cells, particularly mammalian cells, which limits applications (23). Trans-cyclooctenes (TCOs), the fastest reacting dienophiles, and caged “click to release” dienophiles have not yet been shown to be amenable to photocaging (24). In principle, light-triggered tetrazine formation would circumvent these issues. A visible light-triggered oxidation of air-stable 1,4-dihydrotetrazines to tetrazines using methylene blue as a photosensitizer has been developed (25). However, methylene blue is toxic and the use of a diffusible mediator limits spatiotemporal control (26). We hypothesized that direct activation of tetrazines using visible light would address these challenges and enable precision chemistry in biological systems.

To develop a light-triggered tetrazine ligation with high spatiotemporal precision, we explored whether a tetrazine precursor could be caged by a photocleavable protecting group (FIG. 1A). Dihydrotetrazine, a precursor for tetrazine, is unreactive to dienophiles (25) and, if sufficiently electron-rich, is spontaneously oxidized by air through reaction with oxygen (24). Therefore, we asked whether the secondary amines of dihydrotetrazine could be modified with visible light-cleavable protecting groups, such as nitrophenyl derivatives (27). Photocaging would prevent the oxidation of dihydrotetrazine to tetrazine, creating a compound that is inactive to cycloaddition with strained dienophiles. The caging group would be removed upon exposure to visible light, leading to an in situ formation of tetrazine. Subsequently, the tetrazine would be able to react rapidly with a dienophile through inverse Diels-Alder cycloaddition. By directly activating the tetrazine, spatial control could be achieved, and the responsiveness to visible light would enable live-cell applications.

A photocaged dihydrotetrazine should be stable in aqueous solution and the dihydrotetrazine should rapidly be oxidized to tetrazine by air. With this hypothesis in mind, we synthesized photocaged dihydrotetrazine 1a from 3-(but-3-yn-1-yl)-6-phenyl-1,2,4,5-tetrazine, which could be converted to the corresponding dihydrotetrazine using the reductant thiourea dioxide (FIG. 1B). 1-(2-Nitrophenyl)ethyl carbamate was investigated as the photocleavable functional group, due to its sensitivity to visible blue light (28) and biocompatibility (29). After reacting the dihydrotetrazine intermediate with the selected photocleavable group, the desired product 1-(2-nitrophenyl)ethyl 6-(but-3-yn-1-yl)-3-phenyl-1,2,4,5-tetrazine-1(4H)-carboxylate photocaged dihydrotetrazine 1a was obtained. Nuclear magnetic resonance (NMR) studies confirmed that the secondary amine adjacent to the alkyl group of dihydrotetrazine 1a is functionalized. We analyzed light-triggered tetrazine formation from photocaged dihydrotetrazine 1a (FIGS. 2A-2B). HPLC revealed that 1a was nearly quantitatively decaged (94% conversion) when irradiated by visible blue light (405 nm, LED, 18 W) for 2 minutes in aqueous buffer (PBS containing 0.1% DMSO) at 37° C. and the desired tetrazine 2a was in situ formed. After demonstrating visible blue light-activated formation of tetrazine 2a, we verified the stability of photocaged dihydrotetrazine 1a in aqueous solution in the absence of irradiation. In phosphate-buffered saline (PBS) or cell lysate at 37° C., degradation of 1a was not observed over 1 day, and less than 1% of decomposition occurred after 4 days of incubation. A major advantage of tetrazine ligations is the high rate of reaction with ring-strained trans-cyclooctene dienophiles. We measured a second-order rate constant of 101±3 M⁻¹ s⁻¹ between 2a and a model strained dienophile, trans-4-cycloocten-1-ol (TCO-OH), by monitoring the disappearance of the characteristic tetrazine visible absorption at 521 nm under pseudo first-order conditions (13).

We next explored the scope of photocaged dihydrotetrazine and have successfully incorporated various photoprotecting groups with 3-(but-3-yn-1-yl)-6-phenyl-1,4-dihydro-1,2,4,5-tetrazine (FIG. 6 and FIG. 7). Meanwhile we have investigated different substituted dihydrotetrazines. We were able to achieve 6-nitropiperonyl methyl photoprotecting electron poor 3,6-diphenyl-1,4-dihydro-1,2,4,5-tetrazine and 3,6-di(pyridin-2-yl)-1,4-dihydro-1,2,4,5-tetrazine, which could afford corresponding 3,6-diphenyl-1,2,4,5-tetrazine and 3,6-di(pyridin-2-yl)-1,2,4,5-tetrazine upon exposure to blue light.

Having synthesized various photocaged dihydrotetrazines and demonstrated in situ formation of tetrazines upon exposure to corresponding visible light, we next moved forward with exploring bioconjugation applications. Tetrazines are susceptible to hydrolysis, particularly in the presence of nucleophiles and base (30, 31). This feature has hampered applications of tetrazine ligations, particularly if tetrazines are required to be stable in physiological media for lengthy periods of time before cycloaddition, such as for pretargeted imaging, drug delivery applications, or in cases where early-stage modification of tetrazine is desired. For instance, although tetrazine ligations have been widely used to modify peptides, the tetrazine is typically introduced in the late-stage of peptide synthesis due to its incompatibility with solid-phase peptide synthesis (SPPS) reaction conditions (32). Electron poor tetrazine-containing amino acids are not used in Fmoc solid-phase peptide synthesis (SPPS) due to their degradation during the standard repeated Fmoc deprotection conditions (e.g., 4-methylpiperidine/DMF).

Considering the stability of photocaged dihydrotetrazines, we asked if such groups could act as photoprotection moieties, masking latent tetrazines through harsh conditions that would normally be degradative. For example, in 4-methylpiperidine/DMF solution, 40% of tetrazine 2a degrades in 30 minutes. In contrast, photocaged dihydrotetrazine 1a is stable in 4-methylpiperidine/DMF solution with no detectable degradation observed under the same reaction conditions. Therefore, we hypothesized that photocaged dihydrotetrazines would be tolerated during SPPS. To test this, we synthesized an unnatural Fmoc protected amino acid containing photocaged dihydrotetrazine, 1b. We employed 1b in SPPS to obtain a five-amino-acid peptide 1c in 50% yield. After LED irradiation of photocaged dihydrotetrazine-peptide 1c (10 μM) for 2 minutes in PBS at 37° C., tetrazine-peptide 2c was formed in 96% yield (FIGS. 3A-3C). Being able to directly use photoprotected tetrazine amino acids facilitates SPPS of peptides that contain multiple bioorthogonal handles, such as an azido group and tetrazine, making it easier to site-specifically label peptides with multiple probes.

Since our strategy utilizes visible light to directly generate tetrazines from their precursors, we asked whether photocaged dihydrotetrazines could spatiotemporally modify living cells, for instance by covalently labeling membrane lipids (FIG. 4A). To remodel cell membranes with photocaged dihydrotetrazines, we appended 1a to a derivative of 1,2-dipalmitoyl-sn-glycero-3-phosphoethanolamine (DPPE) to form photocaged dihydrotetrazine-diacylphospholipid 1d (FIG. 4B). Upon irradiation with visible light (405 nm, LED, 18 W), 1d reacted rapidly with a water-soluble trans-cyclooctene modified Alexa Fluor 488 dye (TCO-AF488) 3a forming cycloaddition product 4a (FIG. 4C), as determined by LC-MS. To incorporate photocaged dihydrotetrazine onto cell membranes, adherent HeLa S3 cells were incubated with 60 nM photocaged dihydrotetrazine-diacylphospholipid 1d in PBS solution (containing 0.1% DMSO) at 37° C. for 5 minutes. Excess 1d was removed by washing cells with PBS solution. The washed cells were then incubated with 3 nM TCO-AF488 3a in PBS solution (containing 0.1% DMSO). To trigger in situ formation of tetrazine and the subsequent bioorthogonal tetrazine ligation, a single cell among a population of cells was selectively irradiated with a 405 nm laser (20 mW) for 20 seconds using a ZEISS 880 laser scanning microscope. Five minutes after the laser uncaging event, unreacted TCO-AF488 was removed by exchanging the solution with fresh cell culture medium. Fluorescence live-cell imaging was performed to reveal if tetrazine ligation had taken place on the membrane of the laser irradiated cell. Fluorescence labeling by AF488 was only observed on the membrane of the laser irradiated cell and not on adjacent cells, illustrating that precise spatiotemporal photoactivation of tetrazine ligation can be achieved using photocaged dihydrotetrazine-diacylphospholipid 1d (FIG. 4D).

To demonstrate the versatility of the light-activated single-cell manipulation, we also performed labeling with an alternative trans-cyclooctene modified dye, Alexa Fluor 568 (TCO-AF568) 3b (FIG. 4E). Additionally, our technique can be applied to other types of mammalian cells, for example, Hep 3B human liver cancer cells. Finally, to test the robustness of spatial photoactivation, populations of 1-2 cells located at four different locations inside a 0.75 mm by 0.75 mm area were selectively laser irradiated at 405 nm to trigger tetrazine ligation, modifying the associated cell membranes (FIG. 4G). Fluorescence labeling was only observed on the laser irradiated cells, demonstrating that reliable spatiotemporal labeling of living cells can be achieved by photoactivation of surface tetrazines.

An application of tetrazine ligation is the so called “click to release” strategy, which typically involves utilizing a dienophile to cage a bioactive molecule such as a drug (24). Upon reaction with tetrazine, tautomerization of the cycloadduct occurs leading to elimination and drug release (12). We hypothesized that combining light-activated tetrazine formation with “click to release” strategies would facilitate the controlled release of therapeutics in the presence of living systems for photopharmacology. To couple light-activated tetrazine ligation with “click to release”, we synthesized a dienophile modified prodrug, trans-cyclooctene carbamate-caged doxorubicin 3c (TCO-Dox), which liberates doxorubicin 5a (Dox) after undergoing cycloaddition reaction with tetrazine. Doxorubicin is an anticancer drug, and we therefore sought to use light to stimulate doxorubicin delivery to cancer cells through a dual activation process (FIG. 5A), triggering apoptosis. We first tested the dual activation process by irradiating a reaction mixture consisting of photocaged dihydrotetrazine 1a (8 μM) and TCO-Dox 3c (5.5 μM) with LED light (405 nm, 18 W) for 2 minutes in PBS solution (containing 0.1% DMSO) at 37° C. This was followed by incubation at 37° C. for 24 hours. Samples from the reaction mixture were taken at different time points, and analysis by HPLC-MS showed that 91% of TCO-Dox 3c was converted to doxorubicin 5a after 24 hours. In the absence of irradiation, the mixture of photocaged dihydrotetrazine 1a (8 μM) and TCO-Dox 3c (5.5 μM) showed no reaction.

Based on the positive results obtained, we tested whether light-triggered drug delivery could be carried out in the presence of living cancer cells (FIG. 5B). When Hep 3B human liver cancer cells were treated with a mixture of photocaged dihydrotetrazine 1a (8 μM), and TCO-Dox 3c (5.5 μM), no decrease in cell viability was observed compared to untreated cells. However, upon irradiation with LED light (405 nm, 18W) for 2 minutes, followed by incubation for 24 hours, a 75.1±3% reduction of cell viability was observed. The loss of cell viability was similar to that observed when cells were directly treated with Dox 5a (5.5 μM) under the same conditions. There was no influence on cell viability when cells were irradiated with LED light for 2 minutes in the presence of either photocaged dihydrotetrazine 1a (8 μM), TCO-Dox 3c (5.5 μM), or side product 4c (5.5 μM) alone. These results demonstrate that photocaged dihydrotetrazines can be utilized for the light-triggered release of bioactive compounds, such as chemotherapeutics, in the presence of living cells.

We have demonstrated a methodology for the photoactivation of tetrazines that enables biomolecular labeling, spatiotemporal modification of live-cell membranes with single-cell precision, and photopharmacology when combined with “click to release” strategies. Tetrazine instability is a well-recognized obstacle to their use, and we have found that photocaged tetrazine precursors are highly stable, even in the presence of strong bases which rapidly degrade tetrazines. Given the stability of photocaged dihydrotetrazines, we expect they will find broad application as a general tetrazine protecting group. Photocaged dihydrotetrazines would be especially useful in conditions known to degrade tetrazines, such as those encountered during the installation of ¹⁸F radionuclides for PET imaging (30), or for live-cell pulse-chase experiments where tetrazine reactivity would be required to be maintained for an arbitrary amount of time before reaction (33). Indeed, preliminary results indicate that photocaged dihydrotetrazines, unlike tetrazines, are very stable under the conditions typically used for fluorination. Since our method directly activates tetrazine precursors, high spatiotemporal precision is achievable. By modifying phospholipids on cell surfaces, we showed that single-cell activation is feasible. The technique could enable monitoring of lipid trafficking and dynamics in living cells by controlling where and when caged tetrazines on lipids are activated and following their transport by post-labeling with dienophile modified fluorophores (34). Light-activated release of the chemotherapeutic doxorubicin was carried out by combining photoactivation of tetrazine formation with “click to release” strategies. The photocaged tetrazine precursor and light alone showed negligible toxicity, demonstrating the biocompatibility of the technique. Such optically controlled drug release may have practical application in image guided surgery and photodynamic therapy (35). Future studies will explore alternative caging groups that activate in response to longer wavelengths of light, enabling multiplexing and facilitating in vivo studies (27). The basic concept we present might also be extended to other amine caging functionalities capable of masking dihydrotetrazines, allowing rapid biorthogonal ligation in response to additional stimuli such as enzymatic activity, pH, or the presence of metal complexes (36, 37).

### REFERENCES FOR EXAMPLE 2

1. Saxon, E. & Bertozzi, C. R. Science. 287, 2007-2010 (2000). 2. Prescher, J. A., Dube, D. H. & Bertozzi, C. R. Nature. 430, 873-877 (2004). 3. Devaraj, N. K. ACS Cent. Sci. 4, 952-959 (2018). 4. Blackman, M. L., Royzen, M. & Fox, J. M. J Am. Chem. Soc. 130, 13518-13519 (2008). 5. Devaraj, N. K., Weissleder, R. & Hilderbrand, S. A. Bioconjugate Chem. 19, 2297-2299 (2008). 6. Oliveira, B. L., Guo, Z. & Bernardes, G. J. L. Chem. Soc. Rev. 46, 4895-4950 (2017). 7. Nguyen, S. S. & Prescher, J. A. Nat. Rev. Chem. 4, 476-489 (2020). 8. Elliott, T. S., Townsley, F. M., Bianco, A., Ernst, R. J., Sachdeva, A., Elsasser, S. J., Davis, L., Lang, K., Pisa, R., Greiss, S., Lilley, K. S. & Chin, J. W. Nat. Biotechnol. 32, 465-472 (2014). 9. Dong, J., Jan, Y. J., Cheng, J., Zhang, R. Y., Meng, M., Smalley, M., Chen, P. J., Tang, X., Tseng, P., Bao, L., Huang, T. Y., Zhou, D., Liu, Y., Chai, X., Zhang, H., Zhou, A., Agopian, V. G., Posadas, E. M., Shyue, J. J., Jonas, S. J., Weiss, P. S., Li, M., Zheng, G., Yu, H. H., Zhao, M., Tseng, H. R. & Zhu, Y. Sci. Adv. 5, eaav9186 (2019). 10. Liang, D., Wu, K., Tei, R., Bumpus, T. W., Ye, J. & Baskin, J. M. Proc. Natl. Acad. Sci. USA 116, 15453-15462 (2019). 11. Agarwal, P., Beahm, B. J., Shieh, P. & Bertozzi, C. R. Angew. Chem. Int. Ed. 54, 11504-11510 (2015). 12. Carlson, J. C. T., Mikula, H. & Weissleder, R. J Am. Chem. Soc. 140, 3603-3612 (2018). 13. Versteegen, R. M., ten Hoeve, W., Rossin, R., de Geus, M. A. R., Janssen, H. M. & Robillard, M. S. Angew. Chem. Int. Ed. 57, 10494-10499 (2018). 14. Li, H., Conde, J., Guerreiro, A. & Bernardes, G. J. L. Angew. Chem. Int. Ed. 59, 16023-16032 (2020). 15. Li, J., Jia, S. & Chen, P. R. Nat. Chem. Biol. 10, 1003-1005 (2014). 16. Ji, X., Pan, Z., Yu, B., De La Cruz, L. K., Zheng, Y., Ke, B. & Wang, B. Chem. Soc. Rev. 48, 1077-1094 (2019). 17. Czuban, M., Srinivasan, S., Yee, N. A., Agustin, E., Koliszak, A., Miller, E., Khan, I., Quinones, I., Noory, H., Motola, C., Volkmer, R., Di Luca, M., Trampuz, A., Royzen, M. & Mejia Oneto, J. M. ACS Cent. Sci. 4, 1624-1632 (2018). 18. Wu, K., Yee, N., Srinivasan, S., Mahmoodi, A., Zakharian, M., Mejia Oneto, J. M. & Royzen, M. Preprint at http://doi.org/10.26434/chemrxiv.13087715.v1 (2020). 19. Patterson, G. H. & Lippincott-Schwartz, J. A photoactivatable GFP for selective photolabeling of proteins and cells. Science. 297, 1873-1887 (2002). 20. Kumar, G. S. & Lin, Q. Chem. Rev. http://doi.org/10.1021/acs.chemrev.0c00799 (2020). 21. Kumar, P., Jiang, T., Li, S., Zainul, O. & Laughlin, S. T. Org. Biomol. Chem. 16, 4081-4085 (2018). 22. Mayer, S. V., Mumauer, A., von Wrisberg, M. K., Jokisch, M. L. & Lang, K. Angew. Chem. Int. Ed. 58, 15876-15882 (2019). 23. Hockberger, P. E. Photochem. Photobiol. 76, 561-579 (2002). 24. Versteegen, R. M., Rossin, R., ten Hoeve, W., Janssen, H. M. & Robillard, M. S. Angew. Chem. Int. Ed. 52, 14112-14116 (2013). 25. Zhang, H., Trout, W. S., Liu, S., Andrade, G. A., Hudson, D. A., Scinto, S. L., Dicker, K. T., Li, Y., Lazouski, N., Rosenthal, J., Thorpe, C., Jia, X. & Fox, J. M. J Am. Chem. Soc. 138, 5978-5983 (2016). 26. da Costa S. R., da Costa Monteiro M., da Silva Junior F. M. R. & Sandrini J. Z. Cell Biol. Int. 40, 895-905 (2016). 27. Hansen, M. J., Velema, W. A., Lerch, M. M., Szymanski, W. & Feringa, B. L. Chem. Soc. Rev. 44, 3358-3377 (2015). 28. Corrie, J. E. T., Barth, A., Munasinghe, V. R. N., Trentham, D. R. & Hutter, M. C. J Am. Chem. Soc. 125, 8546-8554 (2003). 29. Zhao, Y. R., Zheng, Q., Dakin, K., Xu, K., Martinez, M. L. & Li, W. H. J. Am. Chem. Soc. 126, 4653-4663 (2004). 30. Li, Z., Cai, H., Hassink, M., Blackman, M. L., Brown, R. C. D., Conti, P. S. & Fox, J. M. Chem. Commun. 46, 8043-8045 (2010). 31. Selvaraj, R., Liu, S., Hassink, M., Huang, C. W., Yap, L. P., Park, R., Fox, J. M., Li, Z. & Conti, P. S. Bioorg. Med. Chem. Lett. 21, 5011-5014 (2011). 32. Pagel, M. J. Pept. Sci. 25, e3141. (2019). 33. Willis, J. C. W. & Chin, J. W. Nat. Chem. 10, 831-837 (2018). 34. Tamura, T., Fujisawa, A., Tsuchiya, M., Shen, Y., Nagao, K., Kawano, S., Tamura, Y., Endo, T., Umeda, M. & Hamachi, I. Nat. Chem. Biol. 16, 1361-1367 (2020). 35. Kwiatkowski, S., Knap, B., Przystupski, D., Saczko, J., Kedzierska, E., Knap-Czop, K., Kotlinska, J., Michel, O., Kotowski, K. & Kulbacka, J. Biomed. Pharmacother. 106, 1098-1107 (2013). 36. Cao, Z., Li, W., Liu, R., Li, X., Li, H., Liu, L., Chen, Y., Lv, C. & Liu, Y. Biomed. Pharmacother. 118, 109340 (2019). 37. Li, J., Yu, J., Zhao, J., Wang, J., Zheng, S., Lin, S., Chen, L., Yang, M., Jia, S., Zhang, X. & Chen, P. R. Nat. Chem. 6, 352-361 (2014).

### Example 3: Experimental Procedures and Characterization Data

### Synthesis of Asymmetric Dihydrotetrazine

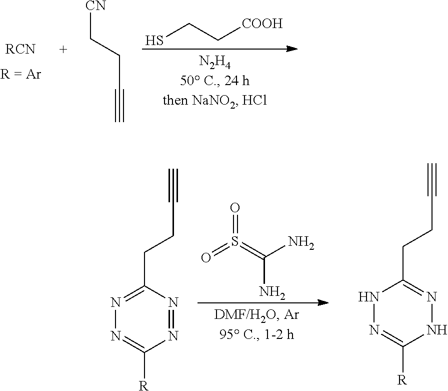

A mixture of aromatic nitrile (6 mmol), 4-pentynenitrile (24 mmol), and 3-mercaptopropionic acid (264 μL, 3 mmol) was cooled to 0° C. under argon. Anhydrous hydrazine (4.6 mL, 96 mmol) was added dropwise to the mixture. The reaction mixture was stirred in an oil bath at 50° C. for 24 hours. Upon completion, the reaction solution was cooled with ice water. A solution of sodium nitrite in ice water was slowly added into the reaction mixture, followed by slow addition of 1 M HCl. Addition of 1 M HCl continued until gas evolution ceased. Then, the reaction mixture was extracted with CH₂Cl₂ and washed with a saturated solution of NaCl in water. The extract was combined, dried over Na₂SO₄, and concentrated under reduced pressure. The residue was purified by column chromatography on silica gel using 30%-50% CH₂Cl₂/Hexane as the eluents yielding the compound tetrazine as a pink solid.

In a sealed flask, the reductant thiourea dioxide (1.5 mmol, 1.5 eq) was added to a solution of tetrazine (1.0 mmol) in 7.5 mL of DMF/H₂O (v/v=10/1) at room temperature under argon. The reaction mixture was stirred in an oil bath at 95° C. for 1-2 hours. Upon completion, the color of the reaction mixture changed from pink to light yellow. Under argon, 20 mL of EtOAc was added the reaction mixture, which was washed by 3 mL of H₂O. The extract was combined and concentrated by reduced pressure. The residue was purified by column chromatography on silica gel under argon using CH₂Cl₂ to 4% MeOH/CH₂Cl₂ as the eluents yielding the desired dihydrotetrazine.

### Synthesis of Photocaged Dihydrotetrazine

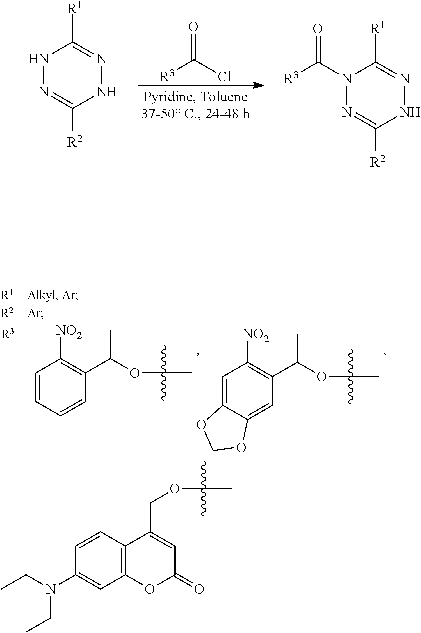

Under argon, anhydrous pyridine was added to dihydrotetrazine (1.0 eq) in a sealed flask, followed by slow addition of a solution of photocaged carbonochloridate in toluene (2.0 eq) at 0° C. The reaction mixture was stirred at 37-50° C. for 24-48 h. Upon completion, the reaction mixture was concentrated under reduced pressure. The residue was purified by column chromatography on silica gel using 50%-100% CH₂Cl₂/Hexane to 4% MeOH/CH₂Cl₂ as the eluents yielding the desired photocaged dihydrotetrazine.

### Synthesis of BODIPY Photocaged Dihydrotetrazine

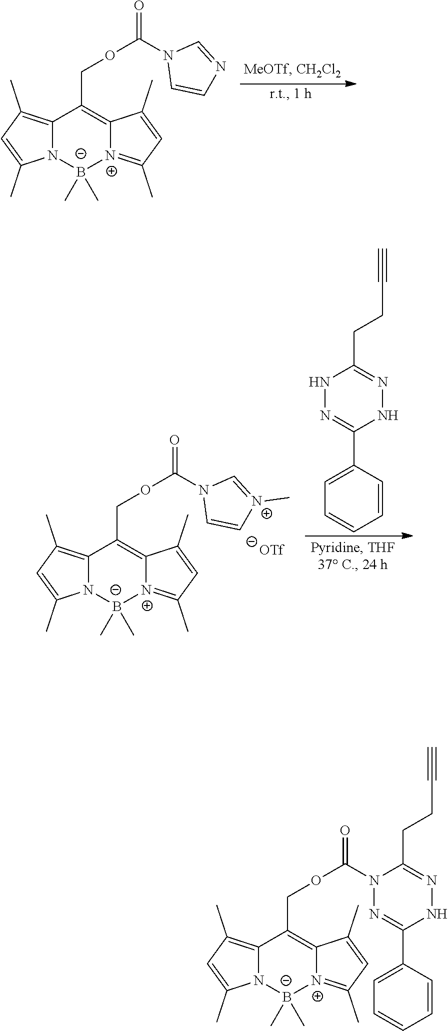

Under argon, anhydrous CH₂Cl₂ was added to imidazole-protected BODIPY alcohol (1.0 eq) in a sealed flask, followed by slow addition of methyl trifluoromethanesulfonate (1.1 eq) at 0° C. The reaction mixture was stirred at room temperature for 1 h, then the solvent was removed by reduced pressure and high vacuum. Under argon, slowly add a solution of dihydrotetrazine (0.5 eq) in anhydrous pyridine/THF (v/v=1/4) to the reaction mixture at 0° C. Then the reaction was stirred at 37° C. for 48 h.

Upon completion, the reaction mixture was concentrated under reduced pressure. The residue was purified by high performance liquid chromatography with ZORBAX SB-C18 column with Phase A/Phase B gradients [Phase A: MeOH with 0.1% formic acid, Phase B: H₂O with 0.1% formic acid]. 50%-70% Phase A in Phase B, 1 minute, and 70%-80% Phase A in Phase B, 4 minutes, 80%-90% Phase A in Phase B, 5 minutes, 90%-100% Phase A in Phase B, 3 minutes, then 100% Phase A, 9 minutes.

### 3-(but-3-yn-1-yl)-6-phenyl-1,2,4,5-tetrazine

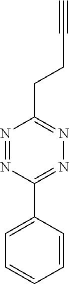 [SMILES: C#CCCc1nnc(-c2ccccc2)nn1]

¹H NMR (400 MHz, (CD₃)₂SO): δ 8.49 (dd, J=8.1, 1.7 Hz, 2H), 7.74-7.64 (m, 3H), 3.55-3.46 (m, 2H), 2.91-2.83 (m, 3H). ¹³C NMR (101 MHz, (CD₃)₂SO): δ 168.15, 163.72, 132.71, 131.75, 129.57, 127.57, 82.85, 72.53, 33.28, 16.12. HRMS m/z (ESI): calcd. for C₁₂H₁₃N₄ [M+H]⁺: 213.1135; found: 213.1133.

### 3-(but-3-yn-1-yl)-6-(3-(trifluoromethyl)phenyl)-1,2,4,5-tetrazine

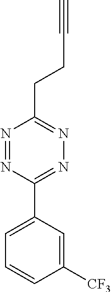 [SMILES: C#CCCc1nnc(-c2cccc(C(F)(F)F)c2)nn1]

¹H NMR (400 MHz, (CD₃)₂SO): δ 8.76 (dt, J=7.9, 1.5 Hz, 1H), 8.73-8.67 (m, 1H), 8.12-8.03 (m, 1H), 7.93 (t, J=7.9 Hz, 1H), 3.54 (t, J=7.3 Hz, 2H), 2.92-2.83 (m, 3H). ¹³C NMR (101 MHz, (CD₃)₂SO): δ 168.54, 162.81, 132.98, 131.46, 130.91, 130.66, 130.35, 130.03, 129.71, 129.12, 129.08, 129.04, 128.99, 127.93, 125.23, 123.83, 123.78, 123.74, 123.70, 122.52, 119.80, 82.73, 72.52, 33.34, 16.10. HRMS m/z (ESI): calcd. for C₁₃H₁₀ F₃N₄ [M+H]⁺: 279.0852; found: 279.0851.

### 3-(but-3-yn-1-yl)-6-(pyridin-4-yl)-1,2,4,5-tetrazine

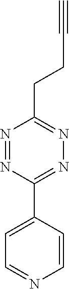 [SMILES: C#CCCc1nnc(-c2ccncc2)nn1]

¹H NMR (800 MHz, (CD₃)₂SO): δ 8.96-8.88 (m, 2H), 8.43-8.35 (m, 2H), 3.55 (t, J=7.2 Hz, 2H), 2.9-2.85 (m, 3H). ¹³C NMR (201 MHz, (CD₃)₂SO): δ 168.96, 162.75, 151.09, 139.25, 121.14, 82.72, 72.51, 33.41, 16.01. HRMS m/z (ESI): calcd. for C₁₁H₁₀N₅ [M+H]⁺: 212.0931; found: 212.0932.

### 3-(but-3-yn-1-yl)-6-phenyl-1,4-dihydro-1,2,4,5-tetrazine

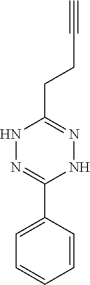 [SMILES: C#CCCC1=NNC(c2ccccc2)=NN1]

¹H NMR (600 MHz, (CD₃)₂SO): δ 8.64 (s, 1H), 8.34 (s, 1H), 7.77-7.72 (m, 2H), 7.47-7.37 (m, 3H), 2.81 (d, J=2.3 Hz, 1H), 2.43 (td, J=7.5, 2.7 Hz, 2H), 2.30 (t, J=7.5 Hz, 2H). ¹³C NMR (201 MHz, (CD₃)₂SO): δ 149.23, 147.22, 130.48, 129.90, 128.41, 125.76, 83.47, 71.66, 28.91, 14.68. HRMS m/z (ESI): calcd. for C₁₂H₁₃N₄ [M+H]⁺: 213.1135; found: 213.1133.

### 3-(but-3-yn-1-yl)-6-(3-(trifluoromethyl)phenyl)-1,4-dihydro-1,2,4,5-tetrazine

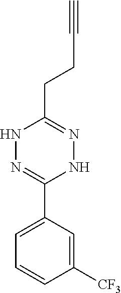 [SMILES: C#CCCC1=NNC(c2cccc(C(F)(F)F)c2)=NN1]

¹H NMR (800 MHz, (CD₃)₂SO): δ 8.86 (s, 1H), 8.49 (s, 1H), 8.09 (s, 1H), 8.05 (d, J=7.9 Hz, 1H), 7.80 (d, J=7.8 Hz, 1H), 7.66 (t, J=7.9 Hz, 1H), 2.82 (d, J=2.3, 1H), 2.43 (td, J=7.5, 2.6 Hz, 2H), 2.31 (t, J=7.5 Hz, 2H). ¹³C NMR (201 MHz, (CD₃)₂SO): δ 149.33, 146.09, 131.48, 129.73, 129.69, 129.59, 129.43, 129.27, 129.11, 126.47, 126.45, 124.66, 123.31, 122.27, 122.25, 122.23, 122.21, 83.41, 71.72, 28.84, 14.69. HRMS m/z (ESI): calcd. for C₁₃H₁₂ F₃N₄ [M+H]⁺: 281.1009; found: 281.1009.

### 3-(but-3-yn-1-yl)-6-(pyridin-4-yl)-1,4-dihydro-1,2,4,5-tetrazine

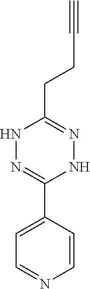 [SMILES: C#CCCC1=NNC(c2ccncc2)=NN1]

¹H NMR (800 MHz, (CD₃)₂SO): δ 8.80 (s, 1H), 8.63 (dd, J=5.9, 1.1 Hz, 2H), 8.59 (s, 1H), 7.69 (dt, J=4.5, 1.1 Hz, 2H), 2.82 (m, 1H), 2.43 (ddd, J=8.1, 7.0, 2.7 Hz, 2H), 2.30 (t, J=7.5 Hz, 2H). ¹³C NMR (201 MHz, (CD₃)₂SO): δ 150.06, 149.09, 145.39, 137.66, 119.72, 83.35, 71.72, 28.77, 14.62. HRMS m/z (ESI): calcd. for C₁₁H₁₂N₅ [M+H]⁺: 214.1087; found: 214.1087.

General procedure to prepare 1-(2-nitrophenyl)ethyl 6-(but-3-yn-1-yl)-3-phenyl-1,2,4,5-tetrazine-1(4H)-carboxylate 1a: In a sealed flask, the reductant thiourea dioxide (160 mg, 1.5 mmol) was added to a solution of tetrazine 2a (210 mg, 1.0 mmol) in 7.5 mL of dimethylformamide (DMF)/H₂O (v/v=10/1) at room temperature under argon. The reaction mixture was stirred in an oil bath at 95° C. for 1 hour. Upon completion, the color of the reaction mixture changed from pink to light yellow. The reaction solvent was removed under reduced pressure and the residue was dried under high vacuum overnight, resulting in a powder containing dihydrotetrazine, which was transferred to a sealed flask under argon and directly used for the next step. Under argon, 15 mL of anhydrous pyridine was added, followed by slow addition of a solution of 1-(2-nitrophenyl)ethyl carbonochloridate (532 mg, 2.3 mmol) in toluene (2.5 mL) at room temperature. The reaction mixture was stirred at 95° C. for 24 hours. Upon completion, the reaction mixture was concentrated under reduced pressure. The residue was purified by column chromatography on silica gel using 50%-100% CH₂Cl₂/Hexane to 15% MeOH/CH₂Cl₂ as the eluents, yielding the title compound 1a as a pale-yellow solid (263 mg, 65%).

### 1-(2-nitrophenyl)ethyl 6-(but-3-yn-1-yl)-3-phenyl-1,2,4,5-tetrazine-1(4H)-carboxylate

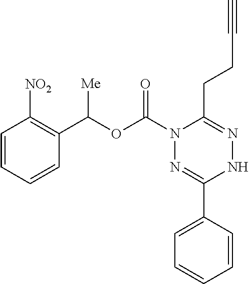 [SMILES: C#CCCC1=NNC(c2ccccc2)=NN1C(=O)OC(C)c1ccccc1[N+](=O)[O-]]

¹H NMR (800 MHz, (CD₃)₂SO): δ 10.28 (s, 1H), 8.01 (dd, J=8.2, 1.2 Hz, 1H), 7.88-7.84 (m, 2H), 7.84-7.80 (m, 1H), 7.78 (dd, J=8.0, 1.5 Hz, 1H), 7.62-7.56 (m, 2H), 7.52 (t, J=7.6 Hz, 2H), 6.22 (q, J=6.5 Hz, 1H), 2.82 (t, J=7.2 Hz, 2H), 2.79 (t, J=2.6 Hz, 1H), 2.37 (tt, J=7.3, 2.5 Hz, 2H), 1.69 (d, J=6.5 Hz, 3H). ¹³C NMR (201 MHz, (CD₃)₂SO): δ 155.68, 149.32, 147.46, 142.03, 136.76, 134.21, 131.69, 129.16, 128.79, 128.40, 127.35, 126.94, 124.32, 82.87, 71.94, 69.51, 29.72, 21.61, 15.27. HRMS m/z (ESI): calcd. for C₂₁H₂₀N₅O₄ [M+H]⁺: 406.1510; found: 406.1507.

### 1-(6-nitrobenzo[d][1,3]dioxol-5-yl)ethyl 6-(but-3-yn-1-yl)-3-phenyl-1,2,4,5-tetrazine-1(4H)-carboxylate

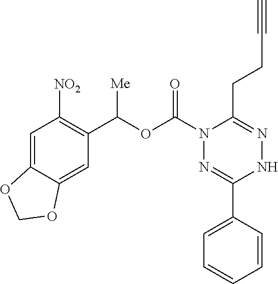 [SMILES: C#CCCC1=NNC(c2ccccc2)=NN1C(=O)OC(C)c1cc2c(cc1[N+](=O)[O-])OCO2]

¹H NMR (800 MHz, (CD₃)₂SO): δ 10.29 (s, 1H), 7.87 (d, J=7.1 Hz, 2H), 7.62 (s, 1H), 7.61-7.57 (m, 1H), 7.55-7.49 (m, 2H), 7.23 (s, 1H), 6.24 (m, 3H), 2.83 (t, J=7.3 Hz, 2H), 2.79 (t, J=2.6 Hz, 1H), 2.39 (td, J=7.3, 2.6 Hz, 2H), 1.65 (d, J=6.5 Hz, 3H). ¹³C NMR (201 MHz, (CD₃)₂SO): δ 155.68, 152.28, 149.23, 147.27, 142.10, 141.36, 134.10, 131.72, 128.79, 128.40, 126.93, 105.72, 104.71, 103.60, 82.90, 71.92, 69.63, 29.73, 21.49, 15.26. HRMS m/z (ESI): calcd. for C₂₂H₂₀N₅O₆ [M+H]⁺: 450.1408; found: 450.1409.

### 1-(6-nitrobenzo[d][1,3]dioxol-5-yl)ethyl 6-(but-3-yn-1-yl)-3-(3-(trifluoromethyl)phenyl)-1,2,4,5-tetrazine-1(4H)-carboxylate

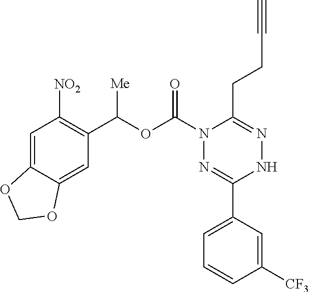 [SMILES: C#CCCC1=NNC(c2cccc(C(F)(F)F)c2)=NN1C(=O)OC(C)c1cc2c(cc1[N+](=O)[O-])OCO2]

¹H NMR (800 MHz, (CD₃)₂SO): δ 10.47 (s, 1H), 8.20 (s, 1H), 8.17 (d, J=8.0 Hz, 1H), 7.97 (d, J=7.8 Hz, 1H), 7.78 (t, J=7.9 Hz, 1H), 7.61 (s, 1H), 7.24 (s, 1H), 6.23 (m, 3H), 2.84 (t, J=7.3 Hz, 2H), 2.80 (s, 1H), 2.40 (t, J=7.6 Hz, 2H), 1.66 (d, J=6.5 Hz, 3H). ¹³C NMR (201 MHz, (CD₃)₂SO): δ 154.40, 152.26, 149.15, 147.29, 142.34, 141.42, 133.86, 130.93, 130.20, 129.71, 129.46, 128.31, 123.42, 105.80, 104.74, 103.61, 82.85, 71.99, 69.89, 29.75, 21.48, 15.28. HRMS m/z (ESI): calcd. for C₂₃H₁₉F₃N₅O₆ [M+H]⁺: 518.1282; found: 518.1278.

### 1-(6-nitrobenzo[d][1,3]dioxol-5-yl)ethyl 6-(but-3-yn-1-yl)-3-(pyridin-4-yl)-1,2,4,5-tetrazine-1(4H)-carboxylate

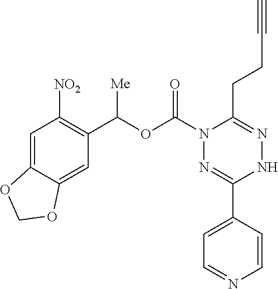 [SMILES: C#CCCC1=NNC(c2ccncc2)=NN1C(=O)OC(C)c1cc2c(cc1[N+](=O)[O-])OCO2]

¹H NMR (800 MHz, (CD₃)₂SO): δ 10.46 (s, 1H), 8.63-8.51 (m, 2H), 7.56 (s, 1H), 7.40 (d, J=5.1 Hz, 2H), 6.23 (d, J=9.5 Hz, 2H), 6.10 (q, J=6.4 Hz, 1H), 2.85 (s, 1H), 2.5-2.50 (m, 4H), 1.44 (brs, 3H). ¹³C NMR (201 MHz, (CD₃)₂SO): δ 160.37, 152.10, 149.83, 147.22, 141.09, 138.58, 120.59, 105.46, 104.56, 103.57, 82.56, 72.16, 69.72, 28.42, 21.50, 14.85. HRMS m/z (ESI): calcd. for C₂₁H₁₉N₆O₆ [M+H]⁺: 451.1361; found: 451.1359.

### 1-(6-nitrobenzo[d][1,3]dioxol-5-yl)ethyl 3,6-diphenyl-1,2,4,5-tetrazine-1(4H)-carboxylate

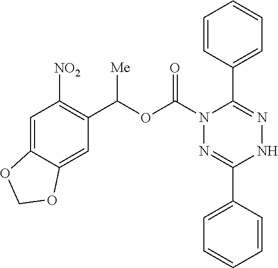 [SMILES: CC(OC(=O)N1N=C(c2ccccc2)NN=C1c1ccccc1)c1cc2c(cc1[N+](=O)[O-])OCO2]

¹H NMR (800 MHz, (CD₃)₂SO): δ 10.88 (s, 1H), 7.92 (d, J=7.7 Hz, 2H), 7.62 (t, J=7.4 Hz, 1H), 7.59-7.30 (m, 8H), 6.48 (brs, 1H), 6.22 (d, J=20.0 Hz, 2H), 6.12 (d, J=6.3 Hz, 1H), 1.42 (brs, 3H). ¹³C NMR (201 MHz, (CD₃)₂SO): δ 158.94, 152.08, 148.72, 147.18, 141.72, 141.06, 132.01, 129.90, 128.88, 128.51, 128.18, 127.28, 126.51, 105.48, 104.55, 103.56, 69.65, 21.60. HRMS m/z (ESI): calcd. for C₂₄H₂₀N₅O₆ [M+H]⁺: 474.1408; found: 474.1406.

### 1-(6-nitrobenzo[d][1,3]dioxol-5-yl)ethyl 3,6-di(pyridin-2-yl)-1,2,4,5-tetrazine-1(4H)-carboxylate

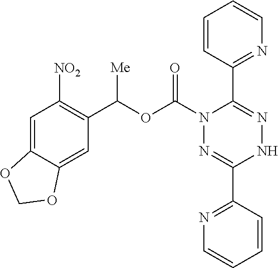 [SMILES: CC(OC(=O)N1N=C(c2ccccn2)NN=C1c1ccccn1)c1cc2c(cc1[N+](=O)[O-])OCO2]

¹H NMR (600 MHz, (CD₃)₂SO): δ 10.63 (s, 1H), 8.71 (dd, J=4.8, 0.8 Hz, 1H), 8.43 (ddd, J=4.8, 1.7, 0.9 Hz, 1H), 8.13-8.09 (m, 1H), 8.03 (td, J=7.7, 1.7 Hz, 1H), 7.90 (td, J=7.7, 1.8 Hz, 1H), 7.76 (d, J=7.8 Hz, 1H), 7.64 (ddd, J=7.5, 4.8, 1.2 Hz, 1H), 7.54 (s, 1H), 7.40 (ddd, J=7.6, 4.8, 1.1 Hz, 1H), 6.57 (s, 1H), 6.23 (dd, J=15.0, 0.9 Hz, 2H), 6.02 (q, J=6.4 Hz, 1H), 1.28 (d, J=6.4 Hz, 3H). ¹³C NMR (151 MHz, (CD₃)₂SO): δ 155.19, 152.01, 149.64, 149.07, 148.82, 148.65, 147.15, 145.89, 141.48, 140.99, 137.91, 137.29, 133.34, 126.72, 124.58, 122.87, 122.06, 105.57, 104.48, 103.56, 69.70, 21.43. HRMS m/z (ESI): calcd. for C₂₂H₁₈N₇O₆[M+H]⁺: 476.1313; found: 476.1311.

### (7-(diethylamino)-2-oxo-2H-chromen-4-yl)methyl 6-(but-3-yn-1-yl)-3-phenyl-1,2,4,5-tetrazine-1(4H)-carboxylate

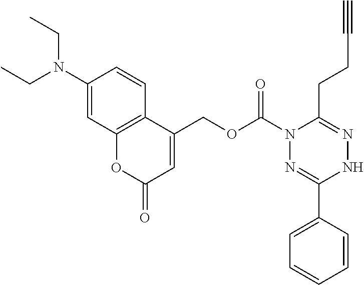 [SMILES: C#CCCC1=NNC(c2ccccc2)=NN1C(=O)OCc1cc(=O)oc2cc(N(CC)CC)ccc12]

¹H NMR (800 MHz, (CD₃)₂SO): δ 10.36 (s, 1H), 7.86 (d, J=7.8 Hz, 2H), 7.59 (t, J=7.6 Hz, 1H), 7.54 (dd, J=9.1, 1.5 Hz, 1H), 7.51 (t, J=7.6 Hz, 2H), 6.70 (dt, J=9.1, 2.1 Hz, 1H), 6.56 (t, J=2.1 Hz, 1H), 6.05 (s, 1H), 5.47 (s, 2H), 3.43 (q, J=7.0 Hz, 4H), 2.90 (t, J=7.2 Hz, 2H), 2.81 (m, 1H), 2.45 (td, J=7.3, 2.6 Hz, 2H), 1.12 (t, J=7.2 Hz, 6H). ¹³C NMR (201 MHz, (CD₃)₂SO): δ 160.62, 155.86, 155.72, 150.72, 150.51, 149.65, 142.05, 131.77, 128.80, 128.34, 126.86, 125.62, 108.75, 105.20, 104.96, 96.87, 82.90, 71.98, 62.91, 44.00, 29.64, 15.32, 12.31. HRMS m/z (ESI): calcd. for C₂₇H₂₈N₅O₄ [M+H]⁺: 486.2136; found: 486.2138.

### (7-(diethylamino)-2-oxo-2H-chromen-4-yl)methyl 3,6-diphenyl-1,2,4,5-tetrazine-1(4H)-carboxylate

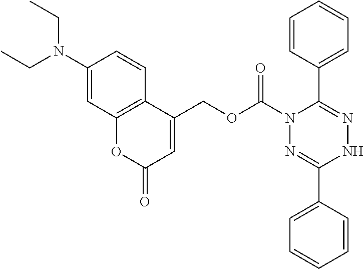 [SMILES: CCN(CC)c1ccc2c(COC(=O)N3N=C(c4ccccc4)NN=C3c3ccccc3)cc(=O)oc2c1]

¹H NMR (800 MHz, (CD₃)₂SO): δ 11.00 (s, 1H), 7.97-7.89 (m, 2H), 7.63 (t, J=7.5 Hz, 1H), 7.59-7.52 (m, 3H), 7.52-7.24 (m, 5H), 6.62 (dd, J=9.1, 2.6 Hz, 1H), 6.52 (d, J=2.5 Hz, 1H), 5.71 (s, 1H), 5.36 (s, 2H), 3.41 (q, J=7.1 Hz, 4H), 1.10 (t, J=7.0 Hz, 6H). ¹³C NMR (201 MHz, (CD₃)₂SO): δ 160.44, 155.75, 150.42, 149.32, 141.47, 132.07, 129.98, 128.88, 128.50, 128.14, 127.27, 126.45, 125.47, 108.67, 105.10, 104.89, 96.80, 62.92, 43.98, 12.30. HRMS m/z (ESI): calcd. for C₂₉H₂₈N₅O₄ [M+H]⁺: 510.2136; found: 510.2131.

### (1,3,5,5,7,9-hexamethyl-5H-4λ⁴,5λ⁴-dipyrrolo[1,2-c:2′,1′-f][1,3,2]diazaborinin-10-yl)methyl 6-(but-3-yn-1-yl)-3-phenyl-1,2,4,5-tetrazine-1(4H)-carboxylate

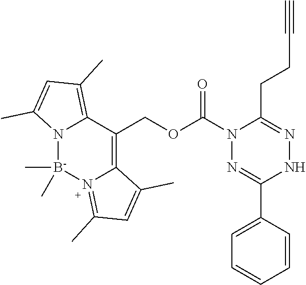

HRMS m/z (ESI): calcd. for C₂₉H₃₄BN₆O₂[M+H]⁺: 509.2836; found: 509.2833.

1. A method of making a tetrazine compound, said method comprising irradiating a photocaged dihydrotetrazine compound with light.

2. The method of claim 1, wherein the photocaged dihydrotetrazine compound has the formula: 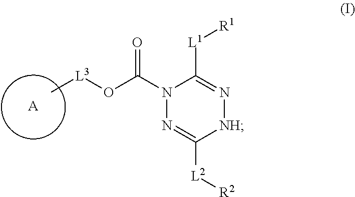
and
the tetrazine compound has the formula: 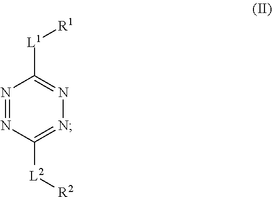
wherein
Ring A is a photolabel moiety;
L¹ is a bond or covalent linker;
L² is a bond or covalent linker;
L³ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X¹ and X² are independently —F, —Cl, —Br, or —I;
n1 and n2 are independently an integer from 0 to 4; and
m1, m2, v1, and v2 are independently 1 or 2.

3. (canceled)

4. (canceled)

5. The method of claim 2, wherein L³ is 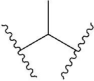 [SMILES: *C(*)C]
or unsubstituted methylene.

6.-8. (canceled)

9. The method of claim 2, wherein the photolabel moiety is

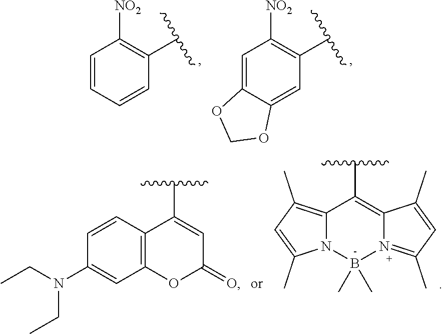

10. The method of claim 2, wherein L¹ is -L¹⁰¹-L¹⁰²-L¹⁰³-L¹⁰⁴-L¹⁰⁵-; and
L¹⁰¹, L¹⁰², L¹⁰³, L¹⁰⁴, and L¹⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

11.-13. (canceled)

14. The method of claim 2, wherein L¹ is

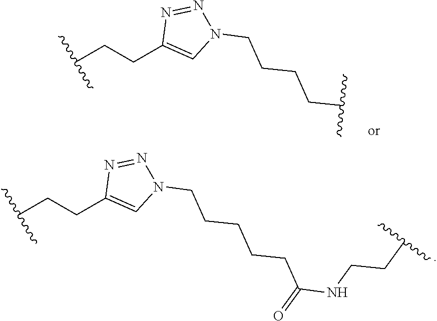 [SMILES: *CCCCn1cc(CC*)nn1 ; *CCNC(=O)CCCCCn1cc(CC*)nn1]

15. The method of claim 2, wherein L² is -L²⁰¹-L²⁰²-L²⁰³-L²⁰⁴-L²⁰⁵-; and
L²⁰¹, L²⁰², L²⁰³, L²⁰⁴, and L²⁰⁵ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene, or a bioconjugate linker.

16.-19. (canceled)

20. The method of claim 2, wherein R¹ is 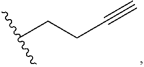 [SMILES: *CCC#C]
unsubstituted phenyl, or unsubstituted pyridyl.

21.-26. (canceled)

27. The method of claim 2, wherein R¹ is

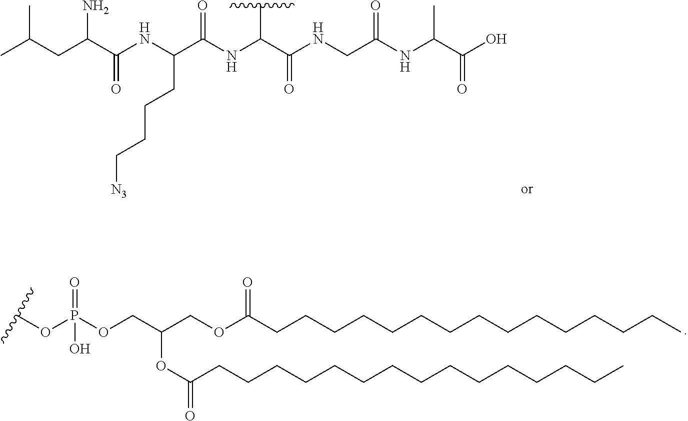 [SMILES: *C(NC(=O)C(CCCCN=[N+]=[N-])NC(=O)C(N)CC(C)C)C(=O)NCC(=O)NC(C)C(=O)O ; *OP(=O)(O)OCC(COC(=O)CCCCCCCCCCCCCCC)OC(=O)CCCCCCCCCCCCCCC]

28.-32. (canceled)

33. The method of claim 2, wherein R² is

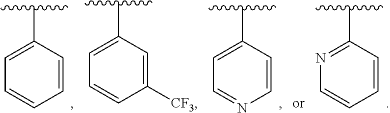

34. The method of claim 1, wherein:
(i) the photocaged dihydrotetrazine compound is 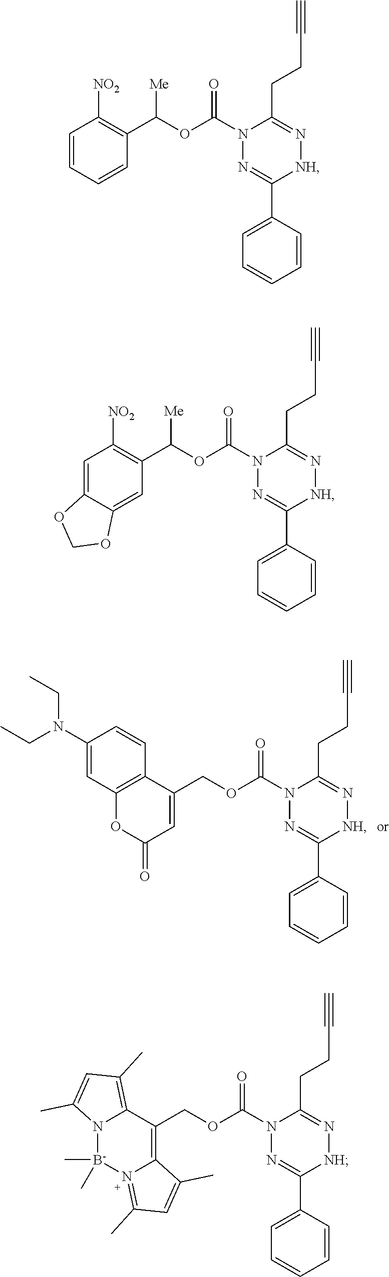
the tetrazine compound 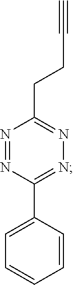 [SMILES: C#CCCc1nnc(-c2ccccc2)nn1]
or
(ii) the photocaged dihydrotetrazine compound is 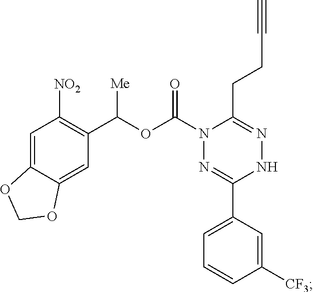 [SMILES: C#CCCC1=NNC(c2cccc(C(F)(F)F)c2)=NN1C(=O)OC(C)c1cc2c(cc1[N+](=O)[O-])OCO2]
and
the tetrazine compound is 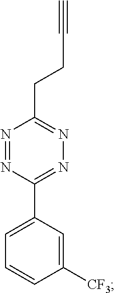 [SMILES: C#CCCc1nnc(-c2cccc(C(F)(F)F)c2)nn1]
or
(iii) the photocaged dihydrotetrazine compound is 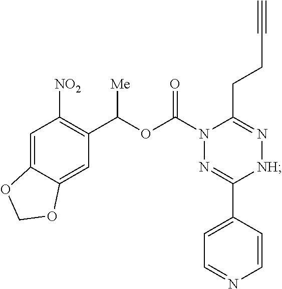 [SMILES: C#CCCC1=NNC(c2ccncc2)=NN1C(=O)OC(C)c1cc2c(cc1[N+](=O)[O-])OCO2]
and
the tetrazine compound is 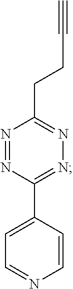 [SMILES: C#CCCc1nnc(-c2ccncc2)nn1]
or
(iv) the photocaged dihydrotetrazine compound is 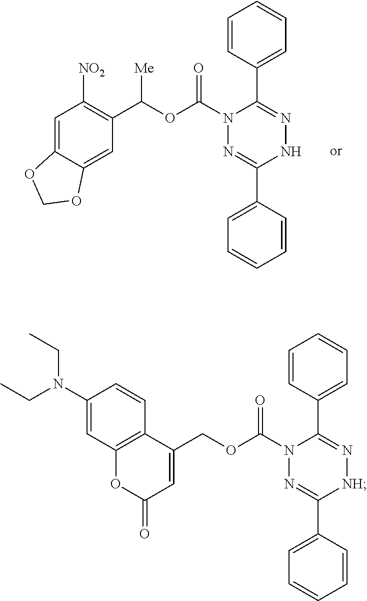 [SMILES: CC(OC(=O)N1N=C(c2ccccc2)NN=C1c1ccccc1)c1cc2c(cc1[N+](=O)[O-])OCO2 ; CCN(CC)c1ccc2c(COC(=O)N3N=C(c4ccccc4)NN=C3c3ccccc3)cc(=O)oc2c1]
and
the tetrazine compound is 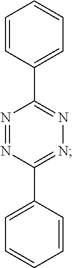 [SMILES: c1ccc(-c2nnc(-c3ccccc3)nn2)cc1]
or
(iv) the photocaged dihydrotetrazine compound is 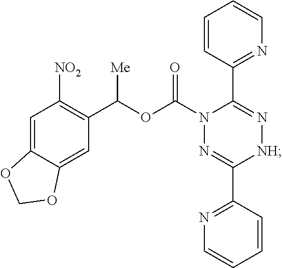 [SMILES: CC(OC(=O)N1N=C(c2ccccn2)NN=C1c1ccccn1)c1cc2c(cc1[N+](=O)[O-])OCO2]
and
the tetrazine compound is 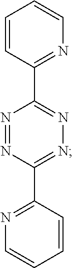 [SMILES: c1ccc(-c2nnc(-c3ccccn3)nn2)nc1]
or
(vi) the photocaged dihydrotetrazine compound is 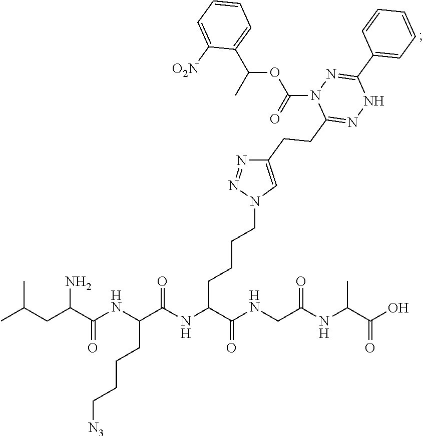 [SMILES: CC(C)CC(N)C(=O)NC(CCCCN=[N+]=[N-])C(=O)NC(CCCCn1cc(CCC2=NNC(c3ccccc3)=NN2C(=O)OC(C)c2ccccc2[N+](=O)[O-])nn1)C(=O)NCC(=O)NC(C)C(=O)O]
and
the tetrazine compound is 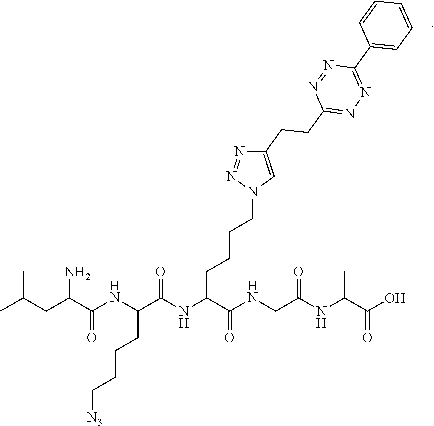 [SMILES: CC(C)CC(N)C(=O)NC(CCCCN=[N+]=[N-])C(=O)NC(CCCCn1cc(CCc2nnc(-c3ccccc3)nn2)nn1)C(=O)NCC(=O)NC(C)C(=O)O]
or
(vii) the photocaged dihydrotetrazine compound is 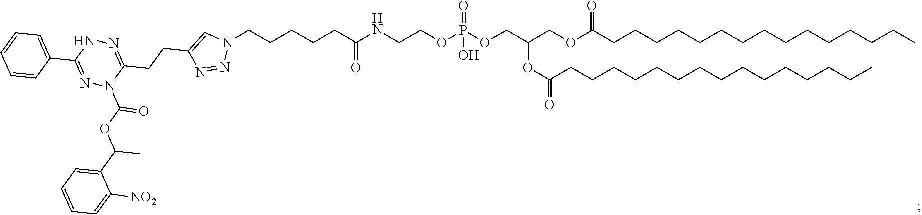 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC]
and the tetrazine compound is 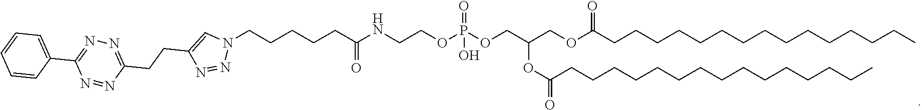 [SMILES: CCCCCCCCCCCCCCCC(=O)OCC(COP(=O)(O)OCCNC(=O)CCCCCn1cc(CCc2nnc(-c3ccccc3)nn2)nn1)OC(=O)CCCCCCCCCCCCCCC]
the photocaged dihydrotetrazine compound is 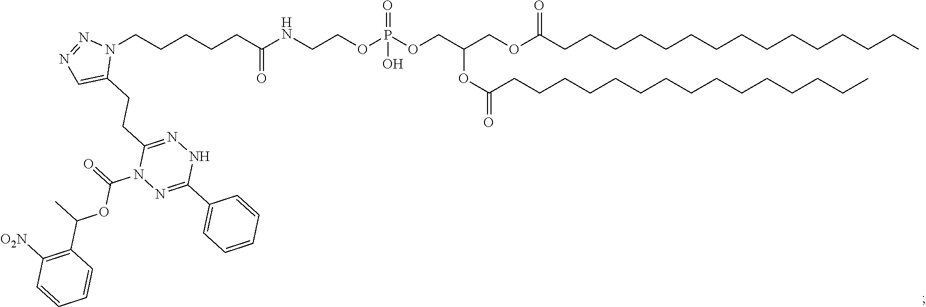 [SMILES: CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC]
and the tetrazine compound is

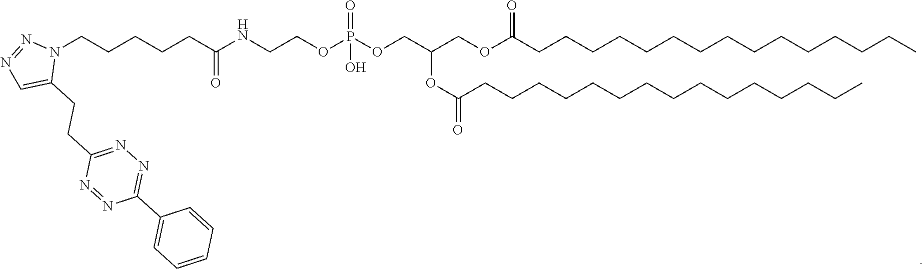 [SMILES: CCCCCCCCCCCCCCCC(=O)OCC(COP(=O)(O)OCCNC(=O)CCCCCn1nncc1CCc1nnc(-c2ccccc2)nn1)OC(=O)CCCCCCCCCCCCCCC]

35.-49. (canceled)

50. The method of claim 1, further comprising reacting the tetrazine compound with a dienophile covalently linked to a monovalent form of a drug or a monovalent form of a probe.

51.-53. (canceled)

54. The method of claim 50, wherein the dienophile covalently linked to a monovalent form of a drug is

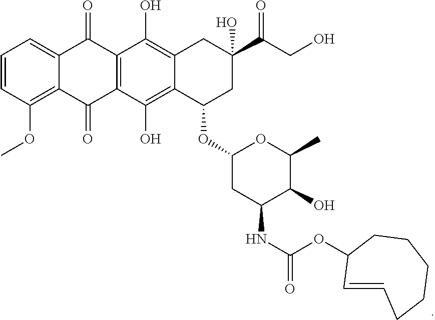 [SMILES: COc1cccc2c1C(=O)c1c(O)c3c(c(O)c1C2=O)C[C@@](O)(C(=O)CO)C[C@@H]3O[C@H]1C[C@H](NC(=O)OC2/C=C/CCCCC2)[C@H](O)[C@H](C)O1]

55. (canceled)

56. The method of claim 50, wherein the dienophile covalently linked to a monovalent form of a probe is

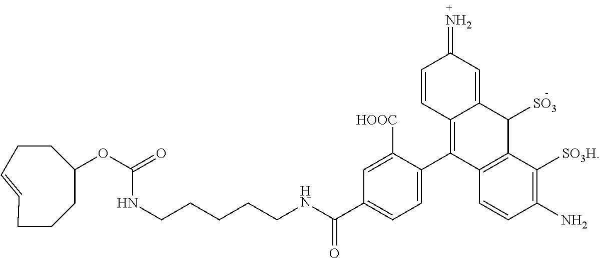

57. (canceled)

58. A compound having the formula: 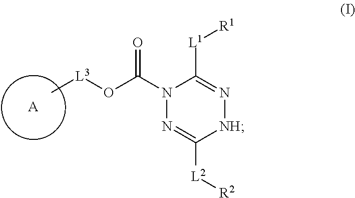
wherein
Ring A is a photolabel moiety;
L¹ is a bond or covalent linker;
L² is a bond or covalent linker;
L³ is a bond, substituted or unsubstituted alkylene, or substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)OR^{1C}, —C(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, —OP(O)(OR^{1C})(OR^{1D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R² is hydrogen, halogen, —CX²₃, —CHX²₂, —CH₂X², —OCX²₃, —OCH₂X², —OCHX²₂, —CN, —SOₙ₂R^{2D}, SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)OR^{2C}, —C(O)NR^{2A}R^{2B}, —OR^{2D}, —SR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, —OP(O)(OR^{2C})(OR^{2D}), substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, or a biomolecular moiety;
R^{1A}, R^{1B}, R^{1C}, R^{1D}, R^{2A}, R^{2B}, R^{2C}, and R^{2D} are independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl;
X¹ and X² are independently —F, —Cl, —Br, or —I;
n1 and n2 are independently an integer from 0 to 4; and
m1, m2, v1, and v2 are independently 1 or 2.

59.-89. (canceled)

90. The compound of claim 58, having the formula:

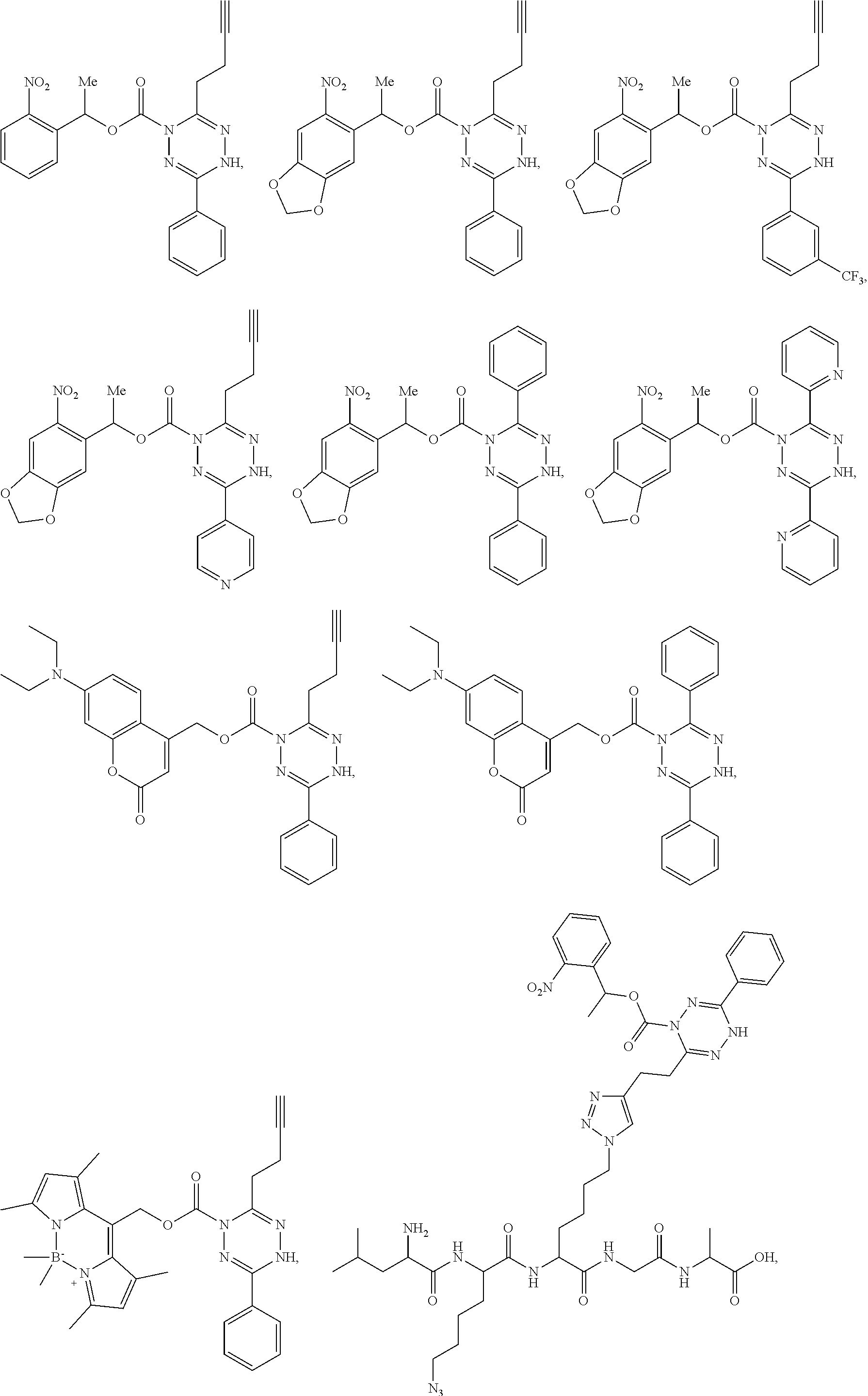

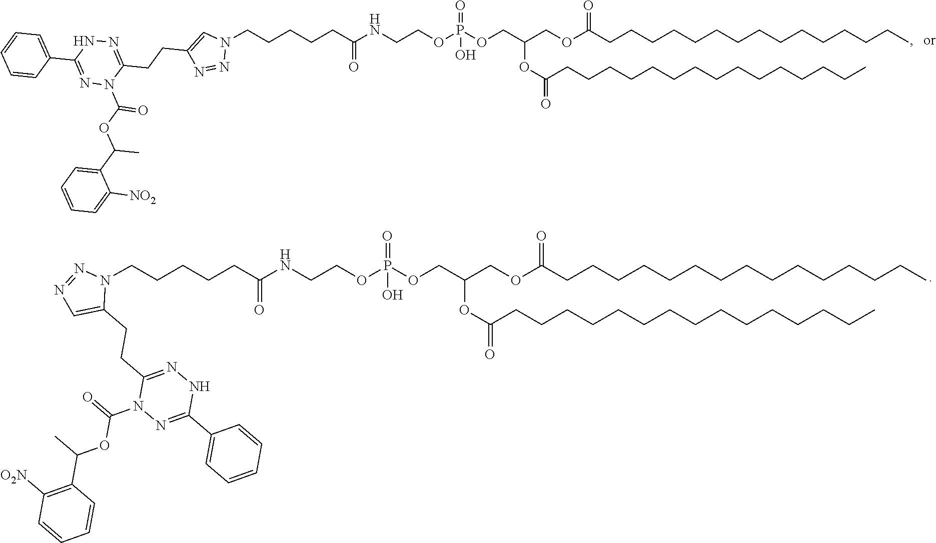 [SMILES: CCCCCCCCCCCCCCCC(=O)OCC(COP(=O)(O)OCCNC(=O)CCCCCn1cc(CCC2=NNC(c3ccccc3)=NN2C(=O)OC(C)c2ccccc2[N+](=O)[O-])nn1)OC(=O)CCCCCCCCCCCCCCC ; CCCCCCCCCCCCCCCC(=O)OCC(COP(=O)(O)OCCNC(=O)CCCCCn1nncc1CCC1=NNC(c2ccccc2)=NN1C(=O)OC(C)c1ccccc1[N+](=O)[O-])OC(=O)CCCCCCCCCCCCCCC]

91.-93. (canceled)

94. A pharmaceutical composition comprising a compound of claim 58, or a pharmaceutically acceptable salt thereof, and a pharmaceutically acceptable excipient.

95. A method of treating a cancer in a subject in need thereof, said method comprising:
(i) administering to the subject in need thereof a photocaged dihydrotetrazine compound or a compound of claim 58, or a pharmaceutically acceptable salt thereof,
(ii) irradiating the photocaged dihydrotetrazine compound or the compound with light to form a tetrazine compound; and
(iii) reacting the tetrazine compound with a dienophile covalently linked to a monovalent form of an anti-cancer agent, thereby releasing the anti-cancer agent.

96. The method of claim 95, wherein the cancer is hepatocellular carcinoma, cervical carcinoma, kidney cancer, or skin cancer.

97.-106. (canceled)

107. A method of delivering a drug or a probe in a subject in need thereof, said method comprising:
(i) administering to the subject in need thereof a photocaged dihydrotetrazine compound or a compound of claim 58, or a pharmaceutically acceptable salt thereof,
(ii) irradiating the photocaged dihydrotetrazine compound or the compound with light to form a tetrazine compound; and
(iii) reacting the tetrazine compound with a dienophile covalently linked to a monovalent form of the drug or a monovalent form of the probe, thereby releasing the drug or the probe.

108.-118. (canceled)
